(19) United States
(12) Patent Application Publication
(10) Pub. No.: US 2023/0220313 A1
(43) Pub. Date: Jul. 13, 2023

(54) COMPOSITION AND METHOD FOR INACTIVATING A VIRUS

(71) Applicants: Andrew Simon GOLDSBOROUGH, Talence (FR); Malcolm Robert BATES, Cambridge (GB); RNASSIST LIMITED, Cambridge (GB)

(72) Inventors: Andrew Simon Goldsborough, Cambridge (GB); Malcolm Robert Bates, Cambridge (GB)

(21) Appl. No.: 17/918,017

(22) PCT Filed: Apr. 12, 2021
(86) PCT No.: PCT/EP2021/059465

§ 371 Date: Oct. 10, 2022

(30) Foreign Application Priority Data
Apr. 10, 2020 (GB) 2005356.7
Dec. 15, 2020 (GB) 2019821.4

(51) Int. Cl. C11D 7/32; C11D 3/48; C11D 7/34; C11D 7/26

(52) U.S. Cl. CPC C11D 7/3209; C11D 3/48; C11D 7/34; C11D 7/263

(57) ABSTRACT

Provided is the use of a composition to inactive a virus. The composition comprises a quaternary ammonium compound and a halogenated organic compound. Also provided are a composition and kit for inactivating a virus, and a method for detecting an analyte in a sample.

### BACKGROUND

Working with SARS-CoV-2 is restricted to high-containment laboratories, with material only permitted to be used at a lower containment level if it has first been inactivated. Infection with SARS-CoV-2 causes the fatal respiratory disease COVID-19 (Huang C., et al (2020) Lancet 395:497-506). Worldwide, tens of millions of clinical samples have been collected for diagnostic testing. SARS-CoV-2 is a hazard group 3 pathogen so must be handled in a high-containment CL3 (BSL-3) laboratory. The Centers for Disease Control permit diagnostic testing in a CL3 (BSL-3) lab but infectious material must be handled in a microbiological safety cabinet unless inactivated first.

It is known that SARS-CoV-1 and Middle East respiratory syndrome coronavirus (MERS-CoV) can be inactivated with heat, UV, chemicals, gamma-irradiation and detergents (Rabeneau H F., (2005) Biologicals 33:95-99; Kumar M., (2015) J. Virol Methods. 223:13-18). However, it is also known that SARS-CoV-2 is not completely inactivated by heat, powerful chaotropes such as Guanidine thiocyanate and Guanidine HCl, detergents such as SDS, Triton X-100, as well as many commercial viral lysis buffers such as BufferAVL (Qiagen, Germany) as described by Welch, S R., et al (2020) J. Clin. Microbiology, Vol 58:e017113-20.

Virus sample transport tubes should be able to inactivate pathogens in clinical specimens before sample transport and also, preferably, preserve the integrity of analytes including nucleic acids, especially RNA as well as proteins for testing. The BS EN 14476 standard requires a >4-log 10 titer reduction for virucidal suspension tests (British Standards Institution (2019) Chemical disinfectants and antiseptics, London UK).

During the current SARS-CoV-2 pandemic there is an unprecedented need to rapidly, safely and test for the presence of the virus at a population wide level. All diagnostic tests whether they detect nucleic acid, protein or another analyte are reliant on a sample, whether obtained for example from cells, tissues, swabs, or a naturally occurring liquid such as blood, sputum, or saliva. Clinical specimens from an infected individual are a biosafety risk and can potentially spread the disease to close contacts, those handling the samples such as delivery personnel, as well as those handling the tube on arrival at the testing laboratory, particularly once the container holding the sample is opened to remove and test the analyte sample.

A significant number of SARS-CoV-2 sample collection tubes arriving in testing laboratories leak during transport and are therefore a significant biohazard. Inactivating the SARS-CoV-2 virus, as well as other potential pathogens in the sample such as Mycobacterium tuberculosis, Candida albicans, Hepatitis and HIV significantly reduces biohazards therefore improving operator safety. It is also essential to preserve in the sample all analytes such as RNA, DNA and/or protein required for the diagnostic test during transport and handling.

Currently, FDA guidance suggests the use of aqueous Viral Transport Medium (VTM) that do not inactivate or preserve the analyte in the sample but simply serve as a buffer to transport the sample prior to testing. Inactivating Transport Mediums (ITMs) can inactivate viruses but do not necessarily preserve the analyte molecules. Molecular Transport Mediums (MTMs) may preserve the analyte but do not necessarily inactivate viruses. Universal Transport Mediums (UTMs) can be used to transport viruses, bacteria and other pathogens but without inactivating or killing the microbe (Welch, SR., et al (2020) J. Clin. Microbiology, Vol 58:e017113-20). Many TMs are used simply to transport live microbes such as viruses, bacteria, fungi and parasites including pathogens to a testing laboratory so they can be cultured for identification.

There remains a need for compositions which can inactivate a pathogen while at the same time stabilising biomolecules present in the pathogen such as RNA or DNA to allow for the subsequent detection of the pathogen.

Detection of a pathogen may comprise molecular diagnostic testing. Molecular diagnostic nucleic acid testing commonly includes the following steps:
(i) sampling from a patient;
(ii) transport and storage;
(iii) RNA and/or DNA extraction; and
(iv) analysis of the RNA and/or DNA.

During the current SARS-CoV-2 virus pandemic there have been widespread world-wide shortages of reagents and consumables necessary for all four of these steps, leading to greatly reduced population testing, increased costs, longer waiting times for test results, and substitution of inferior or inappropriate reagents for recommended products.

Once the crude clinical sample has reached the testing centre, the most labour intensive and hands-on step is RNA and/or DNA purification. Although automation is possible, this is generally only used in very high throughput laboratories and remains expensive and highly dependent on reagent and consumable availability. Turnaround time from sampling to result is at a premium when the patient is in a medical setting such as a point-of-care and needs to be triaged as quickly as possible to optimise hospital resources as well as potentially needing to be quarantined or receive emergency therapeutic intervention or respiratory aid.

Various commercialised tests exist for using whole cells directly in a reverse transcription reaction, the process of converting or copying an RNA sequence into its corresponding DNA (cDNA) copy. These tests include Cells-to-cDNA II™ (ThermoFisher), SMART-seq-2 (Simone Picelli et al., (2019) Methods Mol Biol. 2019; 1979:25-44), CEL-seq, MARS-seq, and those commercialised by Takara Biosciences as SMARTer-seq™, PrimeDirect™, other single cell RNA-seq and single cell DNA-seq methods including drop-seq (Zillionis et al., (2017) and commercialised kits from 10× Genomics, Mission Bio, Celsee, Becton Dickerson and Bio-Rad Laboratories. All of these methods require cell lysis, and particularly in the case of DNA cell and nuclear lysis in order to release the desired analyte RNA and/or DNA which can then be copied by a polymerase reagent in vitro, frequently in an emulsion droplet nanowell or multiwell plate, and ultimately detected by a variety of means such as sequencing or using a fluorescent dye.

Using RNA or DNA directly from a virus, a sample containing a virus or viruses, a cell, a sample containing a cell or cells, a virus and a cell, and/or a sample containing a virus or viruses and a cell or cells, without prior nucleic acid purification requires the researcher to overcome the considerable potential negative effects of using non-purified samples such as: (i) enzyme inhibitors, (ii) lower amounts of detectable nucleic acid, (iii) nucleic acid structure, (iv) non-desired proteins and RNA bound to the desired nucleic acid, which can interfere with detection of the desired nucleic acid; (v) enzyme contamination particularly by nucleases such as ribonucleases, (vi) instability of the sample during storage and transport, and (vii) biosafety issues when handling infectious and pathogenic samples that have not been purified.

Despite these drawbacks, direct analysis of nucleic acids in non-purified samples are employed because they offer simplicity, speed, reduced cost and avoidance of reagents that may be in limited supply as in the current COVID-19 pandemic. It will therefore be evident that there is a trade-off between lower cost, greater speed and reagent availability on the one hand, with analytical specificity and sensitivity on the other. However for many applications, particularly when there is the need to generate hundreds of thousands of viral test results per day in resource constrained settings or during pandemics, such trade-offs can be managed and become acceptable in order to overcome reagent shortages and other road-blocks on the way to a diagnostic result.

It would be desirable to provide a method which allows detection of an analyte in a non-purified sample which addresses limitations of the existing methods.

### SUMMARY

In one aspect, there is provided the use of a composition to inactive a virus, which composition comprises:
a quaternary ammonium compound; and
a halogenated organic compound;

wherein the quaternary ammonium compound is a compound of Formula 1 or a salt thereof:

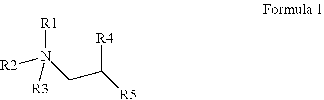

wherein:
R1, R2 and R3 are each independently selected from a methyl group and ethyl group;
R4 is H or OH;
R5 is selected from H, a halide, a carbonyl oxygen, a methyl group, and

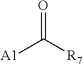

wherein:
   A1 is selected from CH₂, O and S;
   R7 is selected from OH, an unsubstituted C1 to C3 alkyl group, an alkyl carboxylic acid group having 1 to 3 carbon atoms, and a substituted C1 to C3 alkyl group bearing one or more substituents selected from hydroxyl and halide groups;
   with the proviso that, when R5 is H or a methyl group, R4 is OH;

and wherein the halogenated organic compound is a compound of Formula 2:

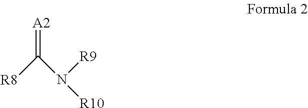

wherein:
A2 is O or S;
R8 is a substituted C1 to C12 alkyl group bearing one or more substituents selected from fluoro, chloro, bromo, and iodo; and
   i) R9 and R10 are each independently selected from H; an unsubstituted C1 to C12 alkyl group; an unsubstituted alkyl ester group having 2 to 12 carbon atoms; an alkyl ester group having 2 to 12 carbon atoms and bearing one or more substituents selected from fluoro, chloro, bromo, iodo, and hydroxyl; and a substituted C1 to C12 alkyl group bearing one or more substituents selected from fluoro, chloro, bromo, iodo, and hydroxyl; or
   ii) R9 and R10 together form an imidazole group.

The composition may be a deep eutectic solvent, or a “classical” solution comprising the quaternary ammonium compound and the halogenated compound dissolved in a solvent. The solvent may be an aqueous solvent or a non-aqueous solvent. In implementations where it is desired to analyse the virus, the solvent is preferably a non-aqueous solvent. It has been found that biomolecules such as RNA are better stabilised when the solvent is non-aqueous.

Solid mixtures of the halogenated organic compound and quaternary ammonium compound are also contemplated. Also contemplated are semi-solid mixtures, such as gels.

The non-aqueous solvent may comprise glycerol, ethylene glycol, diethylene glycol, triethylene glycol, and/or a polyethylene glycol. For example, the solvent may be a polyethylene glycol having a molecular weight in the range 150 to 600, optionally 200 to 600.

As used herein, a solution is considered “non-aqueous” even if it comprises trace amounts of water, e.g. up to 10% water by volume based on the volume of the solution, preferably no more than 1% water. A hygroscopic solvent which takes up a small amount of water from the air is still regarded as “non-aqueous” for the present purposes.

A further example of a class of useful non-aqueous solvents is the polyethylene glycol alkyl ethers. For example, the solvent may be an alkyl ether of tetraethylene glycol. Alkyl ethers of polyethylene glycols have lower viscosity than the corresponding polyethylene glycol. Lower viscosity may allow easier handling of the composition.

The concentration of the quaternary ammonium compound in the composition may be in the range 1 mM to 8 M, 20 mM to 1.2 M, 80 mM to 1 M, preferably 0.1 to 400 mM.

The concentration of the halogenated organic compound in the solvent may be in the range 0.1 mM to 8 M, 1 mM to 8 M, 20 mM 1.2 M, 80 mM to 1 M, or 80 mM to 850 mM. Compositions in which the concentration is less than or equal to 850 mM may be particularly safe to handle, while still effectively inactivating pathogens.

The composition may further comprise a solvent having a structure of Formula 3:

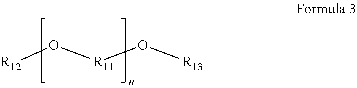

wherein:
R11 is a methylene group or a C2 to C6 linear or branched alkylene group;
R12 and R13 are each independently selected from H, a C1 to C6 alkyl group, an acrylate group, a methacrylate group, and an oxolan-2-ylmethylene group; and
wherein n is in the range 1 to 14,

with the proviso that when n is 1, R11 is not methylene or at least one of R12 and R13 is not H.

The solvent may be a polymer, i.e., n may be 2 or more. In such implementations, n may represent an average chain length. For example, one preferred solvent is PEG200. PEG 200 is a mixture of polyethylene glycols having chain lengths in the range 1 to 10, but an average molecular weight of about 200.

The solvent may be a cosolvent mixture. The cosolvent mixture may comprise two or more different solvents according to Formula 3. One particularly useful mixture comprises tetraethylene glycol and tetraethylene glycol monomethyl ether. The tetraethylene glycol and tetraethylene glycol monomethyl ether may be present at a ratio in the range 1:2 to 2:1, optionally 1:1, by volume based on the volume of the mixture.

As used herein, the term “oxolan-2-ylmethylene group” refers to the following group:

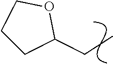

When the solvent comprises an oxolan-2-ylmethylene group, it may be described as an ether of tetrahydrofurfuryl alcohol. For example, the solvent may be tetrahydrofurfuryl alcohol polyethyleneglycol ether.

R12 and R13 may each independently selected from H, a methyl group, an ethyl group, and a methacrylate group.

For example, one of R12 and R13 may be a methyl group, and the other of R12 and 13 may be H. Alternatively, R12 may be a methyl group and R13 may be a methyl group. In accordance with a still further possibility, R12 and R13 may each be methacrylate groups.

R11 may be a C2 linear alkylene group. In other words, the solvent may be ethylene glycol, polyethylene glycol, or a derivative thereof.

R11 may be a C3 branched alkylene group. In other words, the solvent may be a propylene glycol or polypropylene glycol, or a derivative thereof.

N may be in the range 3 to 4. The compound may be a triethylene glycol, a tripropylene glycol, or a derivative thereof.

By “derivative” is meant that R12 and R13 are selected from the above-mentioned groups, with the proviso that at least one of R12 and R13 is not H. A preferred example of a derivative is a monomethyl ether, in which one of R12 and R13 is H and the other of R12 and R13 is methyl.

The solvent has a molecular weight in the range 50 to 600 Da. Polyethylene glycols having molecular weights in this range are preferred.

The solvent may be selected from: tripropylene glycol, tripropylene glycol monomethyl ether, tripropylene glycol dimethyl ether, tetrapropylene glycol, tetrapropylene glycol monomethyl ether, tetrapropylene glycol dimethyl ether, tetraethylene glycol, and tetraethylene glycol monomethyl ether.

A further example of a useful solvent is a polypropylene glycol-polyethylene glycol co-polymer.

Still further examples of solvents include water, C1 to C6 alkanols, and mixtures thereof. For example, the solvent may be ethanol, or a mixture comprising water and ethanol. The ratio of water to ethanol may be in the range 2:1 to 1:2 by volume.

The amount of solvent is not particularly limited. The composition may comprise the solvent in an amount of 10 to 99%, optionally 50 to 98%, further optionally 80 to 95% by volume based on the volume of the composition.

The virus may be a coronavirus. The coronavirus may be severe acute respiratory syndrome coronavirus 2, SARS-CoV-2.

The virus may be present in a biological sample. The nature of the biological sample is not particularly limited. For example, the biological sample may comprise mucus, sputum, saliva, and/or breath condensate. These biological samples are particularly relevant in the context of testing for respiratory diseases, such as COVID-19. Breath condensate may be preferred, because breath condensate contains substantially fewer background components originating from the host organism (for example, enzymes) compared to other biological samples, such as saliva.

The biological sample may be present on a swab. A swab comprises a pad of absorbent material, such as nylon, viscose, rayon, cotton, or a combination thereof. The swab may be a nasal swab, nasopharyngeal swab or oropharyngeal swab, further comprising an elongate shaft configured to hold the pad of absorbent material.

The ratio of the amount of composition to the biological sample is not particularly limited. In the case of saliva samples, it has been found that adding a composition in an amount of 2.5 to 3.5% by volume based on the volume of the resultant mixture may significantly reduce the viscosity of the sample. This effect has been observed in particular for the deep eutectic solvent N,N,N-trimethylglycine:trifluoroacetamide. Reducing the viscosity of a sample may make the sample easier to handle, e.g. by pipetting.

The use may further comprise, after inactivating the virus, analysing the biological sample to detect or identify the virus and its genetic variants.

The analysis may include an amplification reaction, such as polymerase chain reaction (“PCR”), for example a reverse transcription polymerase chain reaction (“RT-PCR”); or a loop-mediated isothermal amplification (“LAMP”) such as a reverse transcription loop-mediated isothermal amplification (“RT-LAMP”). Alternatively or additionally, the analysis may comprise an antigen or antibody test, for example a lateral flow test. The composition may be effective for inactivating the virus, while still allowing the virus to be identified.

The use may further comprise, after inactivating the pathogen and before analysing the biological sample, storing the biological sample for a period of time in the range 2 hours to 72 hours. The composition may inhibit degradation of biomolecules such as RNA, DNA and/or protein in the sample, thereby allowing for storage or transport of the sample before the analysis.

Optionally, no purification is performed between the inactivation and the analysis. Compositions provided herein may not interfere with analytical processes, and separating the virus from the composition may not be needed. In particular, purification may be omitted when the concentration of the halogenated organic compound is in the range 0.1 to 850 mM.

Alternatively, the use may comprise, after inactivating the pathogen, separating the inactivated pathogen from the composition. One useful technique for separating the inactivated pathogen from the composition is size exclusion chromatography.

The use may be to disinfect a surface. Disinfecting the surface may comprise contacting the surface with the composition, for example, wiping the surface with the solution. The nature of the surface to be disinfected is not particularly limited. Examples include worksurfaces, medical equipment, personal protective equipment (“PPE”), and skin. Since the compositions may be effective for inactivating pathogens such as viruses, the compounds are useful as disinfectants or germicides.

The use may be to disinfect a medical device, for example a diagnostic device. The diagnostic device may be a lateral flow device, e.g. a Lateral Flow Rapid Test. For example, a lateral flow test may be performed using a non-inactivated sample.

After performing the lateral flow test, the device may be disinfected, for example by introducing the composition into a loading port of the device. This may allow the test to be disposed of as ordinary waste, rather than as infectious waste. This may avoid the need for infectious waste bins in public settings such as at airports.

Alternatively or additionally, the composition may be used to preserve the result on the device for future reference. This may address the problem that lateral rapid flow tests typically remain readable only for about a day: gold bead lines commonly used in rapid test devices otherwise fade quickly, and nitrocellulose membranes used in rapid lateral flow test devices quickly discolour and turn yellow.

The ability of the compositions described herein to prevent the degradation of biomolecules may allow for subsequent extraction of the virus from the used test. For example, the virus may be recuperated from the device by extracting the RNA from the membrane. A portion of the membrane corresponding to a positive result line could be excised from the device. The bound virus may be extracted from the membrane, e.g. lysed form the membrane. The virus could then be analysed by an appropriate technique such as RT-PCR. The lateral flow test device may serve to partially purify the virus. This process may allow for a retrospective analysis of positive lateral flow tests.

Another possibility would be to inactive the virus before performing a lateral flow test.

The molar ratio of the quaternary ammonium compound to the halogenated organic compound is not particularly limited. The molar ratio may be a “stoichiometric” ratio, that is, a ratio which would form a deep eutectic solvent. Stoichiometric molar ratios may be in the range 1:3 to 2:1, optionally 1:1.5 to 1:2.5, further optionally about 1:2.

Alternatively, the molar ratio may be “non-stoichiometric”, in other words, may be a ratio which would not necessarily form a deep eutectic solvent. In such implementations, the composition typically further comprises a solvent. The use of a solid mixture, for example applied to a surface by rubbing, is also contemplated.

The quaternary ammonium compound may be in the form of a salt, further comprising a counterion. The counterion may be selected from: nitrate, tetrafluoroborate, hydroxide, bitartrate, citrate, p-toluenesulfonate, bicarbonate, chloride, bromide, fluoride and iodide. In particular, the counterion may be chloride.

The counterion may be omitted, particularly in implementations where the quaternary ammonium compound comprises a carboxylic acid group. When the quaternary ammonium compound includes a carboxylic acid group, the compound may be in the form of a zwitterion. Particularly preferably, the compound of Formula 1 is zwitterionic.

R1, R2 and R3 may each be independently selected from methyl groups and ethyl groups. Examples of preferred compounds of Formula 1 include those in which R1, R2 and R3 are methyl groups.

The compound of Formula 1 preferably includes a carboxylic acid group. For example, R4 may be OH and R5 may be a carbonyl oxygen.

Further compounds comprising carboxylic acid groups include those where R5 is:

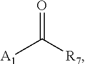

A1 is CH₂, and R7 is OH.

A particularly preferred compound of Formula 1 is N,N,N-trimethylglycine (also referred to herein as “trimethylglycine” or “betaine”). In trimethylglycine, R1, R2 and R3 are methyl groups, R4 is OH and R5 is a carbonyl oxygen.

Alternatively, the compound of Formula 1 may comprise choline or a halocholine. In choline, R4 is OH and R5 is H. In a halocholine, R4 is H and R5 is a halide. The halide may be selected from chloride, bromide and iodide, preferably chloride or bromide.

In examples where R5 is:

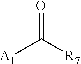

A1 may be O or S, and A is preferably O.

R7 may in particular be selected from OH and an unsubstituted C1 to C3 alkyl group.

The compound of Formula 1 may in particular be selected from: Choline nitrate, Choline tetrafluoroborate, Choline hydroxide, Choline bitartrate, Choline dihydrogen citrate, Choline p-toluenesulfonate, Choline bicarbonate, Choline chloride, Choline bromide, Choline iodide, Choline fluoride, Chlorocholine chloride, Bromocholine bromide, lodocholine iodide, Acetylcholine hydroxide, Acetylcholine bitartrate, Acetylcholine dihydrogen citrate, Acetylcholine p-toluenesulfonate, Acetylcholine bicarbonate, Acetylcholine chloride, Acetylcholine bromide, Acetylcholine iodide, Acetylcholine fluoride, Chloroacetylcholine chloride, Bromoacetylcholine bromide, lodoacetylcholine iodide, Butyrylcholine hydroxide, Butyrylcholine bitartrate, Butyrylcholine dihydrogen citrate, Butyrylcholine p-toluenesulfonate, Butyrylcholine bicarbonate, Butyrylcholine chloride, Butyrylcholine bromide, Butyrylcholine iodide, Butyrylcholine fluoride, ChloroButyrylcholine chloride, BromoButyrylcholine bromide, IodoButyrylcholine iodide, Acetylthiocholine chloride, L-Carnitine, D-Carnitine, Betaine, Betaine HCl, Beta-methylcholine chloride, and Choline citrate.

In particular, the compound of Formula 1 may be Betaine. When betaine is in the form of a salt, the salt may be a hydrochloride salt.

Examples of preferred compounds of Formula 2 include those in which the substituents present in R8 are selected from fluoro and chloro. In examples where R9 and/or R10 comprises a substituted alkyl group, the substituents may in particular be selected from fluoro and chloro.

R8 may be a substituted C1 to C4 alkyl group bearing one or more substituents selected from fluoro, chloro, bromo, and iodo.

R9 and R10 may each independently selected from H, an unsubstituted C1 to C4 alkyl group; and a substituted C1 to C4 alkyl group bearing one or more substituents selected from fluoro, chloro, bromo, iodo, and hydroxyl.

A2 in Formula 2 may in particular represent O.

The compound of Formula 2 may include at least one fluoro substituent, optionally at least two fluoro substituents, further optionally at least three fluoro substituents. The fluoro substituents may be present in any of R8, R9, and R10. For example, at least one of the fluoro substituents may be present in R8.

The compound may have a total of two or three fluoro substituents, preferably three fluoro substituents. The fluoro substituents may be present in R8.

When the compound includes three or more fluoro substituents, one of R8, R9 and R10 may comprise a trifluoromethyl group. R8 may include a trifluoromethyl group. R8 may be a trifluoromethyl group.

R8 may be a substituted C1 alkyl group.

R9 and R10 may be independently selected from H, a substituted C1 alkyl group, or an unsubstituted C1 alkyl group, with H and an unsubstituted C1 alkyl group being preferred. For example, R9 may be H, and R10 may be H or methyl. More preferably, R9 and R10 are both H.

In particular, the halogenated organic compound may be selected from: fluoroacetamide, difluoroacetamide, trifluoroacetamide, trifluorothioacetamide, chloroacetamide, dichloroacetamide, trichloroacetamide, chlorofluoroacetamide, chlorodifluoroacetamide, dichlorofluoroacetamide, N-methyl-fluoroacetamide, N-methyl-difluoroacetamide, N-methyl-trifluoroacetamide, N-methyl-chloroacetamide, N-methyl-dichloroacetamide, N-methyl-trichloroacetamide, N-methyl-chlorofluoroacetamide, N-methyl-chlorodifluoroacetamide, and N-methyl-dichlorofluoroacetamide.

R8 may be a substituted C1 alkyl group, bearing two or three substituents, wherein the substituents are selected from fluoro and chloro. Preferably, R8 may be a substituted C1 alkyl group, bearing two or three substituents, wherein the substituents are fluoro groups.

R9 and R10 may each be independently selected from H, methyl, and a substituted methyl group bearing one or more substituents selected from fluoro and chloro groups. Preferably, R9 may be H, and R10 may be methyl. More preferably, R9 and R10 may each be H.

The halogenated organic compound may be selected from: difluoroacetamide, difluorothioacetamide, trifluoroacetamide, and trifluorothioacetamide. More particularly, the halogenated organic compound may be trifluoroacetamide or trifluorothioacetamide.

A particularly preferred composition comprises betaine (i.e., N,N,N-trimethylglycine) as the quaternary ammonium compound, and trifluoroacetamide as the halogenated organic compound.

There is also provided a method of inactivating a virus, which method comprises contacting the pathogen with a composition comprising a compound of Formula 1 and a compound of Formula 2, whereby the composition inactivates the virus. The method may further comprise analysing the inactivated virus, to identify the virus. Features discussed above with reference to the use aspect are equally applicable to the method aspect.

Another aspect provides a composition comprising an inactivated pathogen obtainable by contacting a pathogen with a composition comprising a quaternary ammonium compound of Formula 1 and a halogenated organic compound of Formula 2. The composition may further comprise the quaternary ammonium compound and/or the halogenated compound.

The composition may be for use in medicine. The composition may be for use in preventing a disease or disorder caused by the pathogen. In particular, the pathogen may be a virus. The virus may be a coronavirus, for example, severe acute respiratory syndrome coronavirus 2 (“SARS-CoV-2”). In such implementations, the disease may be COVID-19.

The composition may comprise a pharmaceutically-acceptable non-aqueous solvent, and optionally one or pharmaceutically-acceptable excipients, such as a preservative. The composition may be formulated for administration by injection.

It is to be appreciated that the compounds and compositions discussed with reference to the use aspect are equally applicable to the composition aspect. In particular, the composition may comprise a solvent of Formula 3, as discussed above with reference to the use aspect.

Another related aspect provides a method of manufacturing a vaccine, which method comprises: contacting a pathogen with a composition to form an inactivated pathogen; and separating the inactivated pathogen from the composition, wherein the composition comprises: a quaternary ammonium compound of Formula 1, a halogenated compound of Formula 2, and optionally a solvent. The method may further comprise, after the separating, combining the inactivated pathogen with a solvent, and optionally one or more pharmaceutically-acceptable excipient. The contacting may comprise a use as described with reference to the first aspect.

Also provided is a method of manufacturing a vaccine for preventing a viral disease, which method comprises: preparing an inactivated virus by the use of a composition comprising a quaternary ammonium compound and a halogenated organic compound as defined as described with reference to the use aspect; and formulating the inactivated virus in a pharmaceutically-acceptable carrier.

The quaternary ammonium compound may be N,N,N-trimethylglycine, and the halogenated organic compound may be trifluoroacetamide. The composition may further comprise ethanol in an amount in the range 1% to 10% by volume based on the total volume of the composition. The virus may be SARS-CoV-2.

It has been found by high performance microscopy that a composition comprising betaine:trifluoroacetamide at a 1:2 mol:mol ratio and diluted with 5% ethanol by volume maintained the viral particle integrity of a sample of SARS-CoV-2. It has also been found that such a composition provides effective viral inactivation. Accordingly, the use as defined herein may be applicable to the purification of viral particles and the manufacture of inactivated viral vaccines.

More generally, the ability of betaine:trifluoroacetamide at a 1:2 mol:mol ratio and diluted with 5% ethanol to maintain viral particle intactness makes this composition particularly useful for use in preparing viral samples for RNA analysis, for example, capturing virus particles on a bead or membrane (such as a membrane of a lateral flow device), as discussed herein.

A related aspect provides a vaccine composition for use in preventing a viral disease, which composition comprises an inactivated virus obtainable by a use as defined herein. The viral disease may be COVID-19 and the inactivated virus may be an inactivated SARS-CoV-2.

In still another aspect, there is provided a composition comprising:
a quaternary ammonium compound;
a halogenated organic compound; and
a solvent;

wherein the quaternary ammonium compound is present in the composition at a concentration in the range 0.1 mM to 400 mM;

wherein the halogenated organic compound is present in the composition at a concentration in the range 0.1 to 850 mM;

wherein the quaternary ammonium compound is a compound of Formula 1 or a salt thereof:

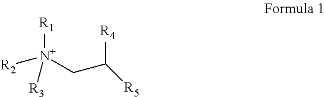

wherein:
R1, R2 and R3 are each independently selected from a methyl group and ethyl group;
R4 is H or OH;
R5 is selected from H, a halide, a carbonyl oxygen, a methyl group, and

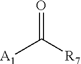

wherein:
   A1 is selected from CH₂, O and S;
   R7 is selected from OH, an unsubstituted C1 to C3 alkyl group, an alkyl carboxylic acid group having 1 to 3 carbon atoms, and a substituted C1 to C3 alkyl group bearing one or more substituents selected from hydroxyl and halide groups;
   with the proviso that, when R5 is H or a methyl group, R4 is OH;

and wherein the halogenated organic compound is a compound of Formula 2:

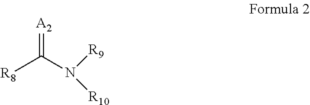

wherein:
A2 is O or S;
R8 is a substituted C1 to C12 alkyl group bearing one or more substituents selected from fluoro, chloro, bromo, and iodo; and
   i) R9 and R10 are each independently selected from H, an unsubstituted C1 to C12 alkyl group; an unsubstituted alkyl ester group having 2 to 12 carbon atoms; an alkyl ester group having 2 to 12 carbon atoms and bearing one or more substituents selected from fluoro, chloro, bromo, iodo, and hydroxyl; and a substituted C1 to C12 alkyl group bearing one or more substituents selected from fluoro, chloro, bromo, iodo, and hydroxyl; or
   ii) R9 and R10 together form an imidazole group.

The composition may allow for inactivation of a virus, and may allow for analysis or identification of the virus without the need to separate the virus from the composition. The composition may provide an adequate level of stabilisation to viral RNA, DNA or proteins to enable detection of the virus after a period of e.g. 72 hours. For example, stabilisation may be adequate to allow detection of the RNA, DNA or proteins of interest after storage under ambient conditions (e.g., a temperature of about 20° C.) for a period of 72 hours.

It is to be appreciated that the various features of the solvent and compounds of Formulae 1 and 2 described above with reference to the use aspect are also applicable to the composition aspect.

For example, the solvent may be a solvent of Formula 3, as discussed above.

The solvent may in particular comprise diethylene glycol, triethylene glycol, and/or a polyethylene glycol. A preferred polyethylene glycol is tetraethylene glycol. These solvents are readily available and have good safety profiles. More generally, the use of a non-aqueous solvent may allow for improved stabilisation of biomolecules compared to the use of an aqueous solvent.

The composition may further comprise an inactivated virus.

The composition may include a biological sample. The biological sample may be any of the biological samples identified above, in particular breath condensate.

A related aspect provides a kit, comprising:
a container, containing the composition; and
a swab. The kit is useful for sample collection and transport, particularly in the context of diagnostic testing.

The nature of the swab is not particularly limited. For example, the swab may be a nasal swab, nasopharyngeal swab or oropharyngeal swab.

The kit may further comprise a lateral flow device. The lateral flow device may be a nucleic acid lateral flow, NALF, device. The lateral flow device may be a nucleic acid lateral flow immunoassay, NALFIA, device. IIIustrative lateral flow devices are discussed in detail in e.g. Jauset-Rubio, M., Svobodová, M., Mairal, T. et al. Ultrasensitive, rapid and inexpensive detection of DNA using paper based lateral flow assay. Sci Rep 6, 37732 (2016).

The kit may include packaging for the container and the swab. The packaging may be, for example, a padded envelope.

In another aspect, there is provided a method for detecting an analyte in a sample, wherein the analyte is a biomolecule selected from an RNA and a DNA. The method comprises: using a deep eutectic solvent, preparing the sample for nucleic acid analysis; and performing nucleic acid analysis to detect the analyte. The nucleic acid analysis is carried out in the presence of crude cellular components. In other words, the analysis is performed on an unpurified or partially purified sample. This may allow the analysis to be performed more quickly and to use less resources than conventional methods which require purification. At the same time, preparing the solvent for analysis using a deep eutectic solvent may mitigate or overcome drawbacks associated with the use of unpurified samples.

For example, the deep eutectic solvent may inactivate a virus or other pathogen in the sample. The deep eutectic solvent may be as described above with reference to the use and composition aspects. The deep eutectic solvent may stabilise the analyte, in other words, protect the analyte from degradation. The deep eutectic solvent may reduce the binding of protein or small molecules to the analyte, thereby allowing for more reliable detection of the analyte.

Using the deep eutectic solvent typically comprises contacting the sample with the deep eutectic solvent. If the sample comprises a fluid, then the sample may be mixed with the deep eutectic solvent. If the sample is a solid sample, or is collected on a solid matrix such as a swab, then the sample may be immersed in the deep eutectic solvent.

The sample may comprise unpurified or partially-purified RNA and/or DNA. In other words, the sample may have a ratio of optical density at 260 nm to optical density at 280 nm of less than 1.8. The ratio of absorbances of a sample at 260 nm and 280 nm provides a measure of the degree of purity of nucleic acids in the sample. Optical density may be measured by spectrophotometry.

The crude cellular components may comprise proteins. The method may include lysing cells in the sample, and the crude cellular components may comprise cell debris and small molecules.

The analyte may be an RNA. RNA is particularly susceptible to degradation, and deep eutectic solvents are effective for inhibiting the degradation of nucleic acids.

The deep eutectic solvent may remove complexed or bound proteins from the analyte, e.g. RNA. This may allow for more reliable detection of the analyte. Complexed or bound proteins can interfere with the detection of nucleic acids.

The method may further comprise, before preparing the sample, performing an RNase inactivation step. This may help to prevent degradation of the analyte.

The method may further comprise, prior to performing the nucleic acid analysis, treating the sample with one or more enzymes selected from proteases, nucleases, lipases, and amylases. Treating the sample with an enzyme may reduce the viscosity of the sample. This may allow the sample to be manipulated more easily or more reliably, e.g. by pipetting. Treating the sample with an enzyme may break down a sample matrix. Examples of sample matrices include sputum mucin, cell membrane, viral envelopes, and viral capsids. Breaking down the sample matrix may improve the sensitivity of the nucleic acid analysis.

The sample may include eukaryotic cells. The method may further comprise lysing the eukaryotic cells to release cell contents into an in vitro environment comprising an aqueous buffer.

The sample may include a cell infected with a virus, wherein the virus is SARS-CoV-2.

The nature of the sample is not particularly limited. The sample may be a clinical sample, collected from a patient. The sample may for example include saliva; sputum; a nasal sample, a nasopharanyngeal sample; an oronasopharangyl sample; mucus; nasal mucus; or a droplet derived from a sneeze, cough, or expired breath (e.g., breath condensate as discussed with reference to the use aspect).

The nature of the nucleic acid analysis is also not particularly limited. The analysis may be qualitative or quantitative. Examples of nucleic acid analyses include LAMP, PCR, qPCR, RT-PCR, and RT-qPCR.

The analysis may comprise a lateral flow assay, using a lateral flow device. The lateral flow device may be a nucleic acid lateral flow, NALF, device. The lateral flow device may be a nucleic acid lateral flow immunoassay, NALFIA, device.

The sample may be partially-purified. For example, the method may further comprise concentrating a virus and/or cell in the sample.

The concentrating may comprise contacting the sample with a bead, particle, or membrane having a surface configured to bind to the virus and/or cell. The membrane may be a membrane of a lateral flow test device.

The bead or particle may have a diameter of less than 50 μm, optionally less than 40 μm, and further optionally in the range 4 to 30 μm in diameter. Beads or particles having diameters within these ranges may be conveniently handled using microfluidic systems of the type commonly used for single cell analysis.

The surface of the bead or particle may be configured to bind viruses, nucleic acids, or cells non-specifically. For example, the bead or particle may have a surface coating selected from a glycoprotein, heparin, and/or sialic acid.

Alternatively, the bead or particle may have a surface which is configured to bind selectively a target. For example, the coating may comprise an antibody, or a binding target for a virus. In implementations where the virus is SARS-CoV-2, the surface may comprise an ACE2 protein.

An oligonucleotide tag for identifying a batch of beads or particles may be conjugated to the surface of the bead or particle. The tag may allow identification of a particular sample based on the results of the nucleic acid analysis. This may be useful in implementations where the method is used for diagnostic purposes, or may allow for multiplexing of analyses.

The bead or particle has a streptavidin coating. A streptavidin coating is useful for binding biotinylated molecules to the bead or coating. In implementations where an oligonucleotide tag is used, the oligonucleotide may be biotinylated and attached to the particle by the streptavidin coating. Alternatively or additionally, an antibody or other protein for binding a virus may be biotinylated and attached to the bead or particle by the streptavidin coating.

As an alternative to using a bead or particle, concentrating the virus may comprise capturing the virus using a cell which expresses a virus binding partner on a surface of the cell. For example, the virus binding partner may be an ACE2 protein and the virus is a coronavirus. The cell may be a living cell, or may be a fixed cell. The fixed cell may be fixed using a deep eutectic solvent.

The cell may be a barcoded cell. Various methods for cell barcoding are known in the art. Barcoding the cell may allow identification of a particular sample based on the results of the nucleic acid analysis.

The method may further comprise treating the cell and virus with a reagent to enhance and stabilize attachment of the virus to the surface of the cell, wherein the reagent is selected from formaldehyde, a deep eutectic solvent, an alcohol, a metal salt, an acid, and mixtures thereof.

The nucleic acid analysis may comprise single-cell DNA-seq or single-cell RNA-seq, in particular in implementations where the method includes concentrating a virus and/or cell in the sample.

The deep eutectic solvent may be trimethylglycine:trifluoroacetamide. The molar ratio of the N,N,N-trimethylglycine to the trifluoroacetamide may be 1:2. The deep eutectic solvent is present in a mixture further comprising solvent, for example a solvent of Formula 3, and in particular a polyethylene glycol. The deep eutectic solvent may be present in a mixture further comprising formic acid at a concentration in the range 50 mM to 3.6 M, or acetic acid at a concentration of 100 mM to 13 M.

### BRIEF DESCRIPTION OF THE DRAWINGS

To assist understanding of embodiments of the present disclosure and to show how such embodiments may be put into effect, reference is made, by way of example only, to the accompanying drawings in which:

FIG. 1 is a plot showing the viscosity of Betaine:Trifluoroacetamide (1:2 mol:mol) diluted with varying amounts of a polyethylene glycol, PEG 200.

FIG. 2 is a photograph of a gel obtained in Example 36, illustrating RNA stabilisation in mouse liver incubated in various compositions for 5 days at 20° C. Lane 1: 100% Betaine:Trifluoroacetamide (1:2 mol:mol), Lane 2: 10% Betaine:Trifluoroacetamide (1:2 mol:mol)/90% PEG 200, Lane: 3 Positive control.

FIG. 3 is a photograph of a gel obtained in Example 37, showing RNA stabilisation in MKN Cell pellets stored for 20 hours at 60° C. in various compositions. Lane 1: 100% Betaine:Trifluoroacetamide (1:2 mol:mol). Lane 2: 50% Betaine:Trifluoroacetamide (1:2 mol:mol)/50% PEG 200. Lane 3: 30% Betaine:Trifluoroacetamide (1:2 mol:mol)/70% PEG 200. Lane 4: 10% Betaine:Trifluoroacetamide (1:2 mol:mol)/90% PEG 200.

FIG. 4 is a photograph of a gel obtained in Example 39, showing RNA stabilisation in MKN tissue culture cells incubated for 13 days at 37° C. Lane 1: 100% Betaine:Trifluoroacetamide (1:2 mol:mol): Lanes 2-9. Betaine:Trifluoroacetamide (1:2 mol:mol) with 12% (Lane 2), 23% (Lane 3), 34% (Lane 4), 42% (Lane 5), 54% (Lane 6), 64% (Lane 7), 74% (Lane 8), and 84% (Lane 9) PEG 200 final concentration by volume.

FIG. 5 is photograph of a gel obtained in Example 40, showing the effect of various compositions on PCR activity. Lanes 1-5; Betaine:Trifluoroacetamide (1:2 mol:mol), Lanes 6-10; 50% Betaine:Trifluoroacetamide (1:2 mol:mol)/50% PEG 200, Lanes 11-15; 10% Betaine:Trifluoroacetamide (1:2 mol:mol)/90% PEG 200 each tested at 0.05 (Lanes 1, 6, 11), 0.1 (Lanes 2, 7, 12), 0.5 (Lanes 3, 8, 13), 1 (Lanes 4, 9, 14) and 5% (Lanes 5, 10, 15) final (vol) concentration.

FIG. 6A is a photograph showing the effect of varying amounts of composition on IgG/IgM Serology lateral flow Rapid Test (SARS-CoV-2 IgG/IgM Rapid Test (Ref: Acon, China L031-11711); Lane 1. positive control, Lane 2. 19%, Lane 3. 32% (final vol) of 10% Betaine:Trifluoroacetamide (1:2 mol:mol)/90% PEG 200.

FIG. 6B is a photograph showing the effect of varying amounts of composition on Antigenic lateral flow Rapid Test (SARS-CoV-2 Rapid Antigen Test Nasal, Ref: Roche, Germany 9901-NCOV-03G). Lane 1: positive control. Lane 2 5%, Lane 3. 10%, Lane 4. 20% (final vol) of a mixture comprising 10% Betaine:Trifluoroacetamide (1:2 mol:mol)/90% PEG 200.

FIG. 7 is a photograph showing the effect of different types of 10% Betaine:Trifluoroacetamide (1:2 mol:mol)/90% Glycol mixtures on an Antigenic lateral flow Rapid Test (SARS-CoV-2 Rapid Antigen Test Nasal, Ref: Roche, Germany 9901-NCOV-03G). Lane 1. 10% Betaine:Trifluoroacetamide (1:2 mol:mol)/90% Tetraethylene glycol, Lane 2. 10% Betaine:Trifluoroacetamide (1:2 mol:mol)/90% Tetraethylene glycol monomethyl ether, and Lane 3. 10% Betaine:Trifluoroacetamide (1:2 mol:mol)/90% PEG 200.

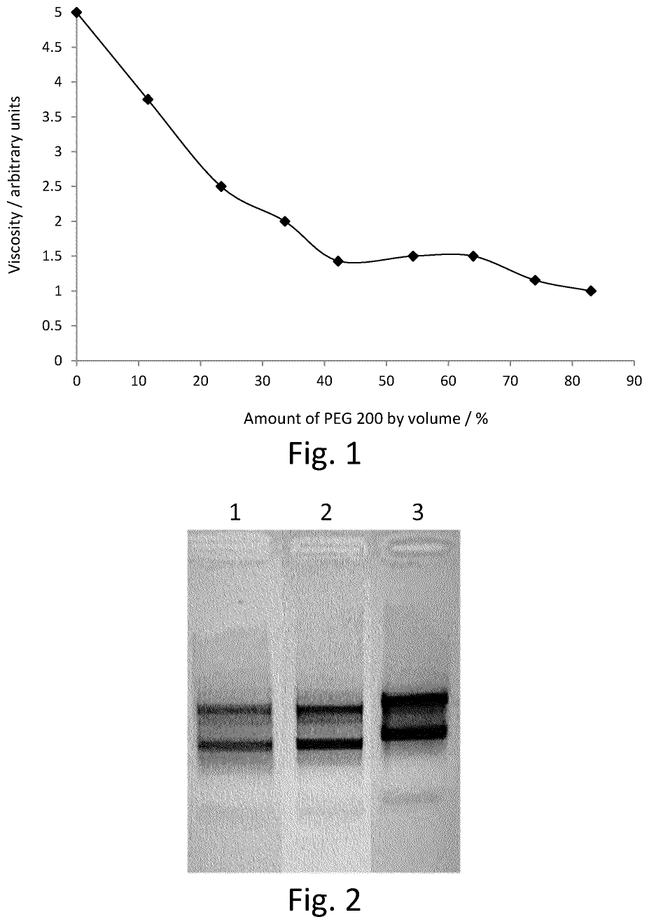

### DETAILED DESCRIPTION

The verb ‘to comprise’ is used herein as shorthand for ‘to include or to consist of’. In other words, although the verb ‘to comprise’ is intended to be an open term, the replacement of this term with the closed term ‘to consist of’ is explicitly contemplated, particularly where used in connection with chemical compositions.

All viscosity measurements reported herein are measured using a falling ball viscometer at a temperature of 20° C. at atmospheric pressure and under unit gravity. In particular, viscosity may be measured in accordance with the protocol described on page 567, section V of Schaschke, International Review of Chemical Engineering, Vol. 2, N. 5, pp. 564-576.

Unless otherwise stated, all percentages are by volume, based on the volume of the relevant mixture. Volumes are measured at a temperature of 20° C. and at atmospheric pressure (1 atm).

The term “about” where used in connection with a numeral contemplates a variance of ±10 As used herein, “betaine” refers to N,N,N-trimethylglycine.

Examples of biomolecules include RNA, DNA, proteins, phosphoproteins, lipids and carbohydrates.

A biomolecule is stabilised by a compound in a composition if degradation of the biomolecule is inhibited compared to a comparative composition which lacks the compound, but is otherwise identical. Methods for determining biomolecule stabilisation have been described in U.S. Pat. No. 9,696,247 to Goldsborough and Bates.

It will be appreciated that some compounds disclosed herein may be ionisable, i.e. some compounds may be weak acids, weak bases, or ampholytes. Representations of the free forms of ionisable compounds are intended to encompass the corresponding ionised forms, and vice versa. For example, representations of carboxylic acid groups encompass the corresponding carboxylate groups; and representations of carboxylate groups are intended to encompass the corresponding carboxylic acid.

The term “quaternary ammonium compound” as used herein refers in particular to a compound of Formula 1. The term “halogenated organic compound” refers in particular to a compound of Formula 2.

A “fluorinated organic compound” may be a compound of Formula 2, including at least one fluoro substituent.

The abbreviation “DES” refers to a deep eutectic solvent. DESs are mixtures of two or more components that when combined together have a eutectic point, which is the temperature of solidification or freezing (Fp). The eutectic point of the combined components is generally much lower than either of the components individually or at any other ratio of mixing and occurs at a single temperature without separation of the individual components on solidification.

The properties of DESs have been described in Abbott et al Chem. Commun. 7 (2003) 70-71; Abbott et al., J. Am. Chem. Soc. 126 (2004) 9142-9147; Imperato et al., Chem. Commun. 9 (2005) 1170-1172, Gore et al., Green Chem. 13 (2011) 1009-1013; Gorke et al Chem. Commun. 10 (2008) 1235-1237; Abbott et al, Aust. J. Chem. 62 (2009) 341-347; Choi et al. Plant Physiol. 156 (2011) 1701-1705; and reviewed by Zhang et al, Chem Soc Rev. 2012 Nov. 7; 41(21):7108-46.

Industrially, DESs have been used for electrochemical plating, mining applications and drill lubricants (US2009/0247432 A1), industrial enzyme applications (US2009/0117628 A1), preparation of inorganic compounds (Freudenmann et al., Angew. Chem., Int. Ed., (2011), 50, 11050-11060) or organic compounds (Gore et al. Green Chem., (2011), 13, 1009-1013), biological extractions (WO 2011/155829 A1), in electrochemistry as electrolytes for dye-sensitized solar cells and metal electropolishing (Jhong et al. Electrochem. Commun., (2009), 11, 209-211), electrodeposition (Gomez, et al., J. Electroanalytical Chem., (2011), 658, 18-24), purification of biodiesel (Shahbaz et al. Energy Fuels, (2011), 25, 2671-2678), solubilisation of drugs (Morrison et al. Int. J. Pharm., (2009), 378, 136-139), solubilisation of metal oxides (Abbott et al, J. Am. Chem. Soc., (2004), 126, 9142-9147) and solubilisation of CO2 (Li et al, J. Chem. Eng. Data, (2008), 53, 548-550).

DESs are not considered to be ionic liquids because: (i) they are not entirely composed of ionic species and (ii) they can also be obtained from non-ionic species, and (iii) they are mixtures and not compounds. As compared to the traditional ionic liquids, DESs have several advantages such as (1) low cost, (2) chemically inert to water, (3) easy to prepare by simply mixing two or more components, (4) most are biodegradable and non-toxic, (5) low volatility even when heated and (6) non-flammable. All DESs are liquids below 150° C. and many are liquid between room-temperature and 70° C., with a few notable examples that are liquid below 0° C.

The use of DESs to inhibit the degradation of biomolecules is disclosed in WO 2014/131906 A1. It has surprisingly been found that, although DESs are effective for stabilising RNA and proteins, certain DESs are also effective for inactivating viruses.

Even more surprisingly, inactivation and an acceptable level of biomolecule stabilisation may be achieved even when the DES mixture is diluted using a solvent to form a composition, even when the resulting composition is not necessarily itself a DES. A diluted composition may have reduced viscosity and thus, improved compatibility with liquid-handling robots. A diluted composition may have improved safety characteristics, allowing for handling by the general public outside of a laboratory. These characteristics are particularly relevant in the context of mass testing for infectious diseases.

It has been found that compositions comprising (a) quaternary ammonium compounds, particularly zwitterionic compounds, in combination with (b) halogenated organic compounds, particularly fluorinated organic compounds such as fluorinated acetamides, are capable of efficiently inactivating viruses such as SARS-CoV-2 virus and killing bacteria, fungi, parasites and pathogens, as well as stabilising biomolecules including RNA, DNA and proteins. These compositions may also act as fixatives, and may maintain cell and tissue structure.

The composition may be a DES. Alternatively, the composition may be solution of the quaternary ammonium compound and halogenated organic compound in an aqueous or non-aqueous solvent. In implementations where the composition is a solution, the stoichiometric ratio between the quaternary ammonium compound and halogenated organic compound is not particularly limited and is not necessarily a ratio which would form a DES in the absence of the aqueous or non-aqueous solvent.

Aqueous and non-aqueous dilutions can be made with either: (i) stoichiometric molar ratios of the quaternary ammonium and halogenated organic compound, as in for example Betaine:Trifluoroacetamide 1:2 mol:mol in the form of a Deep Eutectic Solvent, or alternatively (ii) non-stoichiometric ratios such as 400 mM Betaine and 850 mM Trifluoroacetamide.

The concentration of the quaternary ammonium compound in the non-aqueous or aqueous dilution may be in the range 0.1 mM to 8 M, optionally 1 mM to 8 M, preferably 10 to 600 mM, more preferably 40 to 500 mM and most preferably 0.1 mM to 400 mM.

The concentration of the halogenated organic compound may be in the range 0.1 mM to 8 M, optionally 1 mM to 8 M, more preferably 20 mM to 1.2 M, more preferably 80 mM to 1 M and most preferably 850 mM or less. Compositions which comprise the halogenated organic compound at a concentration of less than or equal to 850 mM, e.g., in the range 0.1 mM to 850 mM, optionally 1 mM to 850 mM may allow the inactivated virus to be analysed or detected without first separating the inactivated virus from the composition.

Particularly preferred are mixtures of zwitterionic quaternary ammonium compounds with fluorinated organic compounds, and as one specific example Betaine and Trifluoroacetamide.

By way of example, but without limitation, the quaternary ammonium compound of Formula 1 may be selected from: Choline nitrate, Choline tetrafluoroborate, Choline hydroxide, Choline bitartrate, Choline dihydrogen citrate, Choline p-toluenesulfonate, Choline bicarbonate, Choline chloride, Choline bromide, Choline iodide, Choline fluoride, Chlorocholine chloride, Bromocholine bromide, lodocholine iodide, Acetylcholine hydroxide, Acetylcholine bitartrate, Acetylcholine dihydrogen citrate, Acetylcholine p-toluenesulfonate, Acetylcholine bicarbonate, Acetylcholine chloride, Acetylcholine bromide, Acetylcholine iodide, Acetylcholine fluoride, Chloroacetylcholine chloride, Bromoacetylcholine bromide, lodoacetylcholine iodide, Butyrylcholine hydroxide, Butyrylcholine bitartrate, Butyrylcholine dihydrogen citrate, Butyrylcholine p-toluenesulfonate, Butyrylcholine bicarbonate, Butyrylcholine chloride, Butyrylcholine bromide, Butyrylcholine iodide, Butyrylcholine fluoride, ChloroButyrylcholine chloride, BromoButyrylcholine bromide, IodoButyrylcholine iodide, Acetylthiocholine chloride, L-Carnitine, D-Carnitine, Betaine, Sarcosine, Betaine HCl, Beta-methylcholine chloride, Choline citrate, and mixtures thereof.

In particular, the compound of Formula 1 may be Betaine. When betaine is in the form of a salt, the salt may be the hydrochloride salt.

Additional examples of compounds, not in accordance with Formula 1, which can be used instead of or in addition to a compound of Formula 1 include: Dimethylglycine, Glycine, Trimethylamine N-oxide, Cetyl betaine, Cetyltrimethylammonium fluoride, Cetyltrimethylammonium chloride, Cetyltrimethylammonium bromide, Lauryl betaine, N,N-Dimethylenethanolammonium chloride, N,N-diethyl ethanol ammonium chloride, Beta-methylcholine chloride, Phosphocholine chloride, Benzoylcholine chloride, Lauryl sulphobetaine, Benzyltrimethylammonium chloride, Methyltriphenylphosphonium chloride, Methyltriphenylphosphonium bromide, Methyltriphenylphosphonium iodide, Methyltriphenylphosphonium fluoride, N,N-diethylenethanol ammonium chloride, ethylammonium chloride, Tetramethylammonium chloride, Tetramethylammonium bromide, Tetramethylammonium iodide, Tetramethylammonium fluoride, Tetraethylammonium chloride, Tetraethylammonium bromide, Tetraethylammonium iodide, Tetraethylammonium fluoride, Tetrabutylammonium chloride, Tetrabutylammonium bromide, Tetrabutylammonium iodide, Tetrabutylammonium fluoride, sarcosine, and/or (2-chloroethyl) trimethylammonium chloride.

It is preferable to have one Ammonium group and one Carboxylate group in the quaternary ammonium compound, and for the molecule to be zwitterionic. One example the compound is Betaine.

The quaternary ammonium compound may have a molecular weight of less than 600 Daltons, more preferably less than 400 Daltons, more preferably less than 200 Daltons and most preferably in the range 60 to 150 Daltons.

The halogenated organic compound of Formula 2 may be selected from: Fluoroacetamide, Difluoroacetamide, Trifluoroacetamide, Chlorofluoroacetamide, Chlorodifluoroacetamide, Chloroacetamide, Dichloroacetamide, Dichlorofluoroacetamide, Trichloroacetamide, Bromoacetamide, Dibromoacetamide, Tribromoacetamide, Bromofluoroacetamide, Bromodifluoroacetamide, Bromochlorofluoroacetamide, lodoacetamide, Diiodoacetamide, Triiodoacetamide, 2,2-difluoropropanamide, 2-fluoropropanamide, 2-methyl-2-fluoropropanamide, 2,2-difluorobutanamide, 2-fluorobutanamide, 2-ethyl-2-fluorobutanamide, 2,2-difluoropentanamide, 2-fluoropentanamide, 2Propyl-2-fluoropentanamide, 3-Fluoropropanamide, 2,3-Difluoropropionamide, 3,3-Difluoropropionamide, 3,3,3-Trifluoropropionamide, 2-Fluoro-3,3,3-trifluoropropionamide, 2-Chloro-3,3,3-trifluoropropionamide, 2,2-Chloro-3,3,3-trifluoropropionamide, 2-bromo-3,3,3-trifluoropropionamide, 2,2-Bromo-3,3,3-trifluoropropionamide, Pentafluoropropionamide, Heptafluorobutanamide, 1-(Trifluoroacetyl)imidazole, N,O-Bis(trifluoroacetyl)hydroxylamine, Bistrifluoroacetamide, N-Methyl-fluoroacetamide, N-Methyl-difluoroacetamide, N-Methyl-trifluoroacetamide, N-Methyl-chlorofluoroacetamide, N-Methyl-chlorodifluoroacetamide, N-Methyl-chloroacetamide, N-Methyl-dichloroacetamide, N-Methyl-dichlorofluoroacetamide, N-Methyl-trichloroacetamide, N-Methyl-bromoacetamide, N-Methyl-dibromoacetamide, N-Methyl-tribromoacetamide, N-Methyl-bromofluoroacetamide, N-Methyl-bromodifluoroacetamide, N-Methyl-bromochlorofluoroacetamide, N-Methyl-iodoacetamide, N-Methyl-diiodoacetamide, N-Methyl-triiodoacetamide, N-Methyl-2,2-difluoropropanamide, N-Methyl-2-fluoropropanamide, N-Methyl-2-methyl-2-fluoropropanamide, N-Methyl-2,2-difluorobutanamide, N-Methyl-2-fluorobutanamide, N-Methyl-2diethyl-2-fluorobutanamide, N-Methyl-2,2-difluoropentanamide, N-Methyl-2-fluoropentanamide, N-Methyl-2-propyl-2-fluoropentanamide, N-Methyl-3-fluoropropionamide, N-Methyl-2,3-difluoropropionamide, N-Methyl-3,3-difluoropropionamide, N-Methyl-3,3,3-trifluoropropionamide, N-Methyl-2-fluoro-3,3,3-trifluoropropionamide, N-Methyl-2-chloro-3,3,3-trifluoropropionamide, N-Methyl-2,2-chloro-3,3,3-trifluoropropionamide, N-Methyl-2-bromo-3,3,3-trifluoropropionamide, N-Methyl-2,2-bromo-3,3,3-trifluoropropionamide, N-Methyl-pentafluoropropionamide, N-Methyl-heptafluorobutyramide, N,N-Dimethyl-2,2,2-trifluoroacetamide, N-Ethyl-2,2,2-trifluoroacetamide, N,N-Diethyl-2,2,2-trifluoroacetamide, N-(Hydroxymethyl)Trifluoroacetamide, Ethyltrifluoroacetamide, and mixtures thereof.

The halogenated organic compound may in particular be selected from: difluoroacetamide, difluorothioacetamide, trifluoroacetamide, and trifluorothioacetamide.

The compound may include at least 1 Fluorine atom, and may include a Fluoromethyl group. One illustrative example of such a compound is Fluoroacetamide.

The compound may include at least 2 Fluorine atoms in the halogenated organic compound, and may for example include a Difluoromethyl group. One specific example is Difluoroacetamide.

Preferably, the compound includes at least 3 Fluorine atoms, and may for example include a Trifluoromethyl group. One, two or more trifluoromethyl groups may be present, with a single Trifluoromethyl group being preferred. A particularly preferred compound is Trifluoroacetamide.

The halogenated organic molecule may have a molecular weight of less than 400 Daltons, more preferably less than 300 Daltons, more preferably less than 200 Daltons and most preferably in the range 60 to 150 Daltons.

Particularly preferably, the composition comprises N,N,N-trimethylglycine as the quaternary ammonium compound, and trifluoroacetamide or trifluorothioacetamide as the halogenated organic compound. Trifluoroacetamide is preferred over trifluorothioacetamide because the latter has a strong odour. The composition may further comprise a polyethylene glycol as a solvent.

In some implementations, the composition comprising the quaternary ammonium compound and halogenated organic compound further comprises a solvent. The term “diluent” is used herein to refer to a solvent, optionally further comprising one or more additives.

The diluent may comprise an aqueous or non-aqueous solvent. Aqueous and non-aqueous solvents are both suitable as diluents for disinfectants and antiseptics, for inactivating viruses and killing bacteria, fungi, parasites including pathogens. Non-aqueous solvents, in particular Polyethylene glycols such as PEG 200, may be preferred when the preservation and stabilisation of biomolecules, for example RNA, DNA, proteins or phosphoproteins, is important, for example when the inactivated virus is to be subsequently analysed or identified.

The diluent may further comprise one or more additives, e.g., solutes. The nature of the additives is not particularly limited and may be selected as appropriate. The solutes may comprise one or more buffers, detergents, salts, proteins, nucleic acids, stabilisers, preservatives, and/or chelators. Solutes may in particular be incorporated in implementations in which the solvent comprises water.

IIIustrative examples of additives are: tris(hydroxymethyl)aminomethane (“Tris”) and salts thereof, such as Tris-HCl, piperazine-N,N′-bis(2-ethanesulfonic acid) (“PIPES”), 2-(N-morpholino)ethanesulfonic acid (“MES”), 4-(2-hydroxyethyl)-1-piperazineethanesulfonic acid (“HEPES”), 3-(N-morpholino)propanesulfonic acid (“MOPS”), β-Hydroxy-4-morpholinepropanesulfonic acid (“MOPSO”), N-cyclohexyl-3-aminopropanesulfonic acid (“CAPS”), 3-(Cyclohexylamino)-2-hydroxy-1-propanesulfonic acid (“CAPSO”), BIPES, phosphate, imidazole, 1,2-bis(o-aminophenoxy)ethane-N,N,N′,N′-tetraacetic acid (“BAPTA”), ethylenediaminetetraacetic acid (“EDTA”), egtazic acid (“EGTA”), citric acid, D-Penicillamine, Urea, a Quaternary ammonium, a salt such as Ammonium sulphate, Ammonium chloride, Ammonium thiosulphate, Sodium dodecyl sulphate, Sodium lauryl sulphate, Sodium Benzoate, Sodium p-toluenesulphonic acid, Glycine, Dimethylglycine, Sarcosine, Guanidine, Guanidine HCl, Guanidine isothiocyanate, protein precipitant such as Trichloroacetic acid, Sulphosalicyclic acid, Zinc salt such as Zinc acetate, Zinc EDTA, Zinc phosphate, Zinc Trifluoroacetate, Zinc citrate, Zinc p-Toluenesulfonate, Zinc gluconate, Zinc chloride and/or Zinc sulphate, Molecular sieves 4 A, silica gel, CaCl2), LiCl, Ribitol, Rhamnose, Trehalose, D-Sorbitol, L-Sorbitol, Sorbose, Xylitol, Glucose, Sucrose, Lactose, Fructose, Maltose, Mannose, Mannitol, Arabinose, Galactose, Raffinose, Inositol, Erythritol, Xylose, Polyacrylamide, a high molecular weight polysaccharide such as Glucomannan, Cellulose, Starch, Inulin, Amylose, a gum such as Xanthan, Arabic or Guar, a protein such as Albumin, Casein, Haemoglobin, an antibody and/or an enzyme, an ionic or non-ionic detergent such as SDS, sodium lauryl sulphate, cetyltetrabutylammonium bromide, tetrabutylammonium bromide, saponin, sodium deoxycholate, branched Octylphenoxy poly(ethyleneoxy)ethanol (commercially available under the trade name “IGEPAL-CA630”), Polyoxyethylene lauryl ethers (such as those commercially available under the trade names “Brij-35” and “Brij-58”), Polyglycol ether (nonionic) surfactants such as NP-40, Polyethylene glycol p-(1,1,3,3-tetramethylbutyl)-phenyl ethers (e.g., Triton X-100, Triton X-114), polysorbates such as polysorbate 20 and polysorbate-80 (commercially available under the trade names “Tween-20” and “Tween-80”, respectively), Octyl beta glucoside, CHAPS, Solanine, natural water soluble Polymers such as pectins, xanthans, chitin, chitosan, dextran, ficoll, carrageenan, cellulose ethers, hyaluronic acid, albumin, gelatin, disaccharides, polysaccharides, starch and starch like derivatives, and, bulking agents, or fillers.

The composition may have a pH in the range of pH 2 to 13, more preferably pH 3 to 12, more preferably pH 4 to 11, more preferably pH 5 to 10, more preferably pH 6 to 9, more preferably pH 6 to 8, and most preferably pH 6 to 7.

It will be apparent to one skilled in the art that many compounds that dissolve into water to make an aqueous solution can also dissolve into non-aqueous liquids such as PEG 200, DMSO, alcohols or glycerol to produce a non-aqueous solution. The additives identified above may be mixed with a non-aqueous solvent instead of or in addition to water.

IIIustrative examples of non-aqueous solvents include: dimethyl sulfoxide (“DMSO”), Acetone, Dihydroxyacetone, Formamide, Dimethylformamide, 1,3-Diamino-acetone, Formaldehyde, Glutaraldehyde, an alcohol such as Pentanol, n-Butanol, isobutanol, tert-butanol, Propanol, Ethanol, Methanol, 1,2-propanediol, 1,3-propanediol, 1,4-Butanediol, 1,5-Pentanediol, 1,6-Hexanediol, 1,8-Octanediol, 1,12-Dodecanediol, Cresol, Tetramethyl urea, Imidazole, 1-Methylimidazole, 1-Ethylimidazole, 1-Benzylimidazole, 4-Methylimidazole, N-Methylpyrrolidone, N-Ethylpyrrolidone, N-Benzylpyrrolidone, Beta-mercaptoethanol, Ethyl carbonate, Phenol, an oil, a wax, liquid Paraffin, a Carboxylic acid such as Formic, Acetic, Propanoic, Butanoic, Lactic, Polyacrylic acid, Citric acids and halogenated derivatives thereof (for example, a derivative selected from fluorocitric acid, chloro citric acid, bromo citric acid), Glycerol, Ethylene glycol, Diethylene glycol, Triethylene glycol, Polyethylene glycol (“PEG”) with a molecular weight (“MW”) of 150 to 400,000 Daltons such as PEG 200, PEG 300, PEG 400, PEG 500 and PEG 600, Diethyleneglycol monoethylether acetate, polyethylene glycol methyl ether, polyvinyl pyrrolidone, polyvinyl alcohol, polyethylene oxide, hydroxypropylcellulose, polyvinyl pyrrolidone-vinyl acetate and/or N-(2-hydroxypropyl) methacrylamide.

Further examples of non-aqueous solvents include polypropylene glycol; polyvinyl butyral; polyvinyl esters such as polyvinyl acetate; polyacrylic acid; and copolymers such as poly(ethylene-vinylacetate). These compounds may have molecular weights in the range of 150 to 400,000 Da, in particular 150 to 600 Da.

The compositions provided herein may be useful as an inactivator of viruses and viral particles. The compositions may further act as a stabiliser of viral nucleic acids such as RNA and DNA, and/or other biomolecules such as proteins.

The virus may, for example, be:
(i) a dsDNA virus, e.g. an adenovirus, herpesvirus, Poxvirus;
(ii) an ssDNAvirus e.g. a Parvovirus;
(iii) a dsRNA virus, e.g. a Reovirus;
(iv) a positive strand ssRNA virus, e.g. a Coronavirus, a Picornavirus, a Togavirus;
(v) a negative strand ssRNA virus, e.g. an Orthomyxovirus, Rhabdovirus),
(vi) a DNA intermediate ssRNA virus e.g. a retrovirus);
(vii) an RNA intermediate dsDNA virus e.g., a Hepadnaviruses; or
(viii) a bacteriophage, such as M13.

More specific examples of viruses which may be inactivated, or inactivated and stabilised, by the present compounds and compositions include: HPV, HIV, HBV, HCV, Foot and Mouth Disease Virus, Influenza, Coronavirus such as SARS including MERS-CoV, SARS-CoV-1 and SARS-CoV-2, Zika, Rift Valley Disease Virus, BVDV, Vaccinia, Polio, Marburg virus, Lassa virus, Hantavirus, Ebola, and/or West Nile Virus, Norwalk, Rotavirus, Rabies, Influenza, Yellow Fever virus, Hepatitis A, C (HCV) and E virus, Dengue fever virus and plant RNA viruses and viroids.

The compositions may be used for inactivating and/or stabilising a bacteriophage. The bacteriophage may be a single stranded RNA bacteriophage, such as those of the genus Levivirus e.g. the Enterobacteria phage MS2, or of the genus Allolevivirus e.g. the Enterobacteria phage Qβ. The bacteriophage may be a double stranded RNA bacteriophage such as Cystovirus including Pseudomonas phage Φ6, a single-stranded DNA bacteriophage such as M13, or a double-stranded bacteriophage such as T4. Other types of phage such as those used as internal RNA controls for diagnostic applications such as those used in Armored RNA® (Ambion) may also be inactivated and/or stabilised.

The present compounds and compositions may also be used for stabilising internal control RNA or DNA sequences for use in diagnostic kits, by mixing the compound or composition with a nucleic acid.

Example uses of the present compositions include:
(i) preserving biomolecules, in particular RNA, DNA and proteins,
(ii) the inactivation and/or killing of viruses, bacteria, fungi and parasites including pathogens,
(iii) the inactivation and/or killing of viruses, bacteria, fungi and parasites including pathogens in a biological sample,
(iv) preserving biomolecules, in particular RNA, DNA and proteins whilst inactivating and/or killing viruses, bacteria, fungi and parasites including pathogens in a biological sample,
(v) the disinfection of a non-biological surface,
(vi) disinfection of a medical device,
(vii) an antiseptic for treating an infection in a living organism including plants, animals or humans,
(viii) the suppression of microbial growth including biofilms on a non-biological surface,
(ix) the disinfection of a non-biological sample,
(x) the treatment of a biological sample.

The nature of the sample to be treated with the present compounds and compositions is not particularly limited. IIIustrative examples of biological samples include:
(a) liquid specimens such as blood, plasma, serum, cerebral spinal fluid (CSF), saliva, sputum, bronchoalveolar lavage (BAL), breath vapour and aerosol condensate, amniotic fluid, milk and urine,
(b) solid specimens such as body tissues (liver, spleen, brain, muscle, heart, oesophagus, testis, ovaries, thymus, kidneys, skin, intestine, pancreas, adrenal glands, lungs, bone and bone marrow),
(c) clinical or medical specimens such as a prostate, breast or a cancer sample, tumour or biopsy, including a FFPE sample, circulating tumourcells, blood, clinical swabs, nasal swabs, nasopharyngeal swabs, oropharyngeal swabs, saliva, sputum, breath condensate, breath droplet condensate, dried blood, exosome, microvesicles,
(d) animal tissues derived from biomedical research or fundamental biology (e.g. tissues from monkey, rat, mouse, Zebra fish, Xenopus, Drosophila, nematode, yeast),
(e) tissue and tissue culture cells used for drug discovery purposes,
(f) pathogenic and non-pathogenic microbes such as fungi, archaebacteria, gram-positive and gram-negative bacteria, including E. coli, Staphylococcus, Streptococcus, Mycobacterium, Pseudomonas and bacteria that cause Listeria, Shigella, Diphtheria, Tetanus, Syphilis, Chlamydia, Legionella, Listeria and leprosy,
(g) pathogenic or non-pathogenic viroids, bacteriophage or viruses that are found in a variety of biological samples such as bacteria, plants, human tissues, blood, serum, plasma, saliva, sputum and tissues, and clinical samples,
(h) plants such as the leaves, flowers, pollen, seeds, stems and roots of rice, maize, sorghum, palm, vines, tomato, wheat, barley, tobacco, sugar cane and Arabidopsis,
(i) potentially pathogenic material associated with bioterrorism threats such as anthrax that may or may not need to be transported from the discovery site to the testing facility,
(j) food samples that may for example contain food borne diseases,
(k) samples including swabs taken from food processing environments such as production line,
(l) environmental samples such as soil and
(m) samples such as cellular and non-cellular blood products, platelet cells, red blood cells, white blood cells, plasma, serum, viruses and purified nucleic acids including RNA and/or DNA for gene therapy or viral and/or bacterial vaccines, antibodies, enzymes, proteins such as growth hormones, that are designed for therapeutic use and clinical delivery by inhalation, injection, infusion, transfusion, perfusion, topical application and/or orally, particularly for removal of pathogen infectivity.

It should be noted that the sample may be a naturally-derived sample, or a chemically or enzymatically synthesised sample, such as in vitro transcribed RNA and modified RNA, DNA, PCR products, oligodeoxyribonucleotides and oligoribonucleotides, PNA and LNA.

Example use cases include: (i) improving biosafety by disinfecting samples including clinical specimens and in particular samples procured from COVID-19 patients such as blood, saliva and nasopharyngeal swabs, (ii) both inactivating pathogens and stabilising analyte molecules such as RNA, DNA, proteins and phosphoproteins for diagnostic purposes, (iii) disinfecting non-clinical samples such as medical equipment and materials that have been in contact with a COVID-19 patient such as intubation tubes and medical devices, (iv) as an antiseptic for treating an infection, (v) for inactivating viruses, killing bacteria and/or other pathogens for use as an inactivated virus, bacterial and/or pathogen vaccine, (vi) for removing infectious particles from blood products for therapeutic use, (vii) for removing infectivity from gene therapy particles such as viral gene therapy vectors, (viii) food safety testing environment to preserve samples and render them non-dangerous and (ix) animal, particularly livestock testing for zoonotic pathogens including influenzas and coronaviruses.

Provided is a method for inactivating pathogens with a mixture of a quaternary ammonium compound and a halogenated organic compound such as a fluorinated organic compound, and optionally, diluted in a solvent to form an inactivated pathogen composition.

Provided is a method for inactivating viruses in a virus-containing sample, which method comprises contacting the sample with a mixture of a quaternary ammonium compound and a halogenated organic compound such as a fluorinated organic compound and optionally, diluted in a solvent to form a non-infectious composition.

Provided is a method for killing bacteria in a bacteria-containing sample, which method comprises contacting the sample with a mixture of a quaternary ammonium compound and a halogenated organic compound such as a fluorinated organic compound and optionally, diluted in a solvent to form a disinfected composition.

Provided is a method for killing fungi in a fungi-containing sample, which method comprises contacting the sample with a mixture of a quaternary ammonium compound and a halogenated organic compound such as a fluorinated organic compound and optionally, diluted in a solvent to form a disinfected composition.

Provided is a method for killing parasites in a parasite-containing sample, which method comprises contacting the sample with a mixture of a quaternary ammonium compound and a halogenated organic compound such as a fluorinated organic compound and optionally, diluted in a solvent to form a disinfected composition.

Provided is a method for stabilising RNA in an RNA-containing sample, which method comprises contacting the sample with a mixture of a quaternary ammonium compound and a halogenated organic compound such as a fluorinated organic compound and optionally, diluted in a solvent to form a stabilised RNA-containing composition.

Provided is a method for stabilising DNA in a DNA-containing sample, which method comprises contacting the sample with a mixture of a quaternary ammonium compound and a halogenated organic compound such as fluorinated organic compound in an aqueous or non-aqueous solvent to form a stabilised DNA-containing composition.

Provided is a method for stabilising proteins including phosphoproteins in a peptide-containing sample, which method comprises contacting the sample with a mixture of a quaternary ammonium compound and a halogenated organic compound such as a fluorinated organic compound and optionally diluted in a solvent to form a stabilised protein-containing composition.

Provided is a kit for inactivating viruses and killing bacteria, fungi and parasites in a sample, which kit comprises at least one container containing a mixture of a quaternary ammonium compound and a halogenated organic compound such as fluorinated organic compounds and optionally, diluted in a solvent. The kit further comprising instructions for contacting the sample with a mixture of a quaternary ammonium compound and a halogenated organic compound such as a fluorinated organic compound and optionally, diluted in a solvent whereby the virus, bacteria, fungi and parasites in the sample are inactivated or killed.

Provided is a virus inactivating composition, which comprises a virus and a mixture of a quaternary ammonium compound and a halogenated organic compound such as a fluorinated organic compound and optionally, diluted in a solvent.

Provided is a bacteria killing composition, which comprises a bacteria and a mixture of a quaternary ammonium compound and a halogenated organic compound such as a fluorinated organic compound and optionally, diluted in a solvent.

Provided is a fungi killing composition, which comprises a fungi and a mixture of a quaternary ammonium compound and a halogenated organic compound such as a fluorinated organic compound and optionally, diluted in a solvent.

Provided is a parasite killing composition, which comprises a parasite and a mixture of a quaternary ammonium compound and a halogenated organic compound such as a fluorinated organic compound and optionally, diluted in a solvent.

Provided is a stabilised RNA-containing composition, which comprises RNA and a mixture of a quaternary ammonium compound and a halogenated organic compound such as a fluorinated organic compound and optionally, diluted in a solvent.

Provided is a stabilised DNA-containing composition, which comprises DNA and a mixture of a quaternary ammonium compound and a halogenated organic compound such as a fluorinated organic compound and optionally, diluted in a solvent.

Provided is a stabilised protein-containing composition, which comprises protein and a mixture of a quaternary ammonium compound and a halogenated organic compound such as a fluorinated organic compound and optionally, diluted in a solvent.

Provided is the use of a mixture of a quaternary ammonium compound and a halogenated organic compound such as a fluorinated organic compound and optionally, diluted in a solvent as a disinfectant of viruses, bacteria, fungi and parasites including pathogens.

Provided is the use of a mixture of a quaternary ammonium compound and a halogenated organic compound such as a fluorinated organic compound and optionally, diluted in a solvent as a disinfectant of viruses, bacteria, fungi and parasites including pathogens and a stabiliser of biomolecules including DNA, RNA, proteins and phosphoproteins.

Provided is the use a mixture of a mixture of a quaternary ammonium compound and a halogenated organic compound such as a fluorinated organic compound and optionally, diluted in a solvent as an antiseptic for viruses, bacteria, fungi and parasites including pathogens.

Provided is the use of a mixture of a quaternary ammonium compound and a halogenated organic compound such as a fluorinated organic compound and optionally, diluted in a solvent as a disinfectant of viruses, bacteria, fungi and/or other pathogens such as Leishmania, Trypanosoma and/or Plasmodium in saliva, sputum, clinical swab samples, plasma, serum, buffy coat and/or blood for subsequent pathogen diagnostic analysis including bacterial and/or viral diagnostics.

Methods for Detecting Analytes

Also described herein are methods to use non-purified or partially purified sources of nucleic acids for analytical, including diagnostic purposes. The nucleic acids can be derived from a variety of sources including pathogens such as viruses, bacteria, fungi and parasites; or host sources such as plants, animals, or human clinical samples. It will be understood that the approach to identifying viruses will also relate to identifying bacteria, particularly those that are related to pathogens.

Using RNA or DNA directly from a virus, a sample containing a virus or viruses, a cell, a sample containing a cell or cells, a virus and a cell, and/or a sample containing a virus or viruses and a cell or cells, without prior nucleic acid purification requires the researcher to overcome the considerable potential negative effects of using non-purified samples such as: (i) enzyme inhibitors, (ii) lower amounts of detectable nucleic acid, (iii) nucleic acid structure, (iv) non-desired proteins and RNA bound to the desired nucleic acid, (v) enzyme contamination particularly by nucleases such as ribonucleases, (vi) instability of the sample during storage and transport, and (vii) biosafety issues when handling infectious and pathogenic samples that have not been purified.

Using a deep eutectic solvent, “DES”, overcomes several of the issues encountered when working with non-purified samples such as those described above. As one example of a useful DES, it is known that the DES composition Trimethylglycine:Trifluoroacetamide (1:2 mol:mol) (i) inhibits nucleases including ribonucleases, (ii) reduces nucleic acid secondary structure, (iii) and inactivates viruses, kills bacteria and knocks-down yeast. These features make the use of DES useful when handling non-purified samples especially when they contain nucleases, complex structure and infectious materials. As one part of this invention we describe the use of DES for improving the handling, safety, storage and logistics of samples in the context of carrying out an analytical test, in particular a diagnostic analytical test.

One of the purposes of the present methods is to provide a solution to the current significant problems associated with widespread virus testing and in particular SARS-CoV-2. It has been found that it possible to avoid the costly and time consuming step of RNA and DNA purification prior to the analytical step for example RT-PCR. It has been found that the use of Deep Eutectic Solvents is helpful in preparing the nucleic acid source for analysis.

A method for multiplexing diagnostic tests by leveraging scRNA-seq and scDNA-seq technology is also described.

It is generally assumed for historical and practical reasons that RNA and/or DNA purification; the process of removing other biological material from the desired RNA and/or DNA, is necessary in order to improve handling, storage and the downstream analytical detection of the analyte.

We have found that it is possible to use an unpurified sample as a source of the RNA and/or DNA used for the analytical test. In methods which use whole cells directly in a reverse transcription reaction, the eukaryotic cell, or in certain cases the nuclei, is lysed: the cell integrity is broken and the cell contents are released into an in vitro environment. The reaction components are then allowed, during an incubation period, to copy and/or react with the presence of the analyte RNA and/or DNA. This is commonly accompanied by heating the reaction or maintaining the reaction at a single or multiple temperatures as in reverse transcription. For example, the reaction may be heated or maintained at 25, 37, 42, 50, 55° C. or more for 15, 30, 45 or 60 minutes, or as one example for PCR analysis by cycling the temperature at 94, 55 and 72° C. for 10, 20, 30 or 60 seconds and repeating 1-30 or more times. The in vitro environment may comprise an aqueous buffer, and optionally one or more of the following: an enzyme, such as an RNA dependent DNA polymerase; ribonucleotide triphosphates; deoxyribonucleotide triphosphates; metal salts such as MgCl₂, MgSO₄ or MnCl₂; a buffer; a primer such as an oligonucleotide including oligo (dT) or RNA or DNA specific sequence with or without a bar-code; a Unique Molecular Identifier, “UMI”; a PCR primer site; dithiothreitol (“DTT”); a non-ionic detergent, such as Igepal-CA630 or Triton X-100; and a ribonuclease inhibitor such as RNasin.

Reverse transcription reactions may make use of an RNA-dependent DNA polymerase, a DNA-dependent DNA polymerase, or an RNA-dependent RNA polymerase.

Among the RNA-dependent DNA polymerases are Superscript™ II (MMLV reverse transcriptase Rnase H-), MMLV reverse transcriptase, HIV reverse transcriptase, AMV reverse transcriptase, RAV-2 reverse transcriptase, MarathonRT, Maxima H Minus (ThermoFisher). Enzymes which can be used successfully for polymerization/amplification include Superscript II, III or IV (ThermoFisher), MULV Rnase H.sup./ (Promega), MULV Rnase H. sup.-(Promega), Expand (Roche Molecular Biochemicals) and HIV-1 reverse transcriptase (Amersham Pharmacia). A mixture of Superscript II, III or IV and AMV (ThermoFisher) may also be used successfully.

Among the DNA-dependent DNA polymerases are DNA polymerase I;-Klenow fragment; T4 DNA polymerase; T7 DNA polymerase; Taq DNA polymerase, Tli DNA polymerase, Pfu DNA polymerase; Vent™ DNA polymerase; Deep Vent™ DNA polymerase; Bst DNA polymerase; Tth, Pfu Turbo™, Pfu(exo-), Pwo, Pyra™, Tfu, KlenTaq, Taq2000™, AmpliTaq Stoffel fragment, Sequenase™, Tma, Vent® (exo-), Deep Vent® (exo-).

Among the RNA-dependent RNA polymerases are Q beta replicase, and those derived from E. coli phage f2, R17, MS-2 or 06, or from a virus family selected from the bromoviridae, flaviviridae, picornaviridae, potyviridae, tobamovirus, tombusviridae, leviviruses, hepatitis C-like viruses, and picornaviruses or from polio virus, yellow fever virus, tobacco mosaic virus, brome mosaic virus, influenza virus, reovirus, myxovirus, rhabdovirus and paramyxovirus.

In the methods of detecting an analyte provided herein, at least one of the analytical steps is carried out in the presence of the crude non-purified cellular components, i.e. without prior RNA and/or DNA purification.

It is known that cellular components such as proteins or small molecules, such as heme, can change the behaviour of the RNA and/or DNA by, for example, interfering with the polymerase or another of the reaction components or by binding to the analyte RNA and/or DNA in such a way that the analytical process such as copying the RNA into cDNA is inhibited or altered in some way making the downstream analysis more difficult or even incorrect.

One well known example of inhibition is the strong inhibitory effect of heme from blood on DNA polymerase activity. By way of another example, it is well known that all types of cellular RNA including viral RNA, tRNA, mRNA and rRNA are not only intimately bound to cellular proteins such as ribonucleoproteins, but display highly structured secondary and tertiary sequences that can inhibit, for example, reverse transcriptases leading to an incomplete cDNA copy.

One of the reasons for commonly carrying out historical methods such as RNA purification with a chaotrope, for example guanidine thiocyanate, is to remove not only the ribonucleoproteins but also such highly structured sequences from the RNA, thereby improving the ‘template’ activity of the RNA with the reverse transcriptase. Similar issues are associated with non-purified DNA sequences, such as human genomic or mitochondrial DNA, as well as viral DNA sequences, which are commonly bound to nucleoproteins such as chromatin including histones and transcription factors.

It should be noted that viral single-stranded and double-stranded RNA and DNA within the viral particle is always found in the context of proteins that are more-or-less tightly bound to the nucleic acid. The cellular functions of these proteins are to allow tight nucleic acid packing, protect the nucleic acid from damaging environmental conditions, serve as a regulatory functions once released into a cell and serve as structural scaffolds for viral capsid formation. It is important to remove such proteins as well as nucleic acid secondary and tertiary structure, particularly for RNA analyte molecules which are always much more highly structured than DNA.

It will be apparent that if a particular RNA or DNA is not a good template for an enzyme or other assay components such as a hybridisation probe then the result is a false under-representation of the analyte concentration. From a practical stand-point, the target for the majority for transcriptomic studies, namely mRNA, is highly structured and complexed in vivo with various proteins and other RNA molecules as described previously. The natural function of some of these mRNA proteins is to regulate the post-transcriptional control of the mRNA, i.e. to control whether the mRNA will be translated into protein or not. Such post-transcriptional control is well known. For example, the cytokines IL2 and IL8 are complexed to proteins which control translation of the corresponding mRNA. It has been found that such proteins strongly affect the ability of reverse transcriptases to copy the mRNA into cDNA so that the final amount of detected mRNA is greatly under-represented compared with an RNA sample that has been purified prior to analysis. It is therefore important to reduce or remove such proteins complexing to the nucleic acid analytes, particularly for RNA analyte molecules or false results may be obtained.

Such issues are not simple or straightforward to overcome. Earlier approaches required careful case-by-case optimisation of reaction conditions and the addition of reagents such as a non-ionic detergent or a reducing agent, or heating or sonicating the sample to break down nucleic acid-protein complexes and thereby releasing the nucleic acid in a usable form.

The use of Deep Eutectic Solvents (“DES”) has been found to be useful to prepare the non or partially purified sample for nucleic acid analysis. The methods provided herein may remove complexed proteins and other contaminating and/or interfering molecules from the RNA using deep eutectic solvents.

Another issue related to RNA analysis in particular is that cells and biologically samples in general are a rich source of ribonucleases and other nucleases that frequently destroy the integrity of the analyte nucleic acid. Such nucleases are commonly removed during the RNA purification and wash steps thereby protecting the nucleic acid from subsequent degradation.

If the activity of such nucleases leads to the destruction of the analyte nucleic acid then there will be an under-representation of that sequence in the final results potentially leading to a false negative result. It is therefore important to protect the nucleic acid, particularly RNA molecules from enzymatic degradation when the sample has not been purified prior to analysis. Deep eutectic solvents are useful for protecting nucleic acids from degradation, as described in detail in WO 2014/131906 A1.

Deep Eutectic Solvents (DES) have been extensively described in WO2014/131906 A1. There is no particular limitation to the type of DES used in the present methods. Examples of these include, but are not limited to: Choline chloride:Trifluoroacetamide (1:2 mol:mol), Choline chloride:Glycerol (1:2 mol:mol), Choline chloride:Sorbitol (1:2 mol:mol), Choline chloride:Galactose (1:2 mol:mol), Choline chloride:Xylitol (1:2 mol:mol), Trimethylglycine:Urea (1:2 mol:mol), Trimethylglycine:Glycerol (1:2 mol:mol), Trimethylglycine:Sorbitol (1:2 mol:mol), Trimethylglycine:Galactose (1:2 mol:mol), Trimethylglycine:Xylitol (1:2 mol:mol) and/or Trimethylglycine:Trifluoroacetamide (1:2 mol:mol).

By way of example, but without limitation, the DES is made by mixing, or mixing and heating, one or more component(s) which may be chosen from the group: Choline nitrate, Choline tetrafluoroborate, Choline hydroxide, Choline bitartrate, Choline dihydrogen citrate, Choline p-toluenesulfonate, Choline bicarbonate, Choline chloride, Choline bromide, Choline iodide, Choline fluoride, Chlorocholine chloride, Bromocholine bromide, lodocholine iodide, Acetylcholine hydroxide, Acetylcholine bitartrate, Acetylcholine dihydrogen citrate, Acetylcholine p-toluenesulfonate, Acetylcholine bicarbonate, Acetylcholine chloride, Acetylcholine bromide, Acetylcholine iodide, Acetylcholine fluoride, Chloroacetylcholine chloride, Bromoacetylcholine bromide, lodoacetylcholine iodide, Butyrylcholine hydroxide, Butyrylcholine bitartrate, Butyrylcholine dihydrogen citrate, Butyrylcholine p-toluenesulfonate, Butyrylcholine bicarbonate, Butyrylcholine chloride, Butyrylcholine bromide, Butyrylcholine iodide, Butyrylcholine fluoride, ChloroButyrylcholine chloride, BromoButyrylcholine bromide, IodoButyrylcholine iodide, Acetylthiocholine chloride, L-Carnitine, D-Carnitine, Betaine, Sarcosine, Trimethylamine N-oxide, Betaine HCl, Cetyl betaine, Cetyltrimethylammonium fluoride, Cetyltrimethylammonium chloride, Cetyltrimethylammonium bromide, Lauryl betaine, N,N-Dimethylenethanolammonium chloride, N,N-diethyl ethanol ammonium chloride, Beta-methylcholine chloride, Phosphocholine chloride, Choline citrate, Benzoylcholine chloride, Lauryl sulphobetaine, Benzyltrimethylammonium chloride, Methyltriphenylphosphonium chloride, Methyltriphenylphosphonium bromide, Methyltriphenylphosphonium iodide, Methyltriphenylphosphonium fluoride, N,N-diethylenethanol ammonium chloride, ethylammonium chloride, Tetramethylammonium chloride, Tetramethylammonium bromide, Tetramethylammonium iodide, Tetramethylammonium fluoride, Tetraethylammonium chloride, Tetraethylammonium bromide, Tetraethylammonium iodide, Tetraethylammonium fluoride, Tetrabutylammonium chloride, Tetrabutylammonium bromide, Tetrabutylammonium iodide, Tetrabutylammonium fluoride, (2-chloroethyl) trimethylammonium chloride, Terbium (Ill) chloride, Zinc (II) chloride, Zinc (II) bromide, Zirconium (Ill) chloride, Iron (Ill) chloride, Tin (II) chloride, Copper (II) chloride, Magnesium (II) chloride; with, one or more other component(s) that can also form a DES, including for example, but without limitation, one or more of the following chemicals chosen from the group: Urea, Formamide, Thiourea, 1-Methylurea, 1,1-Dimethylurea, 1,3-Dimethylurea, Carbohydrazide, Tetramethylurea, 1,3-bis(hydroxymethyl)urea, Benzamide, Girards Reagent T, Lactamide, Acetamide, Fluoroacetamide, Difluoroacetamide, Trifluoroacetamide, Chlorofluoroacetamide, Chlorodifluoroacetamide, Chloroacetamide, Dichloroacetamide, Dichlorofluoroacetamide, Trichloroacetamide, Bromoacetamide, Dibromoacetamide, Tribromoacetamide, Bromofluoroacetamide, Bromodifluoroacetamide, Bromochlorofluoroacetamide, lodoacetamide, Diiodoacetamide, Triiodoacetamide, 2-Methyl-2,2-difluoroacetamide, 2-Methyl-2-fluoroacetamide, 2,2-Dimethyl-2-fluoroacetamide, 2-Ethyl-2,2-difluoroacetamide, 2-Ethyl-2-fluoroacetamide, 2,2-Diethyl-2-fluoroacetamide, 2-Propyl-2,2-difluoroacetamide, 2-Propyl-2-fluoroacetamide, 2,2-Propyl-2-fluoroacetamide, 2-Fluoropropionamide, 3-Fluoropropionamide, 2,2-Difluoropropionamide, 2,3-Difluoropropionamide, 3,3-Difluoropropionamide, 3,3,3-Trifluoropropionamide, 2-Fluoro-3,3,3-trifluoropropionamide, 2-Chloro-3,3,3-trifluoropropionamide, 2,2-Chloro-3,3,3-trifluoropropionamide, 2-bromo-3,3,3-trifluoropropionamide, 2,2-Bromo-3,3,3-trifluoropropionamide, Pentafluoropropionamide, Heptafluorobutyramide, Trimethylacetamide, 1-(Trifluoroacetyl)imidazole, N,O-Bis(trifluoroacetyl)hydroxylamine, Bistrifluoroacetamide, N-Methyl-fluoroacetamide, N-Methyl-difluoroacetamide, N-Methyl-trifluoroacetamide, N-Methyl-chlorofluoroacetamide, N-Methyl-chlorodifluoroacetamide, N-Methyl-chloroacetamide, N-Methyl-dichloroacetamide, D N-Methyl-dichlorofluoroacetamide, N-Methyl-trichloroacetamide, N-Methyl-bromoacetamide, N-Methyl-dibromoacetamide, N-Methyl-tribromoacetamide, N-Methyl-bromofluoroacetamide, N-Methyl-bromodifluoroacetamide, N-Methyl-bromochlorofluoroacetamide, N-Methyl-iodoacetamide, N-Methyl-diiodoacetamide, N-Methyl-triiodoacetamide, N-Methyl-2-methyl-2,2-difluoroacetamide, N-Methyl-2-methyl-2-fluoroacetamide, N-Methyl-2,2-dimethyl-2-fluoroacetamide, N-Methyl-2-ethyl-2,2-difluoroacetamide, N-Methyl-2-ethyl-2-fluoroacetamide, N-Methyl-2,2-diethyl-2-fluoroacetamide, N-Methyl-2-propyl-2,2-difluoroacetamide, N-Methyl-2-propyl-2-fluoroacetamide, N-Methyl-2,2-propyl-2-fluoroacetamide, N-Methyl-2-fluoropropionamide, N-Methyl-3-fluoropropionamide, N-Methyl-2,2-difluoropropionamide, N-Methyl-2,3-difluoropropionamide, N-Methyl-3,3-difluoropropionamide, N-Methyl-3,3,3-trifluoropropionamide, N-Methyl-2-fluoro-3,3,3-trifluoropropionamide, N-Methyl-2-chloro-3,3,3-trifluoropropionamide, N-Methyl-2,2-chloro-3,3,3-trifluoropropionamide, N-Methyl-2-bromo-3,3,3-trifluoropropionamide, N-Methyl-2,2-bromo-3,3,3-trifluoropropionamide, N-Methyl-pentafluoropropionamide, N-Methyl-heptafluorobutyramide, N,N-Dimethyl-2,2,2-trifluoroacetamide, N-Ethyl-2,2,2-trifluoroacetamide, N,N-Diethyl-2,2,2-trifluoroacetamide, N-(Hydroxymethyl)Trifluoroacetamide, Ethyltrifluoroacetate, Dithiothreitol, Dithioerythritol, Beta-mercaptoethanol, Penicillamine, Tiopronin, Acrylamide, Methanol, Ethanol, Propanol, Butanol, Formaldehyde, Glutaraldehyde, Taurine, Aconitic acid, Adipic acid, Benzoic acid, Citric acid, Malonic acid, Malic acid, DL-Maleic acid, Oxalic acid, Phenylacetic acid, Phenylpropionic acid, Succinic acid, Levulinic acid, Tartaric acid, Gallic acid, p-Toluenesulphonic acid, Glycine, Alanine, Valine, Leucine, Isoleucine, Serine, Threonine, Tyrosine, Cysteine, Methionine, Aspartic acid, Asparagine, Glutamic acid, Glutamine, Arginine, Lysine, Histidine, Phenylalanine, Tryptophan, Proline, Ethylene glycol, Triethyleneglycol, Glycerol, Resorcinol, Phenol, 1,2-propanediol, 1,3-propanediol, 1,4-Butanediol, 1,5-Pentanediol, 1,6-Hexanediol, 1,8-Octanediol, 1,12-Dodecanediol, m-Cresol, Imidazole, 1-Methylimidazole, 4-Methylimidazole, N-Methylpyrrolidone, N-Ethylpyrrolidone, N-Benzylpyrrolidone, 2-imidazolindone, tetrahydro-2-pyrimidione, Guanidine, Guanidine HCl, Guanidine isothiocyanate, Guanidine sulphate, Ammonium acetate, Ammonium bicarbonate, Ammonium chloride, Ammonium citrate dibasic, Ammonium formate, Ammonium iodide, Ammonium nitrate, Ammonium phosphate monobasic, Ammonium phosphate dibasic, Ammonium sulfamate, Ammonium sulfate, Ammonium tartrate dibasic, Ammonium isothiocyanate, Ammonium benzoate, Ammonium bromide, Ammonium fluoride, Ammonium hydrogensulphate, Ammonium trifluoroacetate, Ammonium thiosulphate, Adonitol, Ribitol, Rhamnose, Trehalose, D-Sorbitol, L-Sorbitol, Sorbose, Xylitol, Glucose, Sucrose, Lactose, Fructose, Maltose, Mannose, Mannitol, Arabinose, Galactose, Raffinose, Inositol, Erythritol or Xylose.

It should also be noted that there is no particular maximum number of DES components in the DES mixture, for example 2, 3, 4, 5, 6, 7, 8, 9, 10 or more components can be mixed to produce a DES mixture, usually but necessarily, in integer molar ratios for example in a two component DES mixture, component 1 and component 2 can be mixed in the following ratios: 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10 (mol:mol) or for example in a three component DES mixture, component 1, component 2 and component 3 can be mixed in these ratios: 10:1:1, 9:1:1, 8:1:2, 7:1:3, 6:1:4, 5:1:5, 4:1:6, 3:1:7, 2:1:8, 1:1:9, 1:2:8, 1:3:7, 1:4:6, 1:5:5, 1:6:4, 1:7:3, 1:8:2, 1:9:1, 1:10:1 (mol:mol:mol).

An additive may be used in combination with the DES mixture. The purpose of the additive is to improve the properties of the DES mixture, for example, RNA and/or DNA stabilisation, reduction of enzyme inhibition, viral inactivation or another property.

An ‘additive’ is defined here as any substance that can be dissolved in a particular DES mixture, and can be present in amounts greater than, equal to or less than the total amount of the DES mixture (weight:weight). It should also be noted that the additive does not necessarily have any particular effect on the freezing point of the DES mixture or form hydrogen bonds with any of the DES components but endows the DES mixture with a unique property.

The purpose of the additive is to enhance the property of the DES mixture for the particular application, for example RNA stabilisation, storage, transport, and/or cell or tissue fixation. Byway of example only, the additive can be a; (i) colourant or dye to aid in handling or staining or processing the tissue such as H&E stain, Coomasie Blue, Methylene Blue, Xylene cyanol, Crystal violet, fuchsin, Acridine orange, DAPI, Carmine, Eosin, Ethidium bromide, Bismarck brown, Hoechst, Malachite green, Methyl green, Neutral blue, Nile blue, Osmium tetroxide, Rhodamine or Safranin, (ii) detergent, quaternary ammonium salt or saponin to improve penetration of the cell plasma membrane with the DES mixture, such as SDS, sodium lauryl sulphate (SLS), cetyltetrabutylammonium bromide (CTAB), tetrabutylammonium bromide (TBAB), sodium deoxycholate, Brij-35, Brij-58, NP-40, Triton X-100, Triton X-114, Tween-20, Tween-80, Octyl beta glucoside, CHAPS, Solanine, (iii) anti-microbial such as an antibiotic or antiseptic, such as streptomycin or penicillin, sodium nitrate, sodium nitrite or sodium benzoate, (iv) protein precipitant such as Trichloroacetic acid, Ammonium sulphate, Sulphosalicyclic acid, Zinc salt such as ZnC12 or ZnSO4., (v) desiccant to remove excess water from the DES mixture such as Molecular sieves 4 A, silica gel, dry polyacrylamide, dry polyacrylic acid, super adsorbent polymers, CaCl₂ or LiCl, (vi) a probe, a hybridising complementary nucleic acid, a peptide, protein, nucleic acid or labelled molecule, an internal control, a blocking sequence or a carrier nucleic acid such as (a) ss or ds RNA or DNA sequences, (b) a peptide, enzyme or other protein such as an antibody, (c) molecules for detecting an analyte such as a biotin, horseradish peroxidase, avidin, streptavidin, fluorescent labelled molecule such as fluorescein, Texas Red, Alexa Fluor™ labelled LNA, (d) a Molecular Beacon™, a Scorpion Probe™, (vii) anti-oxidant to remove oxygen from the sample and reduce damaging oxidative effects during storage on for example the RNA or DNA nucleobases, such as Vitamin C or glutathionine, (viii) ribonuclease inhibitor such as Rnasin, SUPERase.IN™, RNaseOUT™, an anti-lactoferrin (Rnase) antibody or inhibitor, or a protease inhibitor such as phenylmethylsulfonyl fluoride (PMSF), diisopropyl fluorophosphate (DFP), aprotinin or Pefabloc SC™, (ix) buffer to stabilise the pH between 5-8 or more preferably between 6 and 7, used in the range 0.5-20 mM such as Tris-HCl, PIPES, MES, HEPES, MOPS, MOPSO, CAPS, CAPSO, BIPES, phosphate, imidazole, (x) chelator to remove divalent metal cations, used in the range 0.5-20 mM such as BAPTA, EDTA, EGTA, citric acid, D-Penicillamine, (xi) dissolved oxygen (in the final concentration range of 2-20%), and/or CO₂ (in the final concentration range of 0.01-5%, a non-reactive gas such as Argon (in the final concentration range of 1-20%) and or a buffer, nutrients (such as glucose and amino-acids) to enhance cell, tissue and organism viability, and/or (xii) an alcohol in the range of 1-20% (wt:wt) to reduce DES viscosity such as methanol, ethanol, propanol or tert-butanol.

A non-exhaustive list of possible additives to a DES mixture are: Ammonium p-toluenesulphonic acid, Sodium p-toluenesulphonic acid, Ammonium sulphate, Ammonium chloride, Ammonium thiosulphate, Sodium dodecyl sulphate, Sodium lauryl sulphate, Sodium Benzoate, Dodecyldimethyl(3-sulphopropyl)ammonium hydroxide, Dimethylbenzene sulphonic acid, Congo Red, Giemsa, DAPI, Ethidium bromide, Mallory's stain, Orcein, Aldehyde fuchin, Osmium tetroxide, Chromium trioxide, Chromic acid, Feulgen, Dichromate, Mercuric chloride, Haematoxylin and Eosin stain (H&E), Formaldehyde, Glutaraldehyde, Acetone, Ethanol, Methanol, Methyltriphenylphosphonium bromide, Cetyltetrabutylammonium bromide (CTAB), tetrabutylammonium bromide (TBAB), sodium deoxycholate, Brij-35, Brij-58, NP-40, Triton X-100, Triton X-114, Tween-20, Tween-80, Octyl beta glucoside, CHAPS, Solanine, kanomycin, streptomycin or penicillin, sodium nitrate, sodium nitrite or sodium benzoate, ss or ds RNA or DNA sequences, ss or ds RNA or DNA labelled sequences, an aptamer, a peptide, enzyme, antibody, biotin, biotin labelled molecule, horseradish peroxidase, avidin, streptavidin, GFP or variant thereof, Luciferase, Fluorescein, Rhodamine, Texas Red, Alexa Fluor™, LNA, labelled LNA, a Molecular Beacon™, a Scorpion Probe™, FISH probe, bDNA, PCR primer, oligo (dT), PNA, anti-oxidant, Rnasin, SUPERase.IN™, RNaseOUT™, phenylmethylsulfonyl fluoride (PMSF), diisopropyl fluorophosphate (DFP), aprotinin or Pefabloc SC™, Tris-HCl, PIPES, MES, HEPES, MOPS, MOPSO, CAPS, CAPSO, BIPES, phosphate, imidazole BAPTA, EDTA, EGTA, citric acid, D-Penicillamine, O2, CO2, N2, Argon, propanol, tert-butanol, Trichloroacetic acid, sulphosalicyclic acid, Water, Methanol, Ethanol, Propanol, Butanol, Tetramethyl urea, Imidazole, 1-Methylimidazole, 1-Ethylimidazole, 1-Benzylimidazole, 4-Methylimidazole, N-Methylpyrrolidone, N-Ethylpyrrolidone, N-Benzylpyrrolidone, Guanidine, Guanidine HCl, Guanidine isothiocyanate, Ribitol, Rhamnose, Trehalose, D-Sorbitol, L-Sorbitol, Sorbose, Xylitol, Glucose, Sucrose, Lactose, Fructose, Maltose, Mannose, Mannitol, Arabinose, Galactose, Raffinose, Inositol, Erythritol, Xylose, Zinc acetate, Zinc EDTA, Zinc phosphate, Zinc Trifluoroacetate, Zinc citrate, Zinc PTSA, Zinc gluconate, Zinc chloride and/or Zinc sulphate. Non-dissolvable additives to DES mixtures include but not limited to: Paraffin, Silica gel, Sodium sulphate or Molecular Sieves™, Polyacrylamide, Aerogel, Polyacrylic acid and/or a Quantum Dot.

It should be understood that, for example, viral diagnostics such as the well-known RT-PCR technique that there is either a qualitative or quantitative result obtained. For example, in the case of HIV testing, initially a tested patient is informed that they are either positive or negative for the virus with no specific viral titres reported. If the result is positive the patient will then be commonly put on a therapy which suppresses the virus. It then becomes important to know what the viral titre is, so that the effect of the antiviral therapy can be determined and drug resistance monitored. The initial diagnostic result can be simply a RT-PCR test whilst the latter requires a quantitative test, commonly carried out by RT-qPCR.

It will be understood by one skilled in the art that a qualitative RT-PCR result is less sensitive to the effects of a mild inhibition of the reaction whilst for a quantitative test such as RT-qPCR the effect of inhibition is immediately apparent by an increase in the Ct value (i.e. the amount of inhibition). The present methods may be applied to both qualitative and quantitative tests whether enzymatic or non-enzymatic and including PCR, qPCR, RT-PCR, RT-qPCR or other analytical detection methods such as SHERLOCK, hybridisation tests, bDNA hybridisation tests, and isothermal tests including NASBA, tHDA, RPA, SDA or LAMP assays.

Provided herein are methods for detecting an analyte molecule, commonly a biomolecule such as a single-stranded or double-stranded RNA or DNA molecule in a non-purified cellular, including multicellular sample, or a non-cellular sample such as a viral sample. Sample types include but are not limited to a viral particle including bacteriophages, a prokaryote or eukaryote cell, or a cell infected with one or more viruses.

There are many medically important RNA viruses such as Influenza, HPV, Foot and Mouth Disease Virus, Influenza, SARS including SARS-CoV, SARS-CoV-2, Norwalk, Rotavirus, Poliovirus, Ebola virus, Marburg virus, Lassa virus, Hantavirus, Rabies, Influenza, Yellow fever virus, Corona Virus, SARS, West Nile virus, Hepatitis A, B, C, (HCV) and E virus, Japanese encephalitis virus, Russian tick borne encephalitis Dengue fever virus, toga (e.g. Rubella), Rhabdo (e.g. Rabies and VSV), Picorna (Polio and Rhinovirus), Myxo (e.g. influenza), retro (e.g. HIV, HTLV), bunya, corona and reoviruses which have profound effects on human health including viroid like viruses such as hepatitis D virus and plant RNA viruses and viroids such as Tobus-, Luteo-, Tobamo-, Potex-, Tobra-, Como-, Nepo-, Almo-, Cucumo-, Bromo-, liar-viruses, Coconut cadang-cadang viroid and potato spindle tuberviroid which all have profound effects on agricultural production.

Other pathogens such as fungi and/or bacteria including mycobacterium, a cell carrying one or more viruses integrated in its genome such as HIV or HPV, nucleoli, nuclei, cellular cytoplasm or components of cells such as organelles including mitochondria, golgi, endoplasmic reticulum or cell membrane and vacuoles.

The analyte is chosen from RNA and/or DNA or a covalent mixture of both. Of particular interest are prokaryotic and eukaryotic genomic DNA, mitochondrial DNA, RNA including miRNA, mRNA and lncRNA, but of greatest interest are pathogenic bacteria and those associated with food safety issues, and particularly RNA and DNA viruses with one or more RNA and/or DNA molecules whether single-stranded, double-stranded, positive or negative sense, segmented or not segmented, enveloped or not enveloped which can be either pathogenic or non-pathogenic including adenoviruses, more specifically betacoronaviruses, more specifically those causing SARS and specifically SARS-CoV-2 responsible for the COVID-19 pandemic.

The RNA can be found, derived or associated with a sample such as a virus, cell, circulating tumour cell, cerebrospinal fluid, bronchoalveolar lavage, serum, plasma, blood, exosomes, other microvesicles preserved samples such as FFPE blocks or sections, biopsies, solid or liquid tissues or other biological samples. It can also be an oligomer of deoxy- and ribonucleotides, deoxy- or ribo-oligonucleotides, plasmid DNA, genomic DNA, mitochondrial DNA, RNA such as microRNA (miRNA), piRNA, siRNA, tRNA, viriods, circulating RNA, circular ss or ds non-coding RNA, hnRNA, mRNA, rRNA such as the 5S, 5.8S, 16S, 18S, 23S and 28S rRNA species, and viral RNA derived from for example HIV, HCV, West Nile Disease Virus, Foot and Mouth Disease Virus, Influenza, SARS, or HIV RNA, and/or extracellular RNA (exRNA).

RNA analysis methods that would benefit from the present methods include in vitro or in vivo protein translation of non-purified mRNA templates, RNA dependent RNA polymerisation, DNA dependent RNA polymerisation, RNA:protein interaction studies, RNA electrophoresis and sedimentation, molecular controls and standards, RNA bioconjugates, RNA ligation, RNA sequencing, reverse transcription (RT), PCR, qPCR, RT-PCR, RT-qPCR, bDNA, and microarrays including the preparation of probes, fluorescent nucleic acid labelling, NASBA, tHDA, RPA, SDA or LAMP assays, RNAi, miRNA techniques such as extraction and quantification and those methods requiring quality control and/or quantitative or qualitative measurements of RNA.

An Rnase inactivation step can be carried out to sufficiently inactivate Rnase and allow subsequent stabilisation with a DES mixture without a significant degradation of the RNA. Numerous methods are known in the art for inactivating Rnases and/or overcoming inhibitors of reverse transcriptase such as enzymatic degradation with proteases such as trypsin, chymotrypsin, papain or proteinase K, reduction of the disulphide bond with β-mercaptoethanol (Chirgwin, et al., (1979) Biochemistry, 18:5294-5299), dithiothreitol, dithioerythritol, glutathione or TCEP, BSA, spermine, heparinase, addition of Rnasin protein (Life Technologies, USA), RNAsecure™ (Life Technologies, USA), treatment with a chaotrope such as guanidine HCl, guanidine thiocyanate (Chomczynski and Sacchi, Anal. Biochem., 162:156-159, 1987; Sambrook, et al., “Molecular Cloning, A Laboratory Manual,” pp. 7.16-7.52, 1989.), urea, formamide, formaldehyde or sodium iodoacetate, treatment with a detergent such as Tween-20, Tween-40, Tween-60, Tween-80, Digitonin, NP-40, Nonidet P-40, Triton X-100, Brij-35, Igepal-CA630, Saponin, Tergitol, SLS, or SDS, EDTA, EGTA, sodium citrate, heat or acid denaturation, inhibition with vanadyl ribonucleoside complexes (Berger and Birkenmeier, 1979) or cross-linking with glutaraldehyde. Residual Rnase activity prior to treatment can be monitored using an RnaseAlert™ kit (Life Technologies, USA). Methods for inactivating Rnases in tissues are set out in U.S. Pat. No. 6,777,210.

The present methods particularly concern viruses that can be found in the context of a sample including a clinical or animal sample such as saliva, sputum, a nasal, nasopharanyngeal or oronasopharyngeal sample, phlegm, a sample from the tongue, teeth, nose, blood, sweat, tears, plasma, serum, PBMCs, CSF, urine, semen, mucus, nasal mucus, milk, amniotic fluid, a cell or part of a cell, groups of cells, a tissue, an organ, an environmental sample such as an airborne droplet derived from a sneeze, a cough or expired breath, a contaminated surface such as a face mask or glove, medical waste, faecal matter, sewage, sea or fresh water, a filtered sample, a stored or fresh sample, and/or a sample that has been treated with a reagent such as a fixative and/or viral inactivator.

The sample can be pre-treated prior to analysis with one or more enzymes such as proteases, nucleases, lipases and amylases in order to; (i) reduce its viscosity (liquefy) and therefore improve the handling properties such as pipetting, (ii) improve the sensitivity of detection of the analyte by breaking down the sample matrix surrounding the analyte nucleic acid such as sputum mucin, the bacterial and/or eukaryotic cell membrane, cell wall and/or cytoplasm, the viral envelope and/or capsid, nucleoproteins bound to the analyte nucleic acid.

Examples of suitable enzymes include (i) proteases such as pronase, collagenase, liberase, trypsin, papain, (ii) nucleases to reduce the viscosity of the sample but without destroying the analyte such as Dnase I, benzonase, ribonuclease. A DNA nuclease would be preferable to use when analysing an RNA analyte and vice-versa. In the case of proteases and nucleases, their activity should typically be abrogated prior to sample addition to the analytical test to avoid inhibition of the test.

Other pre-treatments can include mucolytic agents such as amylases, proteases, reducing agents such as DTT, DTE, beta-mercaptoethanol, carbocisteine, acetylcysteine and/or TCEP, erdosteine, detergents preferably non-ionic detergents that are less likely to interfere with the such as Igepal-CA630, Triton X-100 and/or Tween-20. Mixing the sample with small quantity, e.g. an amount in the range 2.5 to 3.5% by volume, of a deep eutectic solvent such as N,N,N-trimethylglycine:trifluoroacetamide may reduce the viscosity of the sample and allow for easier handling of the sample.

The samples used in the practice of the present methods are not substantially purified. Pre-treatment does not refer to RNA or DNA samples that have been substantially purified to remove non-nucleic acid components. Substantial purification refers to nucleic acid samples with an OD 260/280 nm purity ratio of 1.8 or more.

A non-purified crude cellular sample of RNA may however be mixed with a control nucleic acid sample. The control nucleic acid sample may have an OD 260/280 nm purity ratio of 1.8 or more. The resulting mixture may have an OD 260/280 nm ratio of less than 1.8.

Partially purified nucleic acid samples may be used, for example those that have undergone an initial step of removal of non-nucleic acid material from the nucleic acid analyte, for example by removal of non-viral and/or non-cellular material from the virus or cell such as by removing sputum from the virus and/or cell. Partial purification refers to nucleic acid samples with an OD 260/280 nm purity ratio of less than 1.8. Determining the OD 260/280 nm purity ratio using for example a spectrophotometer is a well-known and generally recognised measure of nucleic acid purity.

Partial purification may, in certain examples, involve a concentration step of the desired virus and/or cell in the sample by for example membrane or bed filtration, chromatography including size exclusion, ion exchange and affinity chromatography, electrophoresis, centrifugation and/or pelleting, immobilisation on a charged, hydrophobic or affinity membrane, filter, bead or particle including magnetic beads and particles priorto the analysis. Affinity concentration may be for example by means of a free or bound antibody and/or other biomolecule, such as heparin sulphate, heparan sulphate, a cationic material, a polysaccharide, a glycoprotein, lectin, a virus receptor protein such as the ACE, ACE2 or CD4 cell surface protein.

A virus may be concentrated by binding to individual cells or to a solid phase, e.g. beads or particles. The cells or solid phase may subsequently be used as carriers to transport the virus prior to and/or during an analytical procedure.

The solid-phase bearing the surface treatment used to concentrate or capture the virus, cell or even non-purified cfRNA and cfDNA analyte can be for example, a bead, particle, cell, probe, dipstick, paper, filter, membrane, needle, centrifuge tube or multiwell plate. The solid phase may consist of an organic or inorganic particle, a polymeric linear, globular or cross-linked molecule or resin. It may be made of a variety of materials or material composites such as acrylamide, agarose, cellulose, polyamide, polycarbonate, polystyrene, polyethylene, polypropylene polytetrafluoroethylene, nitrocellulose, latex, aluminium, copper, nickel, iron, a metal oxide, a mixture of metals such as an iron-zinc blend or an oxide thereof, lithium iron III oxide, glass, hydroxylapatite or silicon.

The carrier can alternatively be a fixed or a living cell including a cell such as a tissue culture cell genetically modified or naturally expressing or preferably over-expressing a virus binding partner such as the animal or human ACE2 protein (Mossel et a., (2005) J Virol. 79(6): 3846-3850) on its surface as a means to capture one or more of a coronavirus in particular a betacoronaviruses and specifically the SARS-CoV-2 virus. Following such virus capture on the cell surface by means of a biomolecule that has an enhanced affinity for a virus such as the ACE2 protein, the cell and virus can then be consequently treated together with a reagent such as a covalent fixative including formaldehyde, or a non-covalent fixative such as a DES, an alcohol including methanol and ethanol, a metal salt, an acid or a mixture of any of these in order to enhance and stabilise the attachment of the virus to the cell surface so that the virus and cell can be handled as a single particle.

The cell, bead or particle bearing the immobilised virus on its surface can then be used with or without a prior nucleic acid purification step, for use with an assay such as PCR, qPCR, RT-PCR, RT-qPCR, LAMP, SHERLOCK, a hybridisation assay including those using bDNA, single cell DNA-seq or single cell RNA-seq including Rhapsody (Becton Dickerson), Chromium (10×Genomics), ddSEQ (Bio-Rad), SMART-seq-2, drop-seq, SeqWell, ATAC-seq, MARS-seq, QUARTZ-seq2 in order to detect the presence or absence of the virus.

Specifically for single cell DNA-seq and single cell RNA-seq, the presence of a cell is generally considered essential in order to transport the analyte DNA and/or RNA to the reaction vessel or droplet as a discrete localised population rather than being dispersed throughout the sample. Here we envisage not only the use of cells for such so called ‘single cell’ techniques but also beads and particles capable of associating with a biological sample such as a virus, bacteria or eukaryotic cell including pathogens and parasites. We describe a series of different surface modified beads that can collect and bind to specific types of biological targets, for example a surface modification such as heparin sulphate that has the general property of binding viruses, or, alternatively, an ACE2 protein surface that can bind to coronaviruses such as SARS-CoV-2. Other types of useful beads are protein A beads (Sigma-Aldrich Cat. No. LSKMAGA02) that have the property of binding to IgG antibodies making them very useful as general particles with specificity brought by the use of particular types of antibodies such as an anti-SARS-CoV-2 spike protein antibody. Other useful beads are streptavidin coated beads (Sigma-Aldrich Cat. No. LSKMAGT) for binding to biotin conjugated biomolecules such as antibodies, oligonucleotides, oligoribonucleotides, LNA, PNA and other chemically modified oligonucleotides, polysaccharides and proteins.

These surface modified beads or particles are preferably sufficiently small as to be able to be passed through a microfluidic canal, encapsulated in a droplet or enter the nanowell that are commonly used for single cell analysis. Useful sizes are less than 50 μm diameter, preferably less than 40 μm.

The surface modified beads or particles preferably have enough surface area as to be able to bind to and attach to the particle containing the analyte (DNA, RNA or protein) whether this be free DNA and/or DNA, non-purified RNA and/or DNA a virus, a bacteria, a yeast, a parasite or a eukaryotic cell or cells. The attachment should also be sufficiently strong that the analyte is not lost during processing and compartmentalisation of the bead or particle during the analytical steps but is sufficiently free to serve as an appropriate template for example during reverse transcription, RT-PCR or PCR.

The attachment of the analyte to the surface-modified beads or particles may be strengthened by for example fixing the particle to the cell, bead or particle with a cross-linking agent such as an aldehyde including formaldehyde and glutaldehyde, a photoreactive cross-linker, a bifunctional N-hydroxysuccinimide, NHS-linker, a reversible disulphide bond linker, prior to analysis. Cross-linking and conjugation methods are well known and a useful guide is: https://www.thermofisher.com/fr/fr/home/life-science/protein-biology/protein-labeling-crosslinking/protein-crosslinking/crosslinker-application-reference-guide.html

Cells to be analysed can be scraped or obtained from a healthy or a viral and/or bacterial infected area of a patient's body such as the skin, mouth, eye, lips, tongue, throat, nose, trachea, oesophagus, intestine including the colon or rectum, or a fine needle aspirate (FNA) biopsy or other biopsy from the patient, by means of a swab, probe, baton, pipette, scraper, needle, syringe, aspirate and then used for an analytical test, in particular this would be very useful as a source of viruses or bacteria that are on the surface of, or inside the sampled cell to test a patient's infection status. Methods for preparing suitable single cells for analysis using DES are set out in detail in Goldsborough and Bates; PCT/EP2019/069846 (published as WO 2020/020913 A1).

As there is insufficient RNA in a single virus to reproducibly detect it using techniques currently available, 1-500 viable individual single tissue culture cells expressing or over-expressing large amounts (e.g. approximately 100, 1000, 10,000 or 100,000) of the SARS-CoV-2 binding receptor ACE2 on the cell surface (Mossel et al., (2005) J Virol. 79(6): 3846-3850) allowing one or more SARS-CoV-2 viruses to bind and become immobilised to the surface of a single cell. Batches of 10, 50, 100, 200, 500 or more ACE2 expressing cells are barcoded with a unique sequence allowing the patient identity to be linked to a specific population of cells.

Cell barcoding can be carried out in a variety of ways: (i) by a unique sequence feature of the cell such as a genetic variant, including a variant mRNA sequence not found in other population of cells, examples include; (a) mouse cells can easily be identified in a population of human cells by way of its mRNA sequence variants, (b) a human tissue culture cell such as HeLa that can easily be identified from other human tissue culture cells such as HEK 293 cells by way of each unique collection of mRNA that is specific to the HeLa or HEK 293 cell, (c) genetically engineered cells that have specific sequences introduced into one or more genes so that the mRNA have a unique expressed sequence tag, such sequences can be introduced by CRISPR-Cas9 methods, (d) with B-cells or T-cells expressing unique TCR or antibody sequences unique to that cell, (e) ideally the cells are identical except for one or more small sequence differences in their genomes and/or mRNA transcripts so that they can be unequivocally detected and identified by means of scRNA-seq or scDNA-seq. All the cells have the common feature of expressing a virus docking protein such as ACE2 or CD4. Alternatively, genetically identical cells can be uniquely barcoded with a unique sequence by; (a) stable or transient transfection with an oligonucleotide, or plasmid bearing a gene that can be expressed in the cell allowing identification, (b) binding to an antibody-oligonucleotide conjugate ‘hashtag’ specific for a ubiquitously expressed marker protein such as CD298 or beta2-Microglobulin allowing unique cell population identification (TotalSeq, Biolegend Inc, Cat. No. 394601, 394603, 394605) is well known (Stoeckius (2018) Genome Biol; 19: 224).

It is preferred that an alternative reverse transcriptase primer is used than oligo(dT) for several reasons; (i) not all RNA viruses have a poly(A) tail and therefore they would not be copied into cDNA, and (ii) cDNA synthesis and sequencing of the mRNA content of the cell (which is of no interest other than potentially identifying the cell and therefore the identity of the patient) carrying ACE2 as well as the viral genome would lead to reagent wastage, excess sequencing cost and time.

Targeted virus capture with a docking protein such as ACE2 can potentially capture not only SARS-CoV-2 but also SARS-CoV, if it is desired that detection of both viruses is carried out it will be understood that a reverse transcriptase primer is used that can hybridise to both viral genomes. In the case of the polyadenylated SARS-CoV-2 and SARS-CoV RNA genomes this could either be an oligo(dT) containing primer, but also a specific SARS virus primer with sufficient degeneracy to hybridise to both viruses. It will be evident to one skilled in the art that depending on the choice or choices of 1 or more docking protein (e.g. ACE and ACE2) and/or the specificity of the reverse transcriptase primer it will be possible to target either 1 specific type of virus (e.g. SARS-CoV-2) and 1 specific strain, or if preferred, variants such as strains (e.g. SARS-Cov-2 S) and subtypes, as well as related species of viruses (SARS-CoV) or whole groups of viruses such as the betacoronavirus family. It will also be evident that it will be possible to target multiple types of viruses by combining multiple different docking proteins such as ACE2 for SARS-CoV-2 and CD4 for HIV. There is no particular limitation for the number of viral docking proteins that can be displayed on a cell surface and it will be understood that determining the natural level of cell surface expression or over-expressing a recombinant cell surface protein are well known methods.

It is also possible to capture viruses using non-specific means, for example by using cells producing sialic acid glycoproteins on their surfaces to capture influenza A, B, C and D.

When the virus is a single-stranded or double-stranded DNA virus a similar strategy of using an expressed recombinant or naturally expressed cell surface protein. Alternatively, a non-specific cell surface target can also be taken except in this case it is necessary to use a suitable scDNA-seq approach such as Chromium Single Cell CNV Solution (10× Genomics). When the virus does not have a poly(A) tail then a virus specific primer should be used, this reverse transcriptase primer has a sequence complimentary to the viral genome sequence, a unique molecular identifier (UMI), a 10× barcode and sequence R1, and conjugated to the gel-bead in emulsion (GEM) bead (10× Genomics).

Alternatively, the virus specific reverse transcriptase primer can be added to the oligo(dT) solution of the Chromium™ Single Cell V(D)J Solution at a final concentration of 1-10 uM, the viral sequences are then identified from the generated 5′ cDNA library (10× Genomics).

As an alternative to using a living or a fixed cell as the target for virus attachment and transport, it is possible to use instead an organic or inorganic particle, preferably of a size similar to a cell, for example less than 50 μm, preferably less than 40 μm, for example 4-30 μm in diameter. In this case the particle has a surface coating such as glycoproteins, heparin and/or sialic acid as a non-specific target for virus binding, or an antibody specific for a particular virus surface protein such as gp120 or the SARS-CoV-2 spike protein. The bead is used in batches, for example 50 beads per patient clinical sample, multiplexed according to the number of samples to be tested.

The method of protein attachment to the bead can be via a protein A-antibody bridge (e.g. Sigma-Aldrich Cat. No. LSKMAGA02) to the antibody of interest. Alternatively, it can be via a streptavidin-biotin bridge, with the biotin conjugated to the antibody of interest. Yet one more possibility is to cross-link using an aldehyde the antibody to the bead. Such methods to immobilise antibodies onto a bead or other particles are well known in the literature and described elsewhere in this application.

In a clinical setting, it may be essential to be able to determine the identity of the patient from the final sequencing results. This is done by conjugating a unique oligonucleotide ‘tag’ to either the antibody as in CITE-seq (Stoeckius et a., (2017) Nat Methods. 14(9):865-868), or as a biotinylated oligonucleotide attached to a streptavidin coated bead. Conveniently, the streptavidin can serve to both attach the biotinylated oligonucleotide as well as the biotinylated antibody.

The beads and particles provided herein may therefore include: (i) an oligonucleotide with a unique and specific sequence allowing unambiguous identification of the patient identity (suitable oligonucleotide design is amply described in Stoeckius et al., (2017) Nat Methods. 14(9):865-868) and are composed of a PCR handle, a poly(A) tail, a UMI code, a barcode providing identification of the patient, and a mean to be attached to a bead and a cell, (ii) an antibody capable of binding and capturing a specific virus or a group of related viruses, or all viruses in the sample by way of a surface negative charge, and (iii) a solid phase bead that serves as a carrier for both the oligonucleotide and the antibody and provides; (a) simplified handling when they are added to the patient sputum or swab sample, (b) a physical connection between the virus, antibody and oligonucleotide so all can be encapsulated into a droplet emulsion together prior to loading on the scRNA-seq or scDNA-seq platform (e.g. 10× Chromium device or BD Rhapsody) with a bead. Following sequencing the data can be analysed to determine which particles are associated with a viral sequence and how much viral sequence in a qualitative manner, and byway of the oligonucleotide the patient identity. This disclosure therefore provides a means to massively multiplex patient samples for viral testing.

One significant advantage of using a particle such as a bead compared with a cell is that in principle, only the viral genome is analysed and sequenced rather than the entire transcriptome of the carrier cell. This allows for reduced sequencing costs as low sequencing depth only is sufficient to determine the presence or absence of virus and the presence of the oligonucleotide.

### EXAMPLES

### Example 1. Preparing Non-Aqueous Dilutions of Stoichiometric Molar Ratios of Betaine and Trifluoroacetamide

A quaternary ammonium compound and a halogenated organic compound such as a fluorinated organic compound, preferably Betaine and Trifluoroacetamide are mixed in a 1:2 mol:mol ratio and then heated at 70° C. for 30 minutes with shaking until a liquid Deep Eutectic Solvent is obtained.

The DES is then diluted by mixing the DES with a diluent to obtain a composition comprising the quaternary ammonium compound at a concentration in the range 1 mM to 8 M, more preferably 10 to 600 mM, more preferably 40 to 500 mM and most preferably 400 mM or less; and the halogenated organic compound at a concentration in the range 1 mM to 8 M, more preferably 20 mM to 1.2 M, more preferably 80 mM to 1 M and most preferably 850 mM or less.

The diluent is one or more of: a Glycol such as Glycerol, Ethylene glycol, Diethylene glycol, Triethylene glycol, Diethylene glycol monoethyl ether acetate, Polyethylene glycol methyl ether, a low viscosity, low toxicity, room-temperature liquid such as Polyethylene glycols with a MW of 150 to 400,000 Daltons, preferably 150 to 600 Daltons and most preferably PEG 200, PEG 300, PEG 400, PEG 500 and PEG 600, Tetraethylene Glycol, Tetraethylene glycol monomethyl ether, Tetraethylene glycol dimethyl ether, Tetraethylene glycol monoethyl ether, Tetraethylene glycol diethyl ether, Diethyleneglycol monoethyl ether acetate, Tetrahydrofurfuryl alcohol polyethyleneglycol ether, polyethylene glycol methyl ether, polypropylene glycol, polypropylene glycol-polyethylene glycol co-polymers, or another non-aqueous solvent such as DMSO, DMF, Acetone, Dihydroxyacetone, Formamide, Dimethylformamide, 1,3-Diamino-acetone, Formaldehyde, Glutaraldehyde, an alcohol including Pentanol, Butanol including tert-butanol, Propanol, Ethanol, Methanol, 1,2-propanediol, 1,3-propanediol, 1,4-Butanediol, 1,5-Pentanediol, 1,6-Hexanediol, 1,8-Octanediol, 1,12-Dodecanediol, Cresol, Tetramethyl urea, Imidazole, 1-Methylimidazole, 1-Ethylimidazole, 1-Benzylimidazole, 4-Methylimidazole, N-Methylpyrrolidone, N-Ethylpyrrolidone, N-Benzylpyrrolidone, Beta-mercaptoethanol, Ethyl carbonate, Phenol, an oil, a wax, liquid Paraffin, a DES not composed of Betaine or Trifluoroacetamide, Carboxylic acid such as Formic, Acetic, Propanoic, Butanoic, Lactic, Polyacrylic acid, Citric acids and halogenated derivatives thereof, polyvinyl pyrrolidone, polyvinyl alcohol, polyethylene oxide, hydroxypropylcellulose, polyvinyl pyrrolidone-vinyl acetate, N-(2-hydroxypropyl) methacrylamide, Poly(2-alkyl-2-oxazolines), polyphosphoesters and/or poly-ethyleneimine.

Where the Betaine and Trifluoroacetamide is dissolved in more than one non-aqueous solvent, the solvents can be mixed together either before, during or after addition of Betaine and Trifluoroacetamide to form a homogenous liquid.

It is preferred that no solids remain in the final liquid mixture. It is straightforward to test the solubility of the DESs by adding the DES to the solvent, mixing, and observing the amount, if any, of non-dissolved solids.

Molar ratios of Betaine and Trifluoroacetamide that do not form a room-temperature DES liquid such as 1:4, 1:5, 1:6, 2:1, 3:1, 4:1 mol:mol may nevertheless dissolve in non-aqueous solvents such as PEG 200. The use of stoichiometric molar ratios of DES components such as Betaine and Trifluoacetamide that do not form a room-temperature liquid, but that can nevertheless be dissolved in a non-aqueous solvent is contemplated.

### Example 2. Preparing Aqueous Dilutions of Stoichiometric Molar Ratios of Betaine and Trifluoroacetamide

Example 1 is repeated, with the non-aqueous diluent being replaced by an aqueous diluent.

In addition to water, the aqueous diluent may include any of a very large number of aqueous solutions can also be used such as those with dissolved buffers, detergents, salts, proteins, nucleic acids, stabilisers, preservatives, or an aqueous buffer and/or chelator such as Tris-HCl, PIPES, MES, HEPES, MOPS, MOPSO, CAPS, CAPSO, BIPES, phosphate, imidazole, BAPTA, EDTA, and EGTA. The resulting composition may have a pH in the range 6 to 8, most preferably 6 to 7.

### Example 3. Preparing Non-Aqueous Dilutions of Non-Stoichiometric Molar Ratios of Betaine and Trifluoroacetamide

A quaternary ammonium compound and a halogenated organic compound such as a fluorinated organic compound, preferably Betaine and Trifluoroacetamide, in amounts independent of the other component, are each dissolved in one or more non-aqueous solvents such as those set out in Example 1 to give a final Betaine concentration in the range of 1 mM to 8 M, more preferably 10 to 600 mM, more preferably 40 to 500 mM and most preferably 400 mM; and a Trifluoroacetamide concentration in the range of 1 mM to 8 M, more preferably 20 mM to 1.2 M, more preferably 80 mM to 1 M and most preferably 850 mM. The Betaine and Trifluoroacetamide components may be pre-mixed together before, during or after dissolving in the non-aqueous solvent(s). One particularly preferred class of non-aqueous solvents are the Polyethylene glycols, e.g. PEG 200.

### Example 4. Preparing Aqueous Dilutions of Non-Stoichiometric Molar Ratios of Betaine and Trifluoroacetamide

A quaternary ammonium compound and a halogenated organic compound such as a fluorinated organic compound, preferably Betaine and Trifluoroacetamide, in amounts independent of the other component, are each dissolved in an aqueous solvent such as those set out in Example 2 to give a final Betaine concentration in the range of 1 mM to 8 M, more preferably 10 to 600 mM, more preferably 40 to 500 mM and most preferably 400 mM, and Trifluoroacetamide in the range of 1 mM to 8 M, more preferably 20 mM to 1.2 M, more preferably 80 mM to 1 M and most preferably 850 mM. The Betaine and Trifluoroacetamide components may be mixed together before, during or after dissolving in the aqueous solvent(s). One particularly preferred aqueous solvent is water.

### Example 5. Inactivating Viruses Including SARS-CoV-2 Using a Non-Aqueous Dilution of Betaine and Trifluoroacetamide

100 μl of an aqueous sample containing 5% (vol/vol) foetal calf serum and with a titer of 10 log 6 SARS-CoV-2 viral particles is mixed by gentle pipetting with 300 μl of a non-aqueous (PEG 200) solution of a quaternary ammonium compound and a halogenated organic compound such as a fluorinated organic compound, 1 mM to 8 M, more preferably 10 to 600 mM, more preferably 40 to 500 mM and most preferably 400 mM or less of a quaternary ammonium compound and, 1 mM to 8 M, more preferably 20 mM to 1.2 M, more preferably 80 mM to 1 M and most preferably 850 mM or less of a halogenated organic compound, then incubated at 20° C. for either 10 minutes or 30 minutes to inactivate the virus.

The 400 μl mixture is then subjected to a purification step to remove any cytotoxic effect of the inactivation components. The purified samples are then immediately titrated on Vero E6 cells and cultured to establish virus titer by plaque assay compared with a PBS treated controls. Detailed protocols are set out in; Welch, S R., et al (2020) J. Clin. Microbiology, Vol 58:e017113-20.

Alternative stoichiometric molar amounts of Betaine and Trifluoroacetamide are set out in Example 1 and non-stoichiometric molar amounts in Example 3, alternative non-aqueous solvents are also set out in Example 1. It is found that a final Betaine concentration in the range of 10 to 600 mM and a final Trifluoroacetamide concentration in the range 20 mM to 1.2 M is effective for inactivating SARS-CoV-2.

One example composition comprises about 400 mM Betaine and about 850 mM Trifluoroacetamide (final concentrations) in PEG 200. Preferred non-aqueous solvents include PEG in the molecular weight range of 150-600.

### Example 6. Inactivating Viruses Including SARS-CoV-2 Using an Aqueous Dilution of Betaine and Trifluoroacetamide

An identical procedure to that of Example 5 is undertaken, using a quaternary ammonium compound and a halogenated organic compound such as a fluorinated organic compound, preferably using Betaine and Trifluoroacetamide concentrations as set-out in Example 5, except that the non-aqueous solvent is replaced with water or an aqueous buffer such as those set out in Example 2.

It is found that similar aqueous concentrations of Betaine and Trifluoroacetamide inactivated SARS-CoV-2 as non-aqueous concentrations. However only the non-aqueous mixtures inactivate the virus and stabilised the RNA; aqueous mixtures although they inactivate the virus, led to RNA degradation within 3 days at 20° C. as determined by RT-qPCR of the SARS-CoV-2 genome.

### Example 7. Disinfecting Non-Biological Surfaces Using a Non-Aqueous Dilution of Betaine and Trifluoroacetamide

10 ml of a non-aqueous solution of 10% (vol/vol) Betaine:Trifluoroacetamide (1:2 mol:mol) and 90% PEG 200 is dispensed onto the surface to be decontaminated such as a worksurface, medical equipment, or personal protective equipment (“PPE”). A soft paper cloth is used to spread the solution across the entire surface and then left for 30 minutes to allow viral and microbial inactivation. Excess disinfectant is then removed using a paper cloth and rinsed once with water to remove any remaining liquid.

It is found that the decontamination solution did not dry out or evaporate allowing prolonged contact with the surface. Various useful stoichiometric molar amounts of Betaine and Trifluoroacetamide are set out in Example 1 whilst alternatively, non-stoichiometric molar amounts such as 400 mM Betaine and 850 mM Trifluoroacetamide (final concentrations) are set out in Example 3.

### Example 8. Disinfecting Non-Biological Surfaces Using an Aqueous Dilution of Betaine and Trifluoroacetamide

10 ml of an aqueous solution of 10% (vol/vol) Betaine:Trifluoroacetamide (1:2 mol:mol) and 90% water is dispensed onto the surface to be decontaminated such as a worksurface, medical equipment, personal protective equipment (“PPE”). A soft paper cloth is used to spread the solution across the entire surface and then left for 30 minutes to allow viral and microbial inactivation. Excess disinfectant is then removed using a paper cloth and rinsed once with water to remove any remaining liquid.

Various useful stoichiometric molar amounts of Betaine and Trifluoroacetamide are set out in Example 2 whilst alternatively, non-stoichiometric molar amounts are set out in Example 4.

### Example 9. Use of a Non-Aqueous Dilution of Betaine and Trifluoroacetamide as an Antiseptic

1 ml of a non-aqueous solution of 10% (vol/vol) Betaine:Trifluoroacetamide (1:2 mol:mol) and 90% PEG 200 is dispensed onto an infected area of skin and then left for 30 minutes to allow viral and microbial inactivation. Excess antiseptic is then removed using a paper cloth and rinsed once with water to remove any remaining liquid.

Various useful stoichiometric molar amounts of Betaine and Trifluoroacetamide are set out in Example 1 whilst alternatively are set out in Example 3.

### Example 10. Use of an Aqueous Dilution of Betaine and Trifluoroacetamide as an Antiseptic

1 ml of an aqueous solution of 10% (vol/vol) Betaine:Trifluoroacetamide (1:2 mol:mol) and 90% water is dispensed onto an infected area of skin and then left for 30 minutes to allow viral and microbial inactivation. Excess disinfectant is then removed using a paper cloth and rinsed once with water to remove any remaining liquid.

Various useful stoichiometric molar amounts of Betaine and Trifluoroacetamide are set out in Example 2 whilst alternatively, non-stoichiometric molar amounts are set-out in Example 4.

### Example 11. Use as an Inactivating Reagent for Direct to cDNA Applications Including RT-PCR and RT-LAMP

A nasopharyngeal swab or 100 μl saliva specimen is mixed with 200 to 600 μl of a non-aqueous or an aqueous mixture of Betaine and Trifluoroacetamide at concentrations that are set out in Examples 1-4, then incubated at 20° C. for either 10 or 30 minutes to inactivate any pathogens in the sample. Then 1, 2, 5 or 10 μl portions are removed and added directly, without RNA purification, into a 50 μl cDNA reaction as set-out in the manufacturer's instructions (Applied Biosystems TaqPath COVID-19 CE-IVD RT-PCR Kit (Cat. No. A48067) with further information about RT-qPCR kits available (IGI Testing Consortium., Amen, A. M., et al. (2020). Nat Biotechnol 38:791-797. Applied Biosystems. TaaPath COVID-19 Combo Kit Instructions for Use MAN0019181. Thermo Fisher Scientific. TaqPath COVID-19 Multiplex Diagnostic Solution. https://www.thermofisher.com/us/en/home/clinical/clinical-genomics/pathogen-detection-solutions/coronavirus-2019-ncov/genetic-analysis/taqpath-rt-pcr-covid-19-kit.html (2020).

Alternatively 1, 2, 5 or 10 μl portions of the inactivated sample are added to a one- or two-step RT-LAMP reaction as described in Park G S et al. (2020) (J Mol Diagn 22:729-735), Klein et al., medRxiv 2020.07.08.20147561, Ganguli A et al., medRxiv 2020.11.16.20232678. PEG 200 to PEG 600 are preferred non-aqueous solvents whilst water is the preferred aqueous solvent, however other suitable solvents are set out in Examples 1 and 2.

### Example 12. Use as an Inactivating Reagent for Lateral Flow Tests

A nasopharyngeal swab or 100 μl saliva specimen is mixed with 200 to 600 μl of a non-aqueous or an aqueous mixture of Betaine and Trifluoroacetamide at concentrations set-out in Examples 1-4, then incubated at 20° C. for either 10 or 30 minutes to inactivate any pathogens in the sample. Then 1, 2, 5 or 10 μl portions are removed and added separately to the loading port of the lateral flow test as described (Conklin S E et al., medRxiv 2020.07.31.20166041, Rudolf F et al., medRxiv 2020.08.18.20177204). PEG 200-PEG 600 are preferred non-aqueous solvents whilst water is the preferred aqueous solvent, however other suitable solvents are set-out in Examples 1 and 2.

A variation of this example is as follows. A nasopharyngeal swab or 100 μl saliva specimen was mixed with 200-600 μl of a non-aqueous or an aqueous mixture of Betaine and Trifluoroacetamide at concentrations set out in Examples 1 to 4, then incubated at 20° C. for either 10 or 30 minutes to inactivate any pathogens in the sample. Then either 1, 2, 5, 10 μl portions were removed, added and mixed with 330 μl of sample extraction buffer included with the kit (SARS-CoV-2 Rapid Antigen Test Nasal, Ref: Roche, Germany 9901-NCOV-03G) and 3-4 drops added to the loading port of the lateral flow test as set out in the manufacturer's instructions, or as described (Conklin S E et al., medRxiv 2020.07.31.20166041, Rudolf F et al., medRxiv 2020.08.18.20177204). PEG 200-PEG 600 are preferred non-aqueous solvents whilst water is the preferred aqueous solvent, however other suitable solvents are set out in Examples 1 and 2.

It was found that 10% Betaine:Trifluoroacetamide (1:2 mol:mol) with 90% of either PEG 200, Tetraethylene glycol or Tetraethylene glycol monomethyl ether is compatible with the SARS-CoV-2 Rapid Antigen Test or alternatively a SARS-CoV-2 IgG/IgM Rapid Test (Ref: Acon, China L031-11711) up to a final concentration in the loaded sample of 30%. For both the Antigenic and Serology test, both the Control line and detector line (presence of Ag or IgG/IgM) were positive when used with the positive Ag control swab (Ref: Roche, Germany 9901-NCOV-03G) or blood from a post-covid sero-converted patient.

### Example 13. Inactivating virus in a saliva sample

To 3 ml of an aqueous or non-aqueous solution of a quaternary ammonium compound and a halogenated organic compound such as a fluorinated organic compound mixture, in a 10 ml tube is added 1 ml of saliva from a potentially SARS-CoV-2 infected patient that is required to be tested. The mixture is gently shaken and then incubated in the quaternary ammonium compound and halogenated organic compound solution for 60 minutes at room temperature to allow virus inactivation to occur prior to carrying out an analytical test for the SARS-CoV-2 or other virus, with or without analyte purification, such as LAMP, RT-LAMP, LamPORE test, DNAnudge test, Optigene RT-LAMP, PCR, RT-PCR, geneXpert RT-PCR or BioFire FilmArray RT-PCR or antigen testing such lateral flow.

Alternatively, the saliva sample may be transported and stored in a solution of a quaternary ammonium compound and a halogenated organic compound such as a fluorinated organic compound until needed for an analytical test. A non-aqueous solution of the quaternary ammonium compound and a halogenated organic compound such as a fluorinated organic compound such as Polyethylene glycol, preferably PEG 200 is preferred over aqueous solutions for storing the swab sample, particularly when the target analyte is sensitive to degradation such as RNA, for 1 hour or more at room-temperature. If the swab sample will be tested within 1 hour, either an aqueous or non-aqueous solution of the quaternary ammonium compound and a halogenated organic compound such as a fluorinated organic compound can be used.

Alternatively, solid quaternary ammonium compound and a halogenated organic compound such as a fluorinated organic compound can be added as a solid directly to an aqueous Virus Transport Medium to give a final concentration of 1 mM to 8 M, more preferably 10 to 600 mM, more preferably 40 to 500 mM and most preferably 400 mM or less of a quaternary ammonium compound and, 1 mM to 8 M, more preferably 20 mM to 1.2 M, more preferably 80 mM to 1 M and most preferably 850 mM or less of a halogenated organic compound such as a fluorinated organic compound preferably Trifluoroacetamide.

### Example 14. Inactivating Virus in a Blood, Plasma, Serum or BAL Sample

To 3 ml of either an aqueous or non-aqueous solution of a quaternary ammonium compound and a halogenated organic compound such as a fluorinated organic compound mixture, preferably both compounds in the range 1 mM to 8 M, e.g. a solution of 10% (vol/vol) Betaine:Trifluoroacetamide (1:2 mol:mol) and 90% (vol/vol) PEG 200, in a 10 ml tube is added 1 ml of blood, serum, plasma or BAL liquid sample from a potentially SARS-CoV-2 infected patient that is required to be tested. The solution is gently shaken and then incubated for 60 minutes at room temperature to allow virus inactivation to occur prior to carrying out an analytical test for the SARS-CoV-2 or other virus, with or without RNA purification, such as LAMP, RT-LAMP, LamPORE test, DNAnudge test, Optigene RT-LAMP, PCR, RT-PCR, geneXpert RT-PCR or BioFire FilmArray RT-PCR or antigen testing such lateral flow.

When the inactivated sample is added to the assay without RNA purification, it is preferable to use a final concentration that does not surpass 10%, more preferably 5%, more preferably 2% and most preferably 1% volume of the final assay volume.

Alternatively, the blood, serum, plasma or BAL liquid sample may be transported and stored in a solution of the quaternary ammonium compound and a halogenated organic compound such as a fluorinated organic compound until needed for an analytical test. A non-aqueous solution of the quaternary ammonium compound and halogenated organic compound, diluted with Polyethylene glycol, preferably PEG 200 is preferred over aqueous solutions for storing the swab sample, particularly when the target analyte is sensitive to degradation such as RNA, for 1 hour or more at room-temperature. If the swab sample will be tested within 1 hour, either an aqueous or non-aqueous solution of the quaternary ammonium compound and a halogenated organic compound such as a fluorinated organic compound can be used.

Alternatively, solid quaternary ammonium compound and a halogenated organic compound such as a fluorinated organic compound can be added as a solid directly to an aqueous Virus Transport Medium to give a final concentration of 1 mM to 8 M, more preferably 10 to 600 mM, more preferably 40 to 500 mM and most preferably 400 mM of a quaternary ammonium compound, preferably Betaine and, 1 mM to 8 M, more preferably 20 mM to 1.2 M, more preferably 80 mM to 1 M and most preferably 850 mM of a halogenated organic compound, preferably Trifluoroacetamide.

### Example 15. Inactivating Samples for Use as a Vaccine

To 150 ml of an aqueous or non-aqueous solution of a quaternary ammonium compound and a mixture of a halogenated organic compound such as a fluorinated organic compound, in a 250 ml flask is added 50 ml of a 10 log 8/ml SARS-CoV-2 sample derived from tissue culture cells such as Vero cells, and then mixed thoroughly with gentle pipetting. The solution is gently shaken and then incubated in the solution of quaternary ammonium compound and halogenated organic compound such as a fluorinated organic compound for 60 minutes at room temperature to allow permanent virus inactivation to occur.

The inactivated virus is then separated from the quaternary ammonium compound and halogenated organic compound such as a fluorinated organic compound solution by size-exclusion chromatography (Spitteler, M A et al. (2011) Vaccine, 29(41), 7182-7187) or other method (Krammer, F. Nature 586, 516-527 (2020). https://doi.org/10.1038/s41586-020-2798-3; Wang et al. Cell. 2020; 182(3):713-721.e9. doi:10.1016/j.cell.2020.06.008), sterile filtered through a 0.22 μm filter, diluted to 10 log 4 inactivated viral particles per ml with sterile saline and alum adjuvant, and 1 ml injected intramuscularly with a 22-25 needle gauge. The injection is repeated at 1 month. (Stern P. L. Annals of Allergy, Asthma & Immunology. 2020 doi: 10.1016/j.anai.2020.01.025).

### Example 16. Disinfecting Blood Products

To 1 Litre of a solution comprising 1 mM to 8 M of a quaternary ammonium compound and 1 mM to 8 M of a halogenated organic compound, preferablyTrifluoroacetamide in an aqueous or non-aqueous solution is added 30 to 300 ml blood or blood products such as serum or an antibody and then mixed thoroughly by gentle pipetting. The solution is very gently mixed and then incubated for 60 minutes at room temperature to allow permanent virus inactivation to occur. The disinfected blood product is then separated from the solution of the quaternary ammonium compound and halogenated organic compound such as a fluorinated organic compound by size-exclusion chromatography as described in Example 15 or molecular weight cut-off (MWCO) centrifugation devices such as 50 kDa MWCO.

### Example 17. Disinfecting and Preserving Samples for Food and Beverage Safety Testing

To 2 ml in a 10 ml tube of a solution comprising 1 mM to 8 M of a quaternary ammonium compound such as Betaine, and 1 mM to 8 M a halogenated organic compound such as Trifluoroacetamide is added to a swab taken from a potentially pathogen contaminated food or beverage sample, food or beverage preparation area, food or beverage preparation equipment, circulating air including air-conditioning unit monitoring filters, or filters for monitoring microbial contamination in liquids and that are used in a food or beverage preparation plant, and batch testing processed food and beverage products. The swab is gently shaken and then incubated in the solution of quaternary ammonium compound and halogenated organic compound such as a fluorinated organic compound for 60 minutes at room temperature to allow microbial inactivation to occur prior to carrying out an analytical test for the presence of the microbial contaminant, with or without analyte purification, such as 3M™ Molecular Detection System, LAMP, RT-LAMP, LamPORE test, PCR, RT-PCR, geneXpert RT-PCR or BioFire FilmArray RT-PCR or antigen testing such lateral flow.

Alternatively, the swab sample may be transported and stored in the solution of quaternary ammonium compound and halogenated organic compound until needed for the analytical test.

A non-aqueous solution of the quaternary ammonium compound and halogenated organic compound, dissolved in for example Polyethylene glycol, preferably PEG 200, is preferred to aqueous solutions for storing the swab sample, particularly when the target analyte is sensitive to degradation such as RNA, for 1 hour or more at room-temperature. If the swab sample will be tested within 1 hour, either an aqueous or non-aqueous solution of the quaternary ammonium compound and halogenated organic compound such as a fluorinated organic compound can be used.

Alternatively, solid quaternary ammonium compounds and halogenated organic compounds can be added as solids directly to an aqueous Virus Transport Medium such as Life Technologies Viral Transport Media (ThermoFisher Scientific) to give a final concentration of 1 mM to 8M, more preferably 10 to 600 mM, more preferably 40 to 500 mM and most preferably 400 mM of a quaternary ammonium compound, preferably Betaine, and 1 mM to 8 M, more preferably 20 mM to 1.2 M, more preferably 80 mM to 1 M and most preferably 850 mM of a halogenated organic compound such as a fluorinated organic compound, preferably Trifluoroacetamide.

### Example 18. Detection of RNA Sequences in a Non-Purified Clinical Sample

In a standard polypropylene PCR capped tube was added 0.5, 1, 2, 5 or 10 ul sputum or swab sample to be tested to a sterile Rnase-free microcentrifuge tube containing 10 μM of oligo (dT), a SARS-CoV-2 target-specific reverse transcriptase primer (e.g. ATATTGCAGCAGTACGCACACA (SEQ ID NO: 1) (Corman, et al. (2020) Detection of 2019 novel coronavirus (2019 nCoV) by real-time RT-PCR. Euro Surveill 2020; 25) or random hexamers and water then added to a final volume of 68 μl. The tube was heated to 70° C. for 1-5 minutes to lyse the sample and denature proteins and template RNA, then 32 ul of reverse transcription reaction was added containing; 20 μl 5× reaction buffer (final concentration 50-400 mM Tris-HCl (pH 7.0-8.8 at 25° C. preferably pH 8.3), 100-500 mM KCl, 50-500 mM DTT), 4 ul MgCl₂ or MgSO₄ to give a final concentration of 0.5-8 mM, preferably 3 mM, 4 μl dNTP (10 mM dATP, dTTP, dCTP, dGTP), 4 μl recombinant Rnasin (Promega Cat. No. N2511) and 4 μl (50-100 units M-MLV RT (H-) Point Mutant™ (Promega Cat. No. M3681), M-MLV RT (Promega Cat. No. M1701), AMV RT (Promega Cat. No. M5101), GoScript™ Reverse Transcriptase (Promega Cat. No. A501D) or ImProm-II™ Reverse Transcriptase (Promega Cat. No. M314B). The reaction was then incubated for 15 minutes each at 37° C., 42° C., 50° C. and 55° C., then placed on ice or frozen until needed for analysis, for example by PCR as described below.

PCR Amplification; The PCR was carried out using 0.5-5 μl of cDNA reaction added to a final volume of 100 μl with final concentration of 15 mM Tris-HCl pH 8.8, 60 mM KCl, 2.5 mM MgCl₂ or MgSO₄, 400 μM each dNTP, 10 μM of each SARS-CoV-2 target-specific primer Forward ACAGGTACGTTAATAGTTAATAGCGT (SEQ ID NO: 2) and Reverse ATATTGCAGCAGTACGCACACA (SEQ ID NO: 1) and 0.4 unit Platinum II Taq™ (ThermoFisher Cat. No. 14966001) or Taq DNA polymerase, for example Taq enzyme (Sigma-Aldrich Cat. No. D1806). Cycle parameters were 94° C.×20 sec, 55° C.×20 sec and 72° C.×30 sec for 30 cycles but were adjusted according to primer sequence and melting temperature. PCR products were visualised following gel electrophoresis and staining with a nucleic acid stain. It will be apparent to one skilled in the field that other types of samples than sputum can be used for example material from clinical swabs, serum, plasma, blood as described elsewhere in the description.

It will also be apparent that there are a great number of reverse transcriptases and DNA polymerases that are commercially available. The most appropriate enzyme can be found empirically by quantifying the amount of either cDNA after the reverse transcriptase reaction or PCR product after PCR amplification. Reverse transcriptases are preferred that have substantial activity in the presence of inhibitors such as heme, proteins, salts, and other well known inhibitors.

### Example 19: Detection of RNA Sequences in a Non-Purified Clinical Sample Using Alternative Polymerases

In a standard polypropylene PCR capped tube was added 0.5, 1, 2, 5 or 10 μl sputum or swab sample to be tested to a sterile Rnase-free microcentrifuge tube containing 200 ng of oligo (dT), a target-specific reverse transcriptase primer (a suitable SARS-CoV-2 reverse transcriptase primer is 10 μM of Reverse ATATTGCAGCAGTACGCACACA (SEQ ID NO: 1)) or random hexamers and water then added to a final volume of 68 μl. The tube was heated to 65° C. for 1 minutes to lyse the sample and denature proteins and template RNA, then 90 μl of reverse transcription reaction was added containing; 20 μl 5×SSIV reaction buffer (ThermoFisher), 5 ul 50-500 mM DTT, optionally 5 μl additional MgCl₂ or MgSO₄ to provide an additional 0.05-4 mM MgCl₂ or MgSO₄ final concentration, 5 μl dNTP (10 mM dATP, dTTP, dCTP, dGTP), 5 μl recombinant Rnasin (ThermoFisher) and 5 μl of SuperScript IV™ 200 u/ul (ThermoFisher Cat. no. 18091050) and water where needed to final volume after 100 μl. Alternatively, SuperScript II™ 200 u/ul (ThermoFisher Cat. no. 18064014) or SuperScript III™ 200 u/ul (ThermoFisher Cat. no. 18080093) can substitute for SuperScript IV™. For SuperScript IV reactions the tubes were incubated for 10 minutes each at 50° C. and 55° C., then placed on ice or frozen until needed, for SuperScript II™ and III reactions the tubes were incubated for 15 minutes each at 37° C., 42° C., 50° C. and 55° C., then placed on ice or frozen until needed. It will be apparent to one skilled in the art that many different types of reverse transcriptases, reaction buffers and reaction temperatures can be used. Reverse transcriptase enzymes are preferred that support higher contaminant concentrations of for example heme, mucin and tannins.

PCR Amplification The PCR was carried out using 1-2 ul of cDNA reaction added to a final volume of 50 μl with final concentration of 15 mM Tris-HCl pH 8.8, 60 mM KCl, 2.5 mM MgCl₂ or MgSO₄, 400 μM each dNTP, 10 μM of each SARS-CoV-2 target-specific primer Forward ACAGGTACGTTAATAGTTAATAGCGT (SEQ ID NO: 2) and Reverse ATATTGCAGCAGTACGCACACA (SEQ ID NO: 1) and 0.4 unit Platinum II Taq™ (ThermoFisher Cat. No. 14966001) or Taq DNA polymerase, for example Taq enzyme (Sigma-Aldrich Cat. No. D1806). Cycle parameters were 94° C.×20 sec, 55° C.×20 sec and 72° C.×30 sec for 30 cycles but were adjusted according to primer sequence and Tm. PCR products were visualised following gel electrophoresis and staining with a nucleic acid stain.

### Example 20. RT-PCR from a Non-Purified Sample Using Alternative Reaction Buffer Conditions

Reverse Transcription: 0.5, 1, 2, 5, 7.5 or 10 μl sputum or swab sample to be tested was added to a sterile Rnase-free microcentrifuge tube containing the reaction mixture with the following final component concentrations: 50-200 mM Tris-HCl (pH 8.4 at 24° C.), 75 mM KCl, 0.5-5 mM MgCl₂, MgSO₄ or MnCl₂ preferably 1.3 mM, 10 mM DTT, 1 mM dNTP's, 110 ng of oligonucleotide reverse transcriptase primer (a suitable SARS-CoV-2 reverse transcriptase primer is 10 uM of Reverse ATATTGCAGCAGTACGCACACA (SEQ ID NO: 1) and 100 units of SuperScript III™ 200 u/ul (Thermo Cat. no. 18080093) or M-MLV RT (H-) Point Mutant (Promega Cat. No. M3681). Water was used to bring the final volume to 100 μl. The reaction was allowed to proceed for 10 min at 55° C. The cDNA can then be purified by for example, ethanol precipitation, Centricon-50 filtration (according to manufacturer's instructions) or used directly in a qPCR reaction as follows.

PCR Amplification The PCR was carried out using 1-2 μl of cDNA reaction added to a final volume of 100 μl with final concentration of 15 mM Tris-HCl pH 8.8, 60 mM KCl, 2.5 mM MgCl₂ or MgSO4, 400 μM each dNTP, 10 μM of each SARS-CoV-2 target-specific primer Forward ACAGGTACGTTAATAGTTAATAGCGT (SEQ ID NO: 2) and Reverse ATATTGCAGCAGTACGCACACA (SEQ ID NO: 1) and 0.4 unit Platinum II Taq™ (ThermoFisher Cat. No. 14966001) or Taq DNA polymerase, for example Taq enzyme (Sigma-Aldrich Cat. No. D1806) PCR was carried out with an initial 95° C.×3 min and then 45 cycles of 95° C.×15 sec, 58° C.×30 sec prior to analysis by agarose gel electrophoresis. Real-time qPCR can be carried out by using a suitable real-time labelled probe such as ACACTAGCCATCCTTACTGCGCTTCG (SEQ ID NO: 3) [5′] Fam, [3′] BHQ-1, using either Roche Light Cycler 48011 or Applied Biosystems ViiA7 instruments.

### Example 21. Detection of RNA Sequences in a Non-Purified Clinical Sample Treated with a Deep Eutectic Solvent

A sputum sample was prepared by adding 9 volumes of Trimethylglycine:Trifluoroacetamide (1:2 mol:mol) to 1 volume of sputum or swab sample and mixing for 60 minutes to inactivate any viruses present in the sputum sample. Alternatively, either 1, 2, 3, 5 or 9 volumes of Trimethylglycine:Trifluoroacetamide (1:2 mol:mol) are added to 1 volume of sputum or swab sample in a sterile polypropylene tube followed by mixing for 5 to 60 minutes to inactive any viruses present in the sample.

The reverse transcription was carried out using 0.5, 1, 2, 5, 7.5 or 10 μl of the DES:sputum sample added to a final volume of 100 μl reaction containing 200 ng of oligo (dT), a target-specific reverse transcriptase primer (a suitable SARS-CoV-2 reverse transcriptase primer is 10 μM of Reverse ATATTGCAGCAGTACGCACACA (SEQ ID NO: 1)), or random hexamers and water then added to a final volume of 68 ul. The tube was heated to 65-95° C. for 1-10 minutes to lyse the sample and denature proteins and template RNA, then 90 μl of reverse transcription reaction was added containing; 20 μl 5×SSIV reaction buffer, 5 μl 50-500 mM DTT, optionally 5 μl additional MgCl₂ or MgSO₄ to provide an additional 0.05-4 mM MgCl₂ or MgSO₄ final concentration, 5 μl dNTP (10 mM dATP, dTTP, dCTP, dGTP), 5 μl recombinant Rnasin (ThermoFisher) and 5 μl of SuperScript IV™ 200 u/ul (Thermo Cat. no. 18091050) and water where needed to final volume after 100 μl. Alternatively, SuperScript II™ 200 u/ul (Thermo Cat. no. 18064014) or SuperScript III™ 200 u/ul (Thermo Cat. no. 18080093) can substitute for SuperScript IV. For SuperScript IV™ reactions the tubes were incubated for 10 minutes each at 50° C. and 55° C., then placed on ice or frozen until needed, for SuperScript II and III reactions the tubes were incubated for 15 minutes each at 37° C., 42° C., 50° C. and 55° C., then placed on ice or frozen until needed. It will be apparent to one skilled in the art that many different types of reverse transcriptases, buffers and reaction temperatures can be used. Enzymes are preferred that support higher contaminant concentrations and such enzymes can be determined empirically.

PCR Amplification. The PCR was carried out using 1-2 μl of cDNA reaction added to a final volume of 50 μl with final concentration of 15 mM Tris-HCl pH 8.8, 60 mM KCl, 2.5 mM MgCl₂ or MgSO₄, 400 μM each dNTP, 10 μM of each SARS-CoV-2 target-specific primer Forward ACAGGTACGTTAATAGTTAATAGCGT (SEQ ID NO: 2) and Reverse ATATTGCAGCAGTACGCACACA (SEQ ID NO: 1) and 0.4 unit Platinum II Taq™ (ThermoFisher Cat. No. 14966001) or Taq DNA polymerase, for example Taq enzyme (Sigma-Aldrich Cat. No. D1806). Cycle parameters were 94° C.×20 sec, 55° C.×20 sec and 72° C.×30 sec for 30 cycles but were adjusted according to primer sequence and Tm. PCR products were visualised following gel electrophoresis and staining with a nucleic acid stain.

Alternatively, Trimethylglycine:Trifluoroacetamide (1:2 mol:mol) can be replaced with Trimethylglycine:Glycerol (1:2 mol:mol), Choline chloride:urea (1:2 mol:mol), Choline chloride:Glycerol (1:2 mol:mol), Trimethylglycine:Xylitol (1:2 mol:mol), Choline chloride:Xylitol (1:2 mol:mol), Trimethylglycine:Sorbitol (1:2 mol:mol), Choline chloride: Sorbitol (1:2 mol:mol), Trimethylglycine:D+Galactose (1:2 mol:mol), Choline chloride: D+Galactose (1:2 mol:mol), Trimethylglycine:Trifluoroacetamide (1:2 mol:mol) containing 10 mM ZnSO4, Trimethylglycine:Trifluoroacetamide (1:2 mol:mol) containing 20, 50,70, 90, or 95% PEG200, Trimethylglycine:Trifluoroacetamide (1:2 mol:mol) containing 20, 50,70, 90, or 95% Tetraethylene Glycol, Trimethylglycine:Trifluoroacetamide (1:2 mol:mol) containing 20, 50, 70, 90, or 95% Tetraethylene glycol monomethyl ether, Trimethylglycine:Trifluoroacetamide (1:2 mol:mol) containing 20, 50, 70, 90, or 95% Tetraethylene glycol dimethyl ether, Trimethylglycine:Trifluoroacetamide (1:2 mol:mol) containing 20, 50,70, 90, or 95% water, Trimethylglycine:Trifluoroacetamide (1:2 mol:mol) containing 50 mM-3.6 M final concentration Formic acid or 100 mM-13 M Acetic acid.

### Example 22. Direct PCR of a DNA Analyte from a Non-Purified Sample

The PCR was carried out using 0.5, 1, 2, 5, 7.5 or 10 μl sputum or swab sample added to a final volume of 50 ul of PCR reaction with final concentration of 15 mM Tris-HCl pH 8.8, 60 mM KCl, 2.5 mM MgCl₂ or MgSO₄, 400 μM each dNTP, 10 pmol of each primer Forward and Reverse and 0.4 unit Platinum II Taq™ (ThermoFisher Cat. No. 14966001) or Taq DNA polymerase, for example Taq enzyme (Sigma-Aldrich Cat. No. D1806). Cycle parameters were 94° C.×5 min, followed by 30 cycles of 94° C.×20 sec, 55° C.×20 sec and 72° C.×30 sec but temperatures were adjusted according to primer sequence and melting temperature. PCR products were visualised following gel electrophoresis and staining with a nucleic acid stain.

### Example 23. Direct PCR of a DNA Analyte from a Non-Purified Sample in a Deep Eutectic Solvent (DES)

A sputum sample was prepared by adding 9 volumes of Trimethylglycine:Trifluoroacetamide (1:2 mol:mol) to 1 volume of sputum or swab sample and mixing for 60 minutes to inactivate any viruses present in the sputum sample.

Alternatively, a sputum sample is prepared by adding, 2, 3, 5 or 9 volumes of Trimethylglycine:Trifluoroacetamide (1:2 mol:mol) to 1 volume of sputum or swab sample followed by mixing for 5 to 60 minutes in a sterile polypropylene microcentrifuge tube

The PCR was carried out using 0.5, 1, 2, 5, 7.5 or 10 μl of the DES:sputum sample added to a final volume of 50 ul of PCR reaction containing 15 mM Tris-HCl pH 8.8, 60 mM KCl, 2.5 mM MgCl₂ or MgSO₄, 400 μM each dNTP, 10 pmol of each primer Forward and Reverse and 0.4 unit Platinum II Taq™ (ThermoFisher Cat. No. 14966001) or Taq DNA polymerase, for example Taq enzyme (Sigma-Aldrich Cat. No. D1806). Cycle parameters were 94° C.×5 min, followed by cycles of 94° C.×20 sec, 55° C.×20 sec and 72° C.×30 sec but temperatures were adjusted according to primer sequence and Tm. PCR products were visualised following gel electrophoresis and staining with a nucleic acid stain.

There are a wide range of different lysis enhancers such as reductants including DTT, TCEP and beta-mercpatoethanol that can be added to a final concentration of 5-100 mM, or solvents such as DMSO that can be added up to 15% final volume. Reverse transcriptase inhibitors that can be include BSA, spermine, PVP, and heparinise.

Alternatively, Trimethylglycine:Trifluoroacetamide (1:2 mol:mol) can be replaced with Trimethylglycine:Glycerol (1:2 mol:mol), Choline chloride:urea (1:2 mol:mol), Choline chloride:Glycerol (1:2 mol:mol), Trimethylglycine:Xylitol (1:2 mol:mol), Choline chloride:Xylitol (1:2 mol:mol), Trimethylglycine:Sorbitol (1:2 mol:mol), Choline chloride: Sorbitol (1:2 mol:mol), Trimethylglycine:D+Galactose (1:2 mol:mol), Choline chloride: D+Galactose (1:2 mol:mol), Trimethylglycine:Trifluoroacetamide (1:2 mol:mol) containing 10 mM ZnSO₄, Trimethylglycine:Trifluoroacetamide (1:2 mol:mol) containing 20, 50,70, 90, or 95% PEG200, Trimethylglycine:Trifluoroacetamide (1:2 mol:mol) containing 20, 50,70, 90, or 95% Tetraethylene Glycol, Trimethylglycine:Trifluoroacetamide (1:2 mol:mol) containing 20, 50,70, 90, or 95% Tetraethylene glycol monomethyl ether, Trimethylglycine:Trifluoroacetamide (1:2 mol:mol) containing 20, 50,70, 90, or 95% Tetraethylene glycol dimethyl ether, Trimethylglycine:Trifluoroacetamide (1:2 mol:mol) containing 20, 50,70, 90, or 95% water, Trimethylglycine:Trifluoroacetamide Trimethylglycine:Trifluoroacetamide (1:2 mol:mol) containing 50 mM-3.6M final concentration Formic acid or 100 mM to 13 M Acetic acid.

### Example 24. Direct PCR of a DNA Analyte from a Non-Purified Sample Using Lysis Enhancers

The PCR was carried out using 0.5, 1, 2, 5, 7.5 or 10 μl sputum or swab sample added to a final volume of 50 ul with final concentration of 0.1-100 mM of a non-ionic detergent such as Tween-20, Tween-40, Tween-60, Tween-80, Digitonin, NP-40, Nonidet P-40, Triton X-100, Brij-35, Igepal-CA630, Saponin and/or Tergitol, 15 mM Tris-HCl pH 8.8, 60 mM KCl, 2.5 mM MgCl₂ or MgSO₄, 400 μM each dNTP, 10 pmol of each primer Forward and Reverse and 0.4 unit Platinum II Taq™ (ThermoFisher Cat. No. 14966001) or Taq DNA polymerase, for example Taq enzyme (Sigma-Aldrich Cat. No. D1806). Cycle parameters were 94° C.×5 min, followed by cycles of 94° C.×20 sec, 55° C.×20 sec and 72° C.×30 sec but temperatures were adjusted according to primer sequence and Tm. PCR products were visualised following gel electrophoresis and staining with a nucleic acid stain.

There are a wide range of different lysis enhancers such as non-ionic detergents, reductants including DTT that can be added to a final concentration of 1-100 mM, or solvents such as DMSO that can be added up to 10% final volume.

### Example 25. Real-Time RT-PCR Assays for the Detection of SARS-CoV-2 Protocol

Using the protocol of Corman, et al. (2020) Detection of 2019 novel coronavirus (2019 nCoV) by real-time RT-PCR. Euro Surveill 2020; 25. Instead of 5 μl purified RNA added to the RT-qPCR reaction, the RNA was substituted with 1-5 μl sputum or swab sample and made up, if necessary, to a final volume with 5 μl with water. A suitable SARS-CoV-2 reverse transcriptase primer is 10 μM of Reverse ATATTGCAGCAGTACGCACACA (SEQ ID NO: 1).

Alternatively, the sputum or swab sample can be replaced with 1-5 μl of swab material prepared by adding 10-20 μl of water to the swab (following sample collection) directly using a pipette to the swab brushes, pipetting up and down 5 times and then pipetting off the liquid containing the sample and transferring 5 μl to the reverse reaction as described by the authors. Reverse transcription and qPCR were carried out according to Corman et al. Suitable PCR primers are 10 μM of each SARS-CoV-2 target-specific primer Forward ACAGGTACGTTAATAGTTAATAGCGT (SEQ ID NO: 2) and Reverse ATATTGCAGCAGTACGCACACA (SEQ ID NO: 1).

### Example 26. Detection of Viral Sequences in a Non-Purified but Anion Exchange Bead Concentrated Sample

A 200 μl sputum or swab sample from a COVID-19 patient was mixed with 5-50 mg of a weak anion exchange resin Amberlite™ IRA743 (Sigma-Aldrich 216445) and incubated for 30 minutes at room-temperature with slow shaking to allow negatively charged virus capture, followed by 15 minutes without mixing to allow the resin to separate under unit gravity, the supernatant was then removed with a pipette and the resin bound to the virus was treated with 0.1 M NaCl to remove the virus from the resin. 0.5, 1, 2, 5 or 10 μl of the virus in 0.1 M NaCl sample to a 10, 20, 25, 50 or 100 μl reverse transcription as set out in Examples 18 to 20. Alternatively, 1-50 mg weak anion exchange resin can be added directly to the reverse transcription reaction as a source of virus.

### Example 27. Detection of Viral Sequences in a Non-Purified but Protein-A Bead Concentrated Sample

A 200 μl sputum or swab sample from a COVID-19 patient was mixed with 0.05 g of IgG binding Protein A resin beads (Sigma-Aldrich Cat. No. LSKMAGA02) pre-loaded with anti-SARS-CoV-2 antibodies, preferably directed to the spike protein (e.g. Human Anti-SARS-CoV-2 S1 Monoclonal antibody, clone BIB112, Creative Diagnostics Cat. No. CABT-CS031) and incubated in PBS or TBST for 30 minutes at 4° C. or room-temperature with slow shaking, followed by 15 minutes without mixing to allow the resin to separate under unit gravity, the supernatant was then removed with a pipette and the resin bound to the virus was added directly to a 10, 20, 25, 50 or 100 μl reverse transcription as set out for example in Example 18 to 20.

### Example 28. Detection of Viral Sequences in an Aqueous Depleted Sample

A 200 μl sputum or swab sample from a COVID-19 patient was mixed with 10-250 mg of a drying solid phase such as Molecular sieves 4 A, silica gel, dry polyacrylamide, dry polyacrylic acid, super adsorbent polymers and incubated for 30 minutes at room-temperature to allow bead swelling and aqueous volume reduction, 0.5, 1, 2, 5 or 10 ul of the supernatant bearing the concentrated free virus was added to a 10, 20, 25, 50 or 100 ul reverse transcription reaction. It is desirable that at least 80-95% of the aqueous volume is adsorbed by the solid phase and that the remaining liquid, bearing the concentrated virus can be removed simply by pipetting on top of, or between the solid phase material.

### Example 29. Detection of Viral Sequences in a Non-Purified Agglutinated Sample

A 200 μl sputum or swab sample from a COVID-19 patient was mixed with 25 ul of 0.1M NaOAc pH 6.0 and incubated for 15 minutes at room-temperature to allow virus agglutination, followed by centrifugation at 15,000×g for 15 minutes at 4° C. The pellet was taken up in 100 μl of water and 0.5, 1, 2, 5 or 10 μl of the water bearing the virus was added to a 10, 20, 25, 50 or 100 μl reverse transcription reaction.

### Example 30. Using the 10× Genomics Single Cell 3′ Library Kit

As there is insufficient RNA or DNA in a single virus to reproducibly detect it using current scRNA-seq, 1-500 viable tissue culture cells expressing large amounts of the SARS-CoV-2 binding receptor ACE2 (Mossel et al., (2005) J Virol. 79(6): 3846-3850) were mixed gently with 10-1000 ul of sputum or swab sample suspected of containing the SARS-CoV-2 virus, for 15 minutes at 4° C. or room-temperature in PBS or TBST containing 10% FCS to allow virus binding to the ACE2 receptor on the cell surface, and therefore become immobilised and associated with a specific cell. The cells were then briefly washed in PBS containing 10% FCS to remove non-bound virus, optionally a virus-cell cross-linking step is carried out to immobilise the 2 together, followed by a wash step, and the process repeated with 1-100,000 other samples e.g. 4, 9, 99, 999 or 9,999 other samples, each time the ACE2 expressing cells were pooled with the other pools so that 5000 cells representing 100 samples were obtained and then processed according to manufacturers (10× Genomics) instructions.

Single cell suspensions were loaded onto the 10× Chromium device using the 10× Genomics Single Cell 3′ Library Kit v2 (10× Genomics; PN-120237, PN-120236, PN-120262) to generate cell and gel bead emulsions, followed by reverse transcription, cDNA amplification, and sequencing library preparation following the manufacturers' instructions. The resulting libraries were sequenced with one sample per lane using the NextSeq500 (Illumina; high-output mode, paired-end 26×49 bp) or with two samples per lane using the HiSeq4000 (Illumina; paired-end 26×74 bp). Barcodes specific to each of the patient samples were retrieved by bioinformatics and the presence of SARS-CoV-2 sequences associated with each barcoded sequence provided the infection status of each patient.

### Example 31. Using the Takara Reverse Transcriptase Kit SMARTer™

In a standard 25 μl polypropylene PCR capped tube was added 0.5, 1, 2, 5 or 10 μl sputum or swab sample to be tested for SARS-CoV-2 to a 10, 20, 25, 50 or 100 ul reverse transcription reaction according to SMARTer® PCR cDNA Synthesis Kit according to manufacturer's instructions (Takara Cat. No. 634926) including 3′ SMART CDS Primer IIA (12 μM).

Alternatively, the sputum or swab sample is first treated with a 1:1, 2:1, 3:1, 5:1 or 9:1 vol:vol of Trimethylglycine:Trifluoroacetamide (1:2 mol:mol), incubated at room-temperature for 1 hour before 0.5, 1, 2, 5 or 10 μl added to a SMARTer® PCR cDNA Synthesis Kit. Alternatively, Trimethylglycine:Glycerol (1:2 mol:mol), Choline chloride:urea (1:2 mol:mol), Choline chloride:Glycerol (1:2 mol:mol), Trimethylglycine:Xylitol (1:2 mol:mol), Choline chloride:Xylitol (1:2 mol:mol), Trimethylglycine:Sorbitol (1:2 mol:mol), Choline chloride: Sorbitol (1:2 mol:mol), Trimethylglycine:D+Galactose (1:2 mol:mol), Choline chloride: D+Galactose (1:2 mol:mol), Trimethylglycine:Trifluoroacetamide (1:2 mol:mol) containing 10 mM ZnSO₄, Trimethylglycine:Trifluoroacetamide (1:2 mol:mol) containing 20, 50, 70, 90 or 95% PEG200, Trimethylglycine:Trifluoroacetamide (1:2 mol:mol) containing 20, 50, 70, 90 or 95% Tetraethylene Glycol, Trimethylglycine:Trifluoroacetamide (1:2 mol:mol) containing 20, 50, 70, 90 or 95% Tetraethylene glycol monomethyl ether, Trimethylglycine:Trifluoroacetamide (1:2 mol:mol) containing 20, 50, 70, 90 or 95% Tetraethylene glycol dimethyl ether, Trimethylglycine:Trifluoroacetamide (1:2 mol:mol) containing 20, 50, 70, 90 or 95% water, Trimethylglycine:Trifluoroacetamide (1:2 mol:mol) containing 50 mM-3.6 M final concentration Formic acid or 100 mM-13 M Acetic acid.

### Example 32. Using PrimeDirect™ Probe RT-qPCR Mix

In a standard 25 μl polypropylene PCR capped tube was added 0.5, 1, 2, 5 or 10 ul sputum or swab sample to be tested for SARS-CoV-2 to a 10, 20, 25, 50 or 100 μl PrimeDirect™ Probe RT-qPCR Mix reverse transcription reaction according to manufacturer's instructions (Takara Cat. No. RR650A). Alternatively, the sputum is first treated with a 1:1, 2:1, 3:1, 5:1 or 9:1 vol:vol ofTrimethylglycine:Trifluoroacetamide (1:2 mol:mol), incubated at room-temperature for 1 hour before 0.5, 1, 2, 5 or 10 ul added to a 100 ul PrimeDirect™ Probe RT-qPCR Mix reverse transcription reaction. Alternatively, Trimethylglycine:Glycerol (1:2 mol:mol), Choline chloride:urea (1:2 mol:mol), Choline chloride:Glycerol (1:2 mol:mol), Trimethylglycine:Xylitol (1:2 mol:mol), Choline chloride:Xylitol (1:2 mol:mol), Trimethylglycine:Sorbitol (1:2 mol:mol), Choline chloride: Sorbitol (1:2 mol:mol), Trimethylglycine:D+Galactose (1:2 mol:mol), Choline chloride: D+Galactose (1:2 mol:mol), Trimethylglycine:Trifluoroacetamide (1:2 mol:mol) containing 10 mM ZnSO4, Trimethylglycine:Trifluoroacetamide (1:2 mol:mol) containing 20, 50,70, 90, or 95% PEG200, Trimethylglycine:Trifluoroacetamide (1:2 mol:mol) containing 20, 50,70, 90, or 95% Tetraethylene Glycol, Trimethylglycine:Trifluoroacetamide (1:2 mol:mol) containing 20, 50,70, 90, or 95% Tetraethylene glycol monomethyl ether, Trimethylglycine:Trifluoroacetamide (1:2 mol:mol) containing 20, 50, 70, 90, or 95% Tetraethylene glycol dimethyl ether, Trimethylglycine:Trifluoroacetamide (1:2 mol:mol) containing 20, 50, 70, 90, or 95% water, Trimethylglycine:Trifluoroacetamide (1:2 mol:mol) containing 50 mM-3.6 M final concentration Formic acid or 100 mM-13 M Acetic acid.

### Example 33. Using the Thermo Cells-to-cDNA Reverse Transcriptase Kit

In a standard 25 μl polypropylene PCR capped tube was added 0.5, 1, 2, 5 or 10 μl sputum or swab sample to be tested for SARS-CoV-2 to a 10, 20, 25, 50 or 100 ul Cells-to-cDNA II™ RT Mix reverse transcription reaction according to manufacturer's instructions (ThermoFisher Cat. No. AM1722). Alternatively, the sputum is first treated with a 1:1, 2:1, 3:1, 5:1 or 9:1 vol:vol ofTrimethylglycine:Trifluoroacetamide (1:2 mol:mol), incubated at room-temperature for 1 hour before 0.5, 1, 2, 5 or 10 ul added to a 100 ul Cells-to-cDNA II™ RT Mix Probe RT-qPCR Mix reverse transcription reaction.

Alternatively, Trimethylglycine:Glycerol (1:2 mol:mol), Choline chloride:urea (1:2 mol:mol), Choline chloride:Glycerol (1:2 mol:mol), Trimethylglycine:Xylitol (1:2 mol:mol), Choline chloride:Xylitol (1:2 mol:mol), Trimethylglycine:Sorbitol (1:2 mol:mol), Choline chloride: Sorbitol (1:2 mol:mol), Trimethylglycine:D+Galactose (1:2 mol:mol), Choline chloride: D+Galactose (1:2 mol:mol), Trimethylglycine:Trifluoroacetamide (1:2 mol:mol) containing 10 mM ZnSO₄, Trimethylglycine:Trifluoroacetamide (1:2 mol:mol) containing 20, 50,70, 90, or 95% PEG200, Trimethylglycine:Trifluoroacetamide (1:2 mol:mol) containing 20, 50,70, 90, or 95% Tetraethylene Glycol, Trimethylglycine:Trifluoroacetamide (1:2 mol:mol) containing 20, 50, 70, 90, or 95% Tetraethylene glycol monomethyl ether, Trimethylglycine:Trifluoroacetamide (1:2 mol:mol) containing 20, 50, 70, 90, or 95% Tetraethylene glycol dimethyl ether, Trimethylglycine:Trifluoroacetamide (1:2 mol:mol) containing 20, 50, 70, 90, or 95% water, Trimethylglycine:Trifluoroacetamide (1:2 mol:mol) containing 50 mM-3.6 M final concentration Formic acid or 100 mM-13 M Acetic acid.

### Example 34. Inactivating M13 Bacteriophage with Dilutions of Betaine:Trifluoroacetamide

To 10 μl of concentrated M13 bacteriophage stock (10 log 9/ml) was added 90 μl of either:
(i) Betaine:Trifluoroacetamide 1:2 mol:mol,
(ii) 10% Betaine:Trifluoroacetamide 1:2 mol:mol/90% water,
(iii) 5% Betaine:Trifluoroacetamide 1:2 mol:mol/95% water,
(iv) 10% Betaine:Trifluoroacetamide 1:2 mol:mol/90% PEG 200,
(v) 10% Betaine:Trifluoroacetamide 1:2 mol:mol/90% Tetraethylene glycol or
(vi) 10% Betaine:Trifluoroacetamide 1:2 mol:mol/45% Tetraethylene glycol/45% Tetraethylene glycol monomethyl ether.

The mixtures were mixed well by vortexing and then incubated in total for 10 minutes at 20° C. before the addition and mixing of 990 μl of growth media LB. 10 μl of this diluted phage mixture was removed and added and mixed well with 90 μl of LB. Finally, 10 μl of this dilution was taken and mixed with 100 ul of freshly grown E. coli XL-2 bacteria.

The phage/bacteria was immediately added to 5 ml of Top Agar containing 4 μl IPTG and 40 μl X-gal to allow plaques to be visualised by their blue colour. The entire tube contents were immediately poured onto a 20 ml pre-warmed agar plate and incubated for 5 hours or more at 37° C. before the number of plaques per plate were counted using an A1 Python program.

The percentage M13 bacteriophage knockdown were as follows:

| Treatment | Inactivation |
|---|---|
| Betaine:Trifluoroacetamide 1:2 mol:mol | 98.2% |
| 10% Betaine:Trifluoroacetamide 1:2 mol:mol/90% water | 41% |
| 5% Betaine:Trifluoroacetamide 1:2 mol:mol/95% water | 34% |
| 10% Betaine:Trifluoroacetamide 1:2 mol:mol/90% PEG 200 | 87.2% |
| 10% Betaine:Trifluoroacetamide 1:2 mol:mol/90% Tetraethylene glycol | 92% |
| 10% Betaine:Trifluoroacetamide 1:2 mol:mol/45% Tetraethylene glycol/45% | 100% |
| Tetraethylene glycol monomethyl ether | |
| 10% Betaine:Trifluoroacetamide 1:2 mol:mol/20% Tetraethylene glycol/80% | 100% |
| Tetraethylene glycol monomethyl ether | |
| 10% Betaine:Trifluoroacetamide 1:2 mol:mol/40% Tetraethylene glycol/60% | 100% |
| Tetraethylene glycol monomethyl ether | |
| 10% Betaine:Trifluoroacetamide 1:2 mol:mol/60% Tetraethylene glycol/40% | 100% |
| Tetraethylene glycol monomethyl ether | |
| 10% Betaine:Trifluoroacetamide 1:2 mol:mol/80% Tetraethylene glycol/20% | 100% |
| Tetraethylene glycol monomethyl ether | |

The LD50, i.e. the amount which inactivated 50% of the M13 phage, for each reagent was:
30% aqueous Betaine:Trifluoroacetamide 1:2 mol:mol
35% aqueous 10% Betaine:Trifluoroacetamide 1:2 mol:mol/90% PEG 200.

The results illustrate that mixtures of quaternary ammonium compounds and halogenated organic compounds are effective for inactivating viruses, and that inactivation efficacy is maintained when the mixtures are diluted, in particular with a polyethylene glycol or an alkyl ether of a polyethylene glycol.

### Example 35. Killing E. coli with Dilutions of Betaine:Trifluoroacetamide

To 1 μl of E. coli JM109 (Amp+) (10 log 9/ml) was added 20 μl of either:
(i) Betaine:Trifluoroacetamide 1:2 mol:mol,
(ii) 10% Betaine:Trifluoroacetamide 1:2 mol:mol/90% water,
(iii) 5% Betaine:Trifluoroacetamide 1:2 mol:mol/95% water, or
(iv) 10% Betaine:Trifluoroacetamide 1:2 mol:mol/90% PEG 200.

The resulting mixtures were mixed well by vortexing and then incubated in total for 30 minutes at 20° C. before the addition and mixing of 1.5 ml of growth media LB. 20 μl of this diluted bacteria mixture was removed and added to a 20 ml pre-warmed agar plate containing 100 μg/ml ampicillin and incubated overnight at 37° C. before the number of colonies per plate were counted using an A1 Python program.

The bacterial killing results were:

| Treatment | Killing |
|---|---|
| Betaine:Trifluoroacetamide 1:2 mol:mol | 100% |
| 10% Betaine:Trifluoroacetamide 1:2 mol:mol/90% water | 100% |
| 5% Betaine:Trifluoroacetamide 1:2 mol:mol/95% water | 69% |
| 10% Betaine:Trifluoroacetamide 1:2 mol:mol/90% PEG 200 | 100% |

The LD 50 of each reagent, i.e. the amount which killed 50% of the bacteria was:
3.2% aqueous Betaine:Trifluoroacetamide 1:2 mol:mol
3.7% aqueous 10% Betaine:Trifluoroacetamide 1:2 mol:mol/90% PEG 200.

The results illustrate that mixtures of quaternary ammonium compounds and halogenated organic compounds are effective for killing bacteria, and that inactivation efficacy is maintained when the mixtures are diluted, in particular with a polyethylene glycol.

### Example 36. RNA Stabilisation in Animal Tissue Samples Using PEG 200 Dilutions of Betaine:Trifluoroacetamide

To 400 μl of:
(i) Betaine:Trifluoroacetamide (1:2 mol:mol) or
(ii) 10% Betaine:Trifluoroacetamide (1:2 mol:mol)/90% PEG 200 (TCI, Belgium) in a standard 1.5 ml polypropylene microcentrifuge tube was added 5 mg rat liver sample and pre-incubated for 20 minutes at room temperature to allow stabilisation and/or fixation. The sample can then be incubated at 20° C. for 5 days prior to recovery of the tissue sample with forceps, followed by RNA purification.

To perform the RNA purification, the sample was added to a fresh tube containing 350 μl of Lysis buffer RLT, and the tissue homogenised according to manufacturer's instructions (Rneasy Mini Kit, Cat. No. 74106, Qiagen, Germany). 300 μl portions of the lysate were then purified immediately according to manufacturer's instructions and eluted into 50 μl of water. 300 ng of the RNA was electrophoresed in a 1% agarose 0.5×TAE gel and imaged using ethidium bromide and a UV lamp. It was found that the RNA quality of the PEG 200 diluted sample was slightly less than the sample stored in 100% Betaine:Trifluoroacetamide but sufficient for molecular tests such as RT-PCR. Results are set out in FIG. 2.

These results show that RNA may be satisfactorily stabilised even by dilutions of a mixture of a quaternary ammonium compound and a halogenated organic compound. Accordingly, detection of viruses in a sample will be possible even when those viruses have been inactivated using the diluted mixture.

### Example 37. RNA Stabilisation in Tissue Culture Cell Samples Using PEG 200 Dilutions of Betaine:Trifluoroacetamide at Extreme Temperatures

To 400 μl of each of:
i) Betaine:Trifluoroacetamide (1:2 mol:mol);
ii) 50% Betaine:Trifluoroacetamide (1:2 mol:mol)/50% PEG 200;
iii) 30% Betaine:Trifluoroacetamide (1:2 mol:mol)/70% PEG 200;
iv) 10% Betaine:Trifluoroacetamide (1:2 mol:mol)/90% PEG 200
   in respective 1.5 ml polypropylene tubes was added 5 mg MKN tissue culture cell pellet and incubated at 60° C. for 20 hours followed by RNA purification as set out in Example 36.

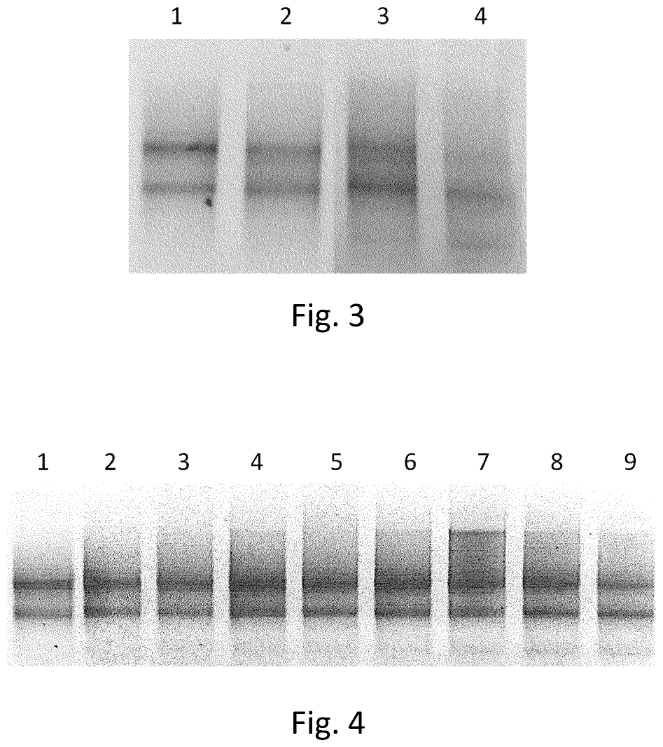

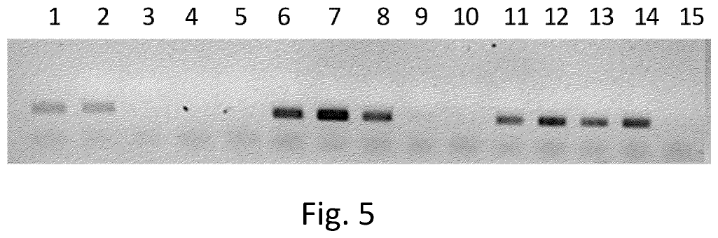

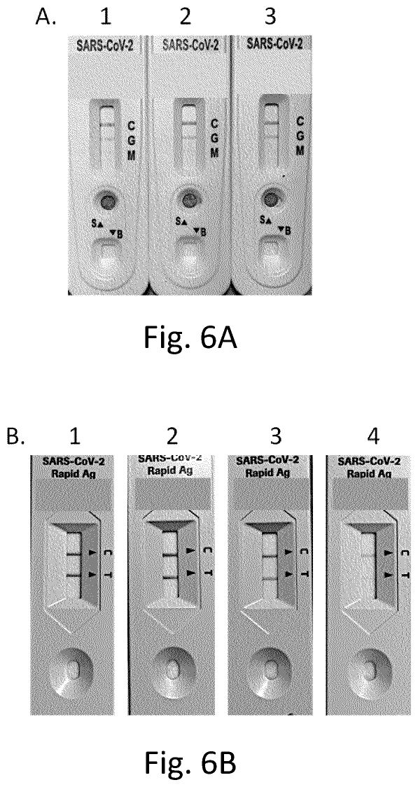

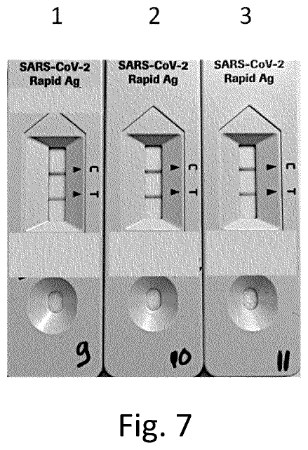

It was found that the RNA quality was best in pure 100% Betaine:Trifluoroacetamide, slightly less good in 50%, less good in 30% and least good in 10% Betaine:Trifluoroacetamide but all RNAs were of sufficient quality for molecular tests such as RT-PCR. Results are set out in FIG. 3.

### Example 38. RNA Stabilisation in Tissue Culture Cell Samples Using PEG 200 Dilutions of Betaine:Trifluoroacetamide at Extreme Temperatures

To 400 μl of:
i) Betaine:Trifluoroacetamide (1:2 mol:mol); or
ii) 88% Betaine:Trifluoroacetamide (1:2 mol:mol)/12% PEG 200; or
iii) 77% Betaine:Trifluoroacetamide (1:2 mol:mol)/23% PEG 200; or
iv) 66% Betaine:Trifluoroacetamide (1:2 mol:mol)/34% PEG 200; or
v) 58% Betaine:Trifluoroacetamide (1:2 mol:mol)/42% PEG 200; or
vi) 46% Betaine:Trifluoroacetamide (1:2 mol:mol)/54% PEG 200, or
vii) 36% Betaine:Trifluoroacetamide (1:2 mol:mol)/64% PEG 200; or
viii) 26% Betaine:Trifluoroacetamide (1:2 mol:mol)/74% PEG 200, or
ix) 16% Betaine:Trifluoroacetamide (1:2 mol:mol)/84% PEG 200
   in a 1.5 ml polypropylene tube was added 5 mg MKN tissue culture cell pellet and incubated at 37° C. for 13 days followed by RNA purification as set out in Example 36.

It was found that the RNA quality was best in pure 100% Betaine:Trifluoroacetamide, and reasonable for all PEG 200 dilutions. Results are set out in FIG. 4. The RNA quality of the samples stored in the PEG 200 dilutions would be adequate for allowing detection of viruses. The duration of the storage and the storage temperature were both longer than what would typically be encountered during the processing of clinical samples e.g. for mass virus testing.

### Example 40. Effect of PEG 200 Diluted Betaine:Trifluoroacetamide on PCR Reaction

The PCR was carried out in a final volume of 25 μl with final concentration of 15 mM Tris-HCl pH 8.8, 60 mM KCl, 2.5 mM MgCl₂, 400 μM each dNTP, 10 pmol of each primer BMV F (CTATCACCAAGATGTCTTCG (SEQ ID NO: 4) and BMV R (GAGCCCCAGCGCACTCGGTC (SEQ ID NO: 5)) and 1 unit Taq DNA polymerase (Sigma-Aldrich, UK). 1 μl of template BMV cDNA (Promega, UK) was added per reaction.

The following compositions were added to the PCR mixture
(i) Betaine:Trifluoroacetamide (1:2 mol:mol),
(ii) 50% Betaine:Trifluoroacetamide (1:2 mol:mol)/50% PEG 200,
(iii) 10% Betaine:Trifluoroacetamide (1:2 mol:mol)/90% PEG 200.

Cycle parameters were 94° C.×8 sec, 55° C.×8 sec and 72° C.×10 sec for 30 cycles. PCR products were visualised following gel electrophoresis and staining with ethidium bromide. A large amount of PCR product resulted from this amplification, equal or greater to the amount generated by an identical procedure using BMV RNA as a template. Results are shown in FIG.

This demonstrates that samples treated with dilutions of mixtures of a quaternary ammonium compound and a halogenated organic compound can be successfully analysed by RNA analysis techniques such as PCR.

### Example 41. Inactivation of SARS-CoV-2 Using Betaine:Trifluoroacetamide Diluted with Ethanol

A composition comprising 95% by weight of the deep eutectic solvent Betaine:Trifluoroacetamide (1:2 mol:mol) and 5% ethanol was prepared.

A tissue culture fluid containing 5% (v/v) foetal calf serum spiked with the “England 2” strain of SARS-CoV-2 was prepared.

Samples of the composition were mixed with the tissue culture fluid at a ratio of 3:1 by volume. Control samples, comprising phosphate buffered saline (“PBS”) in place of the composition, were also prepared.

The samples were then incubated at room temperature for 10 minutes or for 30 minutes. All samples were subsequently subjected to a purification step to remove cytotoxic buffer components.

Two tests were carried out. All measurements were performed in triplicate

Test 1. Purified samples were immediately titrated on Vero E6 cells to establish virus titre. This test is quantitative and reports the titre of virus in each treatment condition in TCID50 per ml. Reduction in virus titre following treatment is given as the difference between the mean log₁₀ TCID50/ml for treated conditions and the PBS control.

Test 2. In parallel, purified samples were seeded onto Vero E6 monolayers to amplify any remaining virus over the course of up to four serial passages. Virus amplification over each passage was detected by visual (microscopic) examination of monolayers for cytopathic effect, and confirmed by SARS-CoV-2-specific real-time PCR. This test is qualitative and reports either the presence or absence of virus amplification. This test may detect levels of virus that are below the detection limit of the titration assay (test 1) due to a greater sample plating volume and the opportunity for any virus present to amplify over serial passages.

The results of the tests are shown in the tables below.

TABLE 41.1
Results after 10 minute incubation

| | Maximum detectable virus reduction in | | |
|---|---|---|---|
| | test 1 (log₁₀ TCID 50/ml) | | 6.4 |
| | Test 1: Virus titration post-treatment | | Test 2: passage of samples in |
| | Mean virus titre | Titre reduction | cell culture |
| Sample | (log₁₀ TCID50/ml) | (log₁₀ TCID50/ml) | Detected/not detected |
| PBS-treated | 7.4 | — | Virus detected (all replicates) |
| (control) | | | |
| Test buffer- | ≤1.0 | ≥6.4 | Virus not detected |
| treated (10 | | | |
| minutes) | | | |

TABLE 41.2
Results after 30 minute incubation

| | Maximum detectable virus reduction in | | |
|---|---|---|---|
| | test 1 (log₁₀ TCID 50/ml) | | 6.4 |
| | Test 1: Virus titration post-treatment | | Test 2: passage of samples in |
| | Mean virus titre | Titre reduction | cell culture |
| Sample | (log₁₀ TCID50/ml) | (log₁₀ TCID50/ml) | Detected/not detected |
| PBS-treated | 8.1 | — | Virus detected (all replicates) |
| (control) | | | |
| Test buffer- | ≤1.0 | ≥7.1 | Virus not detected |
| treated (10 | | | |
| minutes) | | | |

In test 1, the composition reduced virus titre to below the limit of detection for the tests (corresponding to 6.4 and 7.1 log 10 reductions at 10 and 30 minutes respectively.

In test 2, no virus was detectable from treated replicates for either treatment time, after four serial passages in cell culture.

These results are consistent with complete inactivation of the virus by the composition.

In addition, the ethanol dilution of Betaine:Trifluoroacetamide was found to be less viscous than undiluted Betaine:Trifluoroacetamide, and could be reliably handled by a pipetting robot.

### Example 42. Inactivation of SARS-CoV-2 Using Betaine:Trifluoroacetamide Diluted with Ethanol and Water

A composition comprising 85% by weight of the deep eutectic solvent Betaine:Trifluoroacetamide (1:2 mol:mol), 10% water by weight, and 5% ethanol by weight was prepared. The composition was observed to have even lower viscosity than the composition of Example 41.

A tissue culture fluid containing 5% (v/v) foetal calf serum spiked with the England 2 strain of SARS-CoV-2 was prepared and concentrated through a 100 Kda molecular weight cut-off centrifugal filter.

Triplicate samples of the tissue culture fluid were treated with the composition for and incubated for 10 or 30 minutes, or mock-treated in triplicate with an equivalent volume of PBS. All samples were then diluted two-fold to reduce product viscosity and subjected to a purification step to remove cytotoxic buffer components. PBS-treated samples were subjected to the same dilution and purification procedure in parallel.

Purified samples were titrated on Vero E6 cells to establish virus titre. This test is quantitative and reports the titre of virus in each treatment condition in TCID50 per ml. Reduction in virus titre following treatment is given as the difference between the mean log 10 TCID50/ml for treated conditions and the PBS control. The results are shown in the table below.

TABLE 42.1

| | Mean virus titre in log10 | Titre reduction in log10 |
|---|---|---|
| | TCID50/ml [95% confidence | TCID50/ml [95% confidence |
| | interval] | interval] |
| PBS-treated control | 7.0 [6.7-7.3] | |
| Example composition (10 | ≤1.8^{†} | ≥5.1 [4.8-5.5] |
| minute incubation time) | | |
| Example composition (30 | ≤1.0* | ≥6.0 [5.7-6.3] |
| minute incubation time) | | |

^{†}Limit of detection for 10 minute test was 1.8 log10 TCID50/ml
*Limit of detection for 30 minute test was 1.0 log10 TCID50/ml

Treatment with the example composition for 10 or 30 minutes reduced virus titre to below the limit of detection of the test. This represented a titre reduction of 5.1 log 10 TCID50/ml for the 10 minute treatment condition and 6.0 log 10 TCID50/ml for the 30 minute treatment condition.

This demonstrates that dilutions of mixtures of a quaternary ammonium compound and a halogenated organic compound with ethanol and water are effective for inactivating viruses such as SARS-CoV-2.

### Example 43. Inactivation of SARS-CoV-2 Using Betaine:Trifluoroacetamide Diluted with PEG 200

A composition comprising 10% by weight of the deep eutectic solvent Betaine:Trifluoroacetamide (1:2 mol:mol) and 90% by weight PEG 200 was prepared.

A tissue culture fluid containing 5% (v/v) foetal calf serum spiked with the “England 2” strain of SARS-CoV-2 was prepared.

Triplicate samples of the tissue culture fluid were treated with the composition 10 minutes or 30 minutes, or mock-treated in triplicate with an equivalent volume of PBS. All samples were then diluted two-fold to reduce product viscosity and subjected to a purification step to remove cytotoxic buffer components. PBS-treated samples were subjected to the same dilution and purification procedure in parallel.

Purified samples were titrated on Vero E6 cells to establish virus titre. This test is quantitative and reports the titre of virus in each treatment condition in TCID50 per ml. Reduction in virus titre following treatment is given as the difference between the mean log₁₀ TCID50/ml for treated conditions and the PBS control. The results are shown in the table below.

| | Mean virus titre in log10 | Titre reduction in log10 |
|---|---|---|
| | TCID50/ml [95% confidence | TCID50/ml [95% confidence |
| | interval] | interval] |
| PBS-treated | 5.8 [5.5-6.1] | — |
| Test buffer-treated | ≤1.4 ^{†} * | ≥4.4 [4.1-4.6] |
| (10 minutes) | | |
| Test buffer-treated | ≤1.0^{†} * | ≥4.8 [4.5-5.1] |
| (30 minutes) | | |

Treatment with the test composition reduced SARS-CoV-2 titre by 4.4 and 4.8 log 10 TCID50/ml after 10 and 30 minutes, respectively. 30 minute treatment reduced levels of virus to below the limit of detection of the test.

This demonstrates that even a dilute solution of a mixture of a quaternary ammonium compound with a halogenated organic compound is effective for inactivating viruses.

### Example 44. Inactivation of Duck Hepatitis B Virus Using Betaine:Trifluoroacetamide

A 200 μL aliquot of test virus suspension comprising Duck Hepatitis B virus, Strain 911115 in whole duck serum was added to 1800 μL of the test substance and mixed using a vortex mixer.

The solution (10⁻¹ dilution) was held for the remainder of the 60 minute exposure time at 20±2° C. immediately following the exposure time, a 200 μL aliquot of the solution was transferred to a tube containing 1.8 mL of fetal bovine serum (10₋₂ dilution). To reduce the cytotoxic effect of the test substance to indicator cell cultures, the 10⁻² dilution was passed through a Sephadex LH-20 gel column prior to performing subsequent serial dilutions in test medium. The indicator cell cultures used were primary duck hepatocytes.

A virus control, cytotoxicity control, and neutralization control were assayed in parallel and included the use of a Sephadex LH-20 gel column for each parameter, as done in the test. Antiviral properties of the composition were evaluated and compared at the specified time intervals.

Results are shown in the tables below.

| | | Test: |
|---|---|---|
| | | Duck Hepatitis B Virus + |
| Dilution | Virus Control | RNAssist |
| Cell Control | 0 0 0 0 | 0 0 0 0 |
| 10⁻² | + + + + | 0 0 0 0 |
| 10⁻³ | + + + + | 0 0 0 0 |
| 10⁻⁴ | + + + + | 0 0 0 0 |
| 10⁻⁵ | + + + + | 0 0 0 0 |
| 10⁻⁶ | 0 0 0 0 | 0 0 0 0 |
| 10⁻⁷ | 0 0 0 0 | 0 0 0 0 |
| TCID₅₀/250 μL | 10^{5.50} | ≤10^{1.50} |
| Percent Reduction | NA | ≥99.9% |
| Log Reduction | NA | ≥4.00 log₁₀ |

| | | Neutralization Control |
|---|---|---|
| | Cytotoxicity Control | Duck Hepatitis B virus + |
| Dilution | RNAssist | RNAssist |
| Cell Control | 0 0 0 0 | 0 0 0 0 |
| 10⁻² | 0 0 0 0 | + + + + |
| 10⁻³ | 0 0 0 0 | + + + + |
| 10⁻⁴ | 0 0 0 0 | + + + + |
| TCID₅₀/250 μL | ≤10^{1.50} | Neutralized at a TCID₅₀/250 |
| | | μL of ≤1.50 Log₁₀ |

The results demonstrate that Betaine:Trifluoroacetamide is effective for inactivating Duck Hepatitis B, which is a surrogate for human hepatitis B and for HIV.

### Example 44. Inactivation of Further Viruses Using Betaine:Trifluoroacetamide

Further viral inactivation tests were performed using Vaccinia Elstree Strain, Bovine Viral Diarrhoea Virus VR-1422, and Feline immunodeficiency virus VR-1312. 0.9 mL of Betaine:Trifluoroacetamide was added to 0.1 mL of inocula comprising each of these viruses. A reduction in activity of greaterthan 4 log units was observed in less than 60 minutes for all of the samples investigated.

This demonstrates that Betaine:Trifluoroacetamide is effective for inactivating viruses.

### Example 45. Viscosity Reduction by Dilution

The compositions desirably have as low a viscosity as possible. This may allow the composition to be manipulated more easily, e.g. by pipetting, or by a liquid handling robot. Adding a solvent may reduce viscosity.

Viscosities of some selected pure glycols as measured by the falling ball bearing method at 20° C. are as follows:
Polyethylene glycol 200: 64 cP,
Polyethylene glycol 300: 89 cP,
Tetraethylene glycol: 44 cP,
Polyethylene glycol 200 Dimethacrylate: 17 cP
Tetraethylene glycol monomethyl ether: 12 cP
Tetraethylene glycol dimethyl ether: 3.3 cP.

The viscosity (in centipoises) of the Betaine:Trifluoroacetamide compositions and diluted mixtures are:
Betaine:Trifluoroacetamide (1:2 mol:mol) (320 cP),
10% Betaine:Trifluoroacetamide (1:2 mol:mol) 90% PEG 200 (96 cP)
50% Betaine:Trifluoroacetamide (1:2 mol:mol) 50% PEG 200 (144 cP)
10% Betaine:Trifluoroacetamide (1:2 mol:mol) 90% Tetraethylene glycol (66 cP)
10% Betaine:Trifluoroacetamide (1:2 mol:mol) 90% Tetraethylene glycol monomethyl ether (24 cP)
10% Betaine:Trifluoroacetamide (1:2 mol:mol) 90% Tetraethylene glycol dimethyl ether (9 cP)
95% Betaine:Trifluoroacetamide (1:2 mol mol) 5% Ethanol (224 cP)
85% Betaine:Trifluoroacetamide (1:2 mol mol) 5% Ethanol 10% water (44 cP)
10% Betaine:Trifluoroacetamide (1:2 mol:mol) 90% water (6 cP) at 20° C.

It was found that the addition of 22% final volume PEG 200 to Betaine:Trifluoroacetamide reduced by 50%.

These results demonstrate that significant reductions in viscosity are possible by diluting the Betaine:Trifluoroacetamide.

Other variants or use cases of the disclosed techniques may become apparent to the person skilled in the art once given the disclosure herein. The scope of the disclosure is not limited by the described embodiments but only by the accompanying claims.

1. A method of inactivating a virus, comprising contacting the virus with a composition comprising:
a quaternary ammonium compound; and
a halogenated organic compound;
wherein the quaternary ammonium compound is a compound of Formula 1 or a salt thereof: 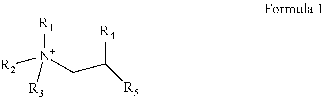
wherein:
R1, R2 and R3 are each independently selected from a methyl group and ethyl group;
R4 is H or OH;
R5 is selected from H, a halide, a carbonyl oxygen, a methyl group, and 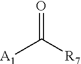
wherein:
A1 is selected from CH₂, O and S;
R7 is selected from OH, an unsubstituted C1 to C3 alkyl group, an alkyl carboxylic acid group having 1 to 3 carbon atoms, and a substituted C1 to C3 alkyl group bearing one or more substituents selected from hydroxyl and halide groups;
with the proviso that, when R5 is H or a methyl group, R4 is OH;
and wherein the halogenated organic compound is a compound of Formula 2: 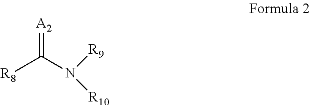
wherein:
A2 is O or S;
R8 is a substituted C1 to C12 alkyl group bearing one or more substituents selected from fluoro, chloro, bromo, and iodo; and
i) R9 and R10 are each independently selected from H, an unsubstituted C1 to C12 alkyl group; an unsubstituted alkyl ester group having 2 to 12 carbon atoms; an alkyl ester group having 2 to 12 carbon atoms and bearing one or more substituents selected from fluoro, chloro, bromo, iodo, and hydroxyl; and a substituted C1 to C12 alkyl group bearing one or more substituents selected from fluoro, chloro, bromo, iodo, and hydroxyl; or
ii) R9 and R10 together form an imidazole group.

2. The method according to claim 1, wherein the composition further comprises a solvent having a structure of Formula 3: 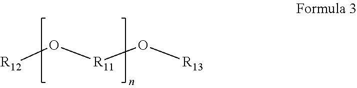
wherein:
R11 is a methylene group or a C2 to C6 linear or branched alkylene group;
R12 and R13 are each independently selected from H, a C1 to C6 alkyl group, an acrylate group, a methacrylate group, and an oxolan-2-ylmethylene group; and
wherein n is in the range 1 to 14,
with the proviso that when n is 1, R11 is not methylene or at least one of R12 and R13 is not H.

3-9. (canceled)

10. The method according to claim 2, wherein the solvent has a molecular weight in the range 50 to 600 Da and is selected from the group consisting of tripropylene glycol, tripropylene glycol monomethyl ether, tripropylene glycol dimethyl ether, tetrapropylene glycol, tetrapropylene glycol monomethyl ether, tetrapropylene glycol dimethyl ether, tetraethylene glycol, and tetraethylene glycol monomethyl ether.

11-14. (canceled)

15. The method according to claim 1, wherein the composition further comprises a solvent selected from water, C1 to C6 alkanol, a polypropylene glycol-polyethylene glycol co-polymer, a polyethylene glycol alkyl ether, glycerol, ethylene glycol, diethylene glycol, triethylene glycol, tetraethylene glycol, and/or a polyethylene glycol.

16. (canceled)

17. The method according to claim 2, wherein the solvent is present in an amount of 50 to 98%, by volume based on the volume of the composition.

18. The method according to claim 1, wherein the quaternary ammonium compound is present in the composition at a concentration in the range 0.1 mM to 400 mM.

19. The method according to claim 1, wherein the halogenated organic compound is present in the composition at a concentration in the range 0.1 to 850 mM.

20. The method according to claim 1, wherein the virus is present in a biological sample comprising mucus, sputum, saliva, and/or breath condensate.

21. The method according to claim 1, further comprising, after inactivating the virus, analysing the biological sample to detect or identify the virus.

22. The method according to claim 21, wherein no purification is performed between the inactivation and the analysis.

23. (canceled)

24. The method according to claim 1, wherein the molar ratio of the quaternary ammonium compound to the halogenated organic compound is in the range 1:3 to 2:1.

25. (canceled)

26. The method according to claim 1, wherein the quaternary ammonium compound is selected from N,N,N-trimethylglycine, choline and a halocholine.

27-32. (canceled)

33. The method according to claim 1, wherein the halogenated organic compound is selected from: fluoroacetamide, difluoroacetamide, trifluoroacetamide, trifluorothioacetamide, chloroacetamide, dichloroacetamide, trichloroacetamide, chlorofluoroacetamide, chlorodifluoroacetamide, dichlorofluoroacetamide, N-methyl-fluoroacetamide, N-methyl-difluoroacetamide, N-methyl-trifluoroacetamide, N-methyl-chloroacetamide, N-methyl-dichloroacetamide, N-methyl-trichloroacetamide, N-methyl-chlorofluoroacetamide, N-methyl-chlorodifluoroacetamide, and N-methyl-dichlorofluoroacetamide.

34. (canceled)

35. The method according to claim 1, wherein the quaternary ammonium compound is N,N,N-trimethyl glycine, and the halogenated organic compound is trifluoroacetamide.

36-55. (canceled)

56. A method for detecting an analyte in a sample, wherein the analyte is a biomolecule selected from an RNA and a DNA, the method comprising:
preparing the sample for nucleic acid analysis by contacting the sample with a deep eutectic solvent; and
performing nucleic acid analysis to detect the analyte,
wherein the deep eutectic solvent in activates a virus in the sample, wherein the nucleic acid analysis is carried out in the presence of crude cellular components and wherein the deep eutectic solvent is trimethylglycine:trifluoroacetamide.

57-68. (canceled)

69. The method according to claim 56, further comprising concentrating a virus in the sample by contacting the sample with a membrane, bead, or particle having a surface configured to bind to the virus, wherein the virus is SARS-CoV-2, and wherein the surface comprises an ACE2 protein.

70-83. (canceled)

84. The method according to claim 56, wherein the deep eutectic solvent is present in a mixture further comprising a solvent according to Formula 3: 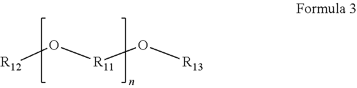
wherein:
R11 is a methylene group or a C2 to C6 linear or branched alkylene group;
R12 and R13 are each independently selected from H, a C1 to C6 alkyl group, an acrylate group, a methacrylate group, and an oxolan-2-ylmethylene group; and
wherein n is in the range 1 to 14,
with the proviso that when n is 1, R11 is not methylene or at least one of R12 and R13 is not H.

85-93. (canceled)

94. The method according to claim 56, wherein the deep eutectic solvent is present in a mixture further comprising a solvent selected from water, a C1 to C6 alkanol, or mixtures thereof.

95-96. (canceled)

97. A method of manufacturing a vaccine for preventing a viral disease, which method comprises:
preparing an inactivated virus by contacting a virus with a composition comprising a quaternary ammonium compound and a halogenated organic compound; and
formulating the inactivated virus in a pharmaceutically-acceptable carrier, wherein the quaternary ammonium compound is a compound of Formula 1 or a salt thereof: 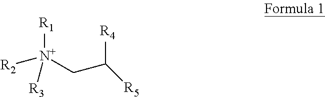
wherein:
R1, R2 and R3 are each independently selected from a methyl group and ethyl group:
R4 is H or OH:
R5 is selected from H, a halide, a carbonyl oxygen, a methyl group, and 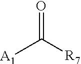
wherein:
A1 is selected from CH₂, O and S;
R7 is selected from OH, an unsubstituted C1 to C3 alkyl group, an alkyl carboxylic acid group having 1 to 3 carbon atoms, and a substituted C1 to C3 alkyl group bearing one or more substituents selected from hydroxyl and halide groups;
with the proviso that, when R5 is H or a methyl group, R4 is OH;
and wherein the halogenated organic compound is a compound of Formula 2: 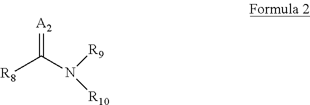
wherein:
A2 is O or S;
R8 is a substituted C1 to C12 alkyl group bearing one or more substituents selected from fluoro, chloro, bromo, and iodo; and
i) R9 and R10 are each independently selected from H, an unsubstituted C1 to C12 alkyl group: an unsubstituted alkyl ester group having 2 to 12 carbon atoms; an alkyl ester group having 2 to 12 carbon atoms and bearing one or more substituents selected from fluoro, chloro, bromo, iodo, and hydroxyl; and a substituted C1 to C12 alkyl group bearing one or more substituents selected from fluoro, chloro, bromo, iodo, and hydroxyl; or
ii) R9 and R10 together form an imidazole group.

98-99. (canceled)

100. The method according to claim 97, wherein the virus is SARS-CoV-2.

101-102. (canceled)


SEQUENCE LISTING (5 sequences)

SEQ ID NO: 1 - DNA, 22 bp, Artificial Sequence
  SARS-CoV-2 reverse transcriptase primer
atattgcagcagtacgcacaca

SEQ ID NO: 2 - DNA, 26 bp, Artificial Sequence
  SARS-CoV-2 target-specific forward primer
acaggtacgttaatagttaatagcgt

SEQ ID NO: 3 - DNA, 26 bp, Artificial Sequence
  real-time labelled probe
acactagccatccttactgcgcttcg

SEQ ID NO: 4 - DNA, 20 bp, Artificial Sequence
  BMV F Primer
ctatcaccaagatgtcttcg

SEQ ID NO: 5 - DNA, 20 bp, Artificial Sequence
  BMV R primer
gagccccagcgcactcggtc